SEQUENCE LISTING (179 sequences)

SEQ ID NO: 1 - protein, 144 aa, Artificial Sequence
  Dimerization unit
ELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 2 - protein, 19 aa, Artificial Sequence
  Signal peptide of mIg VH
MNFGLRLIFLVLTLKGVQC

SEQ ID NO: 3 - protein, 650 aa, Artificial Sequence
  VB5049*
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEAQPGSGEGRGSLLTCGDVEENPGPMPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 4 - protein, 280 aa, Artificial Sequence
  VB5052*
MQVSTAALAVLLCTMALCNQVLSAPLAADTPTACCFSYTSRQIPQNFIADYFETSSQCSKPSVIFLTKRGRQVCADPSEEWAVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEAQP

SEQ ID NO: 5 - protein, 56 aa, Artificial Sequence
  VB5051*
MNFGLRLIFLVLTLKGVQCSPGKNATGMEVGWYRSPFSRVVHLYRNGKDQDAEAQP

SEQ ID NO: 6 - protein, 18 aa, Artificial Sequence
  Self-cleaving peptide
EGRGSLLTCGDVEENPGP

SEQ ID NO: 7 - protein, 19 aa, Artificial Sequence
  Self-cleaving peptide
ATNFSLLKQAGDVEENPGP

SEQ ID NO: 8 - protein, 20 aa, Artificial Sequence
  Self-cleaving peptide
QCTNYALLKLAGDVESNPGP

SEQ ID NO: 9 - protein, 22 aa, Artificial Sequence
  Self-cleaving peptide
VKQTLNFDLLKLAGDVESNPGP

SEQ ID NO: 10 - DNA, 81 bp, Artificial Sequence
  Nucleotide sequence encoding amino acids 1-27 of SEQ ID NO 1
gagctcaaaaccccacttggtgacacaactcacacagagcccaaatcttgtgacacacctcccccgtgcccaaggtgccca

SEQ ID NO: 11 - DNA, 321 bp, Artificial Sequence
  Nucleotide sequence encoding amino acids 38-144 of SEQ ID NO 1
ggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 12 - DNA, 432 bp, Artificial Sequence
  Nucleotide sequence encoding the amino acid sequence of SEQ ID NO 1
gagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 13 - protein, 23 aa, Artificial Sequence
  Signal peptide hCCL3L1
MQVSTAALAVLLCTMALCNQVLS

SEQ ID NO: 14 - protein, 70 aa, Artificial Sequence
  hCCL3L1
APLAADTPTACCFSYTSRQIPQNFIADYFETSSQCSKPSVIFLTKRGRQVCADPSEEWVQKYVSDLELSA

SEQ ID NO: 15 - protein, 246 aa, Artificial Sequence
  scFv with specificity for murine CD205
DIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSS

SEQ ID NO: 16 - protein, 37 aa, Artificial Sequence
  MOG (27-63)*
SPGKNATGMEVGWYRSPFSRVVHLYRNGKDQDAEAQP

SEQ ID NO: 17 - protein, 18 aa, Artificial Sequence
  Signal peptide mIL-10
MPGSALLCCLLLLTGMRI

SEQ ID NO: 18 - protein, 160 aa, Artificial Sequence
  mIL-10
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 19 - protein, 37 aa, Artificial Sequence
  MOG (27-63)
SPGKNATGMEVGWYRSPFSRVVHLYRNGKDQDAEQAP

SEQ ID NO: 20 - protein, 862 aa, Artificial Sequence
  VB5062
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAPGSGEGRGSLLTCGDVEENPGPMPPSGLRLLPLLLPLPWLLVLTPGRPAAGLSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 21 - protein, 633 aa, Artificial Sequence
  VB5063
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAPGSGEGRGSLLTCGDVEENPGPMACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSD

SEQ ID NO: 22 - protein, 583 aa, Artificial Sequence
  VB5064
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 23 - protein, 569 aa, Artificial Sequence
  VB5065
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMNATHCILALQLFLMAVSGCYCHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 24 - protein, 29 aa, Artificial Sequence
  Signal peptide mTGF beta 1
MPPSGLRLLPLLLPLPWLLVLTPGRPAAG

SEQ ID NO: 25 - protein, 361 aa, Artificial Sequence
  mTGF beta 1
LSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 26 - protein, 35 aa, Artificial Sequence
  Signal peptide mCLTA-4
MACLGLRRYKAQLQLPSRTWPFVALLTLLFIPVFS

SEQ ID NO: 27 - protein, 126 aa, Artificial Sequence
  mCLTA-4 extracellular domain
  DOMAIN (1)..(126): extracellular domain
EAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSD

SEQ ID NO: 28 - protein, 20 aa, Artificial Sequence
  Signal peptide mIL-2
MYSMQLASCVTLTLVLLVNS

SEQ ID NO: 29 - protein, 149 aa, Artificial Sequence
  mIL-2
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 30 - protein, 22 aa, Artificial Sequence
  Signal peptide mIFN gamma
MNATHCILALQLFLMAVSGCYC

SEQ ID NO: 31 - protein, 133 aa, Artificial Sequence
  mIFN gamma
HGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 32 - protein, 650 aa, Artificial Sequence
  VB5049
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAPGSGEGRGSLLTCGDVEENPGPMPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 33 - protein, 280 aa, Artificial Sequence
  VB5052
MQVSTAALAVLLCTMALCNQVLSAPLAADTPTACCFSYTSRQIPQNFIADYFETSSQCSKPSVIFLTKRGRQVCADPSEEWAVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAP

SEQ ID NO: 34 - protein, 56 aa, Artificial Sequence
  VB5051
MNFGLRLIFLVLTLKGVQCSPGKNATGMEVGWYRSPFSRVVHLYRNGKDQDAEQAP

SEQ ID NO: 35 - protein, 451 aa, Artificial Sequence
  VB5048
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAP

SEQ ID NO: 36 - protein, 1062 aa, Artificial Sequence
  VB5044
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAPGSGEGRGSLLTCGDVEENPGPMPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSGSGATNFSLLKQAGDVEENPGPMPPSGLRLLPLLLPLPWLLVLTPGRPAAGLSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 37 - protein, 1224 aa, Artificial Sequence
  VB5054
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAPGSGEGRGSLLTCGDVEENPGPMPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKSGSGATNFSLLKQAGDVEENPGPMPPSGLRLLPLLLPLPWLLVLTPGRPAAGLSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCSGSGEGRGSLLTCGDVEENPGPMWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 38 - protein, 17 aa, Artificial Sequence
  Signal peptide mGM-CSF
MWLQNLLFLGIVVYSLS

SEQ ID NO: 39 - protein, 124 aa, Artificial Sequence
  mGM-CSF
APTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 40 - protein, 476 aa, Artificial Sequence
  VB5068
MKLVSIFLLVTIGICGYSATALLINRLPVVDKLPVPLDDIIPSFDPLKMLLKTLGISVEHLVTGLKKCVDELGPEASEAVKKLLEALSHLVELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAPGSGEGRGSLLTCGDVEENPGPMPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 41 - protein, 625 aa, Artificial Sequence
  VB5069
MTRRRSAPASWLLVSLLGVATSLEVSESPGSVQVARGQTAVLPCAFSTSAALLNLNVIWMVIPLSNANQPEQVILYQGGQMFDGALRFHGRVGFTGTMPATNVSIFINNTQLSDTGTYQCLVNNLPDRGGRNIGVTGLTVLVPPSAPQCQIQGSQDLGSDVILLCSSEEGIPRPTYLWEKLDNTLKLPPTATQDQVQGTVTIRNISALSSGLYQCVASNAIGTSTCLLDLQVISPQPRSVELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAPGSGEGRGSLLTCGDVEENPGPMPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 42 - protein, 554 aa, Artificial Sequence
  VB5070
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLSPGKNATGMEVGWYRSPFSRVVHLYRNGKDQDAEQAPGSGEGRGSLLTCGDVEENPGPMPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 43 - protein, 21 aa, Artificial Sequence
  Signal peptide mSCGB3A2
MKLVSIFLLVTIGICGYSATA

SEQ ID NO: 44 - protein, 70 aa, Artificial Sequence
  mSCGB3A2
LLINRLPVVDKLPVPLDDIIPSFDPLKMLLKTLGISVEHLVTGLKKCVDELGPEASEAVKKLLEALSHLV

SEQ ID NO: 45 - protein, 22 aa, Artificial Sequence
  Signal peptide mVSIG3
MTRRRSAPASWLLVSLLGVATS

SEQ ID NO: 46 - protein, 218 aa, Artificial Sequence
  mVSIG3 extracellular domain
  DOMAIN (1)..(218): extracellular domain
LEVSESPGSVQVARGQTAVLPCAFSTSAALLNLNVIWMVIPLSNANQPEQVILYQGGQMFDGALRFHGRVGFTGTMPATNVSIFINNTQLSDTGTYQCLVNNLPDRGGRNIGVTGLTVLVPPSAPQCQIQGSQDLGSDVILLCSSEEGIPRPTYLWEKLDNTLKLPPTATQDQVQGTVTIRNISALSSGLYQCVASNAIGTSTCLLDLQVISPQPRSV

SEQ ID NO: 47 - protein, 24 aa, Artificial Sequence
  Signal peptide mPD-1
MWVRQVPWSFTWAVLQLSWQSGWL

SEQ ID NO: 48 - protein, 145 aa, Artificial Sequence
  mPD-1 extracellular domain
  DOMAIN (1)..(145): extracellular domain
LEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGM

SEQ ID NO: 49 - protein, 784 aa, Artificial Sequence
  VB5076
MNFGLRLIFLVLTLKGVQCDIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSSELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLKEVDRLEDELVNEKEKYKSIGGGGSGGGGSAYKEQIKTLTNKLKAAEARAEGGGGSGGGGSNQLKEARFLAEEADRKYDEVGGGGSGGGGSAALNRRIQLLEEDLERSEERGGGGSGGGGSDLDQVQESLLKANNQLVEKDGGGGSGGGGSEQQNKEANNRAEKSEEEVHNGSGEGRGSLLTCGDVEENPGPMPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 50 - protein, 171 aa, Artificial Sequence
  Met e 1
KEVDRLEDELVNEKEKYKSIGGGGSGGGGSAYKEQIKTLTNKLKAAEARAEGGGGSGGGGSNQLKEARFLAEEADRKYDEVGGGGSGGGGSAALNRRIQLLEEDLERSEERGGGGSGGGGSDLDQVQESLLKANNQLVEKDGGGGSGGGGSEQQNKEANNRAEKSEEEVHN

SEQ ID NO: 51 - protein, 161 aa, Artificial Sequence
  hCTLA4 extracellular domain
  SIGNAL (1)..(35): Signal peptide
  DOMAIN (36)..(161): extracellular domain
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 52 - protein, 170 aa, Artificial Sequence
  hPD-1 extracellular domain
  SIGNAL (1)..(23): signal peptide
  DOMAIN (24)..(170): extracellullar domain
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 53 - protein, 178 aa, Artificial Sequence
  hIL-10
  SIGNAL (1)..(18): signal peptide
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 54 - protein, 390 aa, Artificial Sequence
  hTGF beta - 1
  SIGNAL (1)..(29): signal peptide
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 55 - protein, 153 aa, Artificial Sequence
  hIL-2
  SIGNAL (1)..(20): signal peptide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 56 - protein, 144 aa, Artificial Sequence
  hGM-CSF
  SIGNAL (1)..(17): signal peptide
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 57 - protein, 166 aa, Artificial Sequence
  hIFN- gamma
  SIGNAL (1)..(23): signal peptide
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 58 - protein, 442 aa, Artificial Sequence
  hTGF beta 2
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 59 - protein, 412 aa, Artificial Sequence
  hTGF beta 3
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 60 - DNA, 1173 bp, Artificial Sequence
  hTGF beta 1
atgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctga

SEQ ID NO: 61 - DNA, 1329 bp, Artificial Sequence
  hTGF beta 2
atgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaa

SEQ ID NO: 62 - DNA, 930 bp, Artificial Sequence
  hTGF beta 3
atgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccggtga

SEQ ID NO: 63 - DNA, 537 bp, Artificial Sequence
  hIL-10
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 64 - protein, 93 aa, Artificial Sequence
  hSCGB3A2
  SIGNAL (1)..(21): signal peptide
MKLVTIFLLVTISLCSYSATAFLINKVPLPVDKLAPLPLDNILPFMDPLKLLLKTLGISVEHLVEGLRKCVNELGPEASEAVKKLLEALSHLV

SEQ ID NO: 65 - DNA, 282 bp, Artificial Sequence
  hSCGB3A2
atgaagctggtaactatcttcctgctggtgaccatcagcctttgtagttactctgctactgccttcctcatcaacaaagtgccccttcctgttgacaagttggcacctttacctctggacaacattcttccctttatggatccattaaagcttcttctgaaaactctgggcatttctgttgagcaccttgtggaggggctaaggaagtgtgtaaatgagctgggaccagaggcttctgaagctgtgaagaaactgctggaggcgctatcacacttggtgtga

SEQ ID NO: 66 - protein, 241 aa, Artificial Sequence
  hVSIG-3
  SIGNAL (1)..(22): signal peptide
  DOMAIN (23)..(241): extracellular domain
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIG

SEQ ID NO: 67 - DNA, 723 bp, Artificial Sequence
  hVSIG-3 extracellular domain
  misc_feature (1)..(723): extracellular domain
atgacttctcagcgttcccctctggcgcctttgctgctcctctctctgcacggtgttgcagcatccctggaagtgtcagagagccctgggagtatccaggtggcccggggtcagccagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcaggtttgtaccagtgcgtggcttctaatgctattggaaccagcacctgtcttctggatctccaggttatttcaccccagcccaggaacattgga

SEQ ID NO: 68 - protein, 8 aa, Artificial Sequence
  consensus of 2A self-cleaving peptide; X can be any amino acid
  SITE (2)..(2): X can be any amino acid
  SITE (4)..(4): X can be any amino acid
DXEXNPGP

SEQ ID NO: 69 - protein, 8 aa, Artificial Sequence
  Self-cleaving peptide
DVEENPGP

SEQ ID NO: 70 - protein, 8 aa, Artificial Sequence
  Self-cleaving peptide
DVESNPGP

SEQ ID NO: 71 - protein, 21 aa, Artificial Sequence
  Self-cleaving peptide
GSGEGRGSLLTCGDVEENPGP

SEQ ID NO: 72 - protein, 22 aa, Artificial Sequence
  Self-cleaving peptide
GSGATNFSLLKQAGDVEENPGP

SEQ ID NO: 73 - protein, 23 aa, Artificial Sequence
  Self-cleaving peptide
GSGQCTNYALLKLAGDVESNPGP

SEQ ID NO: 74 - protein, 25 aa, Artificial Sequence
  Self-cleaving peptide
GSGVKQTLNFDLLKLAGDVESNPGP

SEQ ID NO: 75 - protein, 10 aa, Artificial Sequence
  Linker
GGGGSGGGSS

SEQ ID NO: 76 - protein, 5 aa, Artificial Sequence
  Linker
GGGSG

SEQ ID NO: 77 - protein, 5 aa, Artificial Sequence
  Linker
GGSGG

SEQ ID NO: 78 - protein, 6 aa, Artificial Sequence
  Linker
SGSSGS

SEQ ID NO: 79 - protein, 10 aa, Artificial Sequence
  Linker
GGGGSGGGGS

SEQ ID NO: 80 - protein, 5 aa, Artificial Sequence
  Linker
  SITE (1)..(5): can be repeated m times, where m is an integer from 1 to 5
GGGGS

SEQ ID NO: 81 - protein, 5 aa, Artificial Sequence
  Linker
  SITE (1)..(5): can be repeated m times, where m is an integer from 1 to 5
GGGSS

SEQ ID NO: 82 - protein, 5 aa, Artificial Sequence
  Linker
  SITE (1)..(5): can be repeated m times, where m is an integer from 1 to 5
GGSGG

SEQ ID NO: 83 - protein, 5 aa, Artificial Sequence
  Linker
  SITE (1)..(5): can be repeated m times, where m is an integer from 1 to 5
GGGSG

SEQ ID NO: 84 - protein, 6 aa, Artificial Sequence
  Linker
  SITE (1)..(6): can be repeated m times, where m is an integer from 1 to 5
SGSSGS

SEQ ID NO: 85 - protein, 5 aa, Artificial Sequence
  Linker
LGGGS

SEQ ID NO: 86 - protein, 5 aa, Artificial Sequence
  Linker
GLGGS

SEQ ID NO: 87 - protein, 5 aa, Artificial Sequence
  Linker
GGLGS

SEQ ID NO: 88 - protein, 5 aa, Artificial Sequence
  Linker
GGGLS

SEQ ID NO: 89 - protein, 5 aa, Artificial Sequence
  Linker
GGGGL

SEQ ID NO: 90 - protein, 5 aa, Artificial Sequence
  Linker
LGGSG

SEQ ID NO: 91 - protein, 5 aa, Artificial Sequence
  Linker
GLGSG

SEQ ID NO: 92 - protein, 5 aa, Artificial Sequence
  Linker
GGLSG

SEQ ID NO: 93 - protein, 5 aa, Artificial Sequence
  Linker
GGGLG

SEQ ID NO: 94 - protein, 5 aa, Artificial Sequence
  Linker
GGGSL

SEQ ID NO: 95 - protein, 5 aa, Artificial Sequence
  Linker
LGGSS

SEQ ID NO: 96 - protein, 5 aa, Artificial Sequence
  Linker
GLGSS

SEQ ID NO: 97 - protein, 5 aa, Artificial Sequence
  Linker
GGLSS

SEQ ID NO: 98 - protein, 5 aa, Artificial Sequence
  Linker
LGLGS

SEQ ID NO: 99 - protein, 5 aa, Artificial Sequence
  Linker
GLGLS

SEQ ID NO: 100 - protein, 5 aa, Artificial Sequence
  Linker
GLLGS

SEQ ID NO: 101 - protein, 5 aa, Artificial Sequence
  Linker
LGGLS

SEQ ID NO: 102 - protein, 5 aa, Artificial Sequence
  Linker
GLGGL

SEQ ID NO: 103 - protein, 5 aa, Artificial Sequence
  Linker
LGLSG

SEQ ID NO: 104 - protein, 5 aa, Artificial Sequence
  Linker
GLLSG

SEQ ID NO: 105 - protein, 5 aa, Artificial Sequence
  Linker
GGLSL

SEQ ID NO: 106 - protein, 5 aa, Artificial Sequence
  Linker
GGLLG

SEQ ID NO: 107 - protein, 5 aa, Artificial Sequence
  Linker
GLGSL

SEQ ID NO: 108 - protein, 5 aa, Artificial Sequence
  Linker
LGLSS

SEQ ID NO: 109 - protein, 5 aa, Artificial Sequence
  Linker
GGLLS

SEQ ID NO: 110 - protein, 10 aa, Artificial Sequence
  Linker
LGGGSGGGGS

SEQ ID NO: 111 - protein, 10 aa, Artificial Sequence
  Linker
GLGGSGGGGS

SEQ ID NO: 112 - protein, 10 aa, Artificial Sequence
  Linker
GGLGSGGGGS

SEQ ID NO: 113 - protein, 117 aa, Artificial Sequence
  tetramerization unit
AAGCCTCTGGACGGAGAGTATTTCACTCTCCAGATCCGGGGCCCCGAAAGGTTCGAAATGTTCCGGGAGCTTAACGAGGCCTTGGAGCTGAAAGACGCACAGGCCGGAAAGGAACCG

SEQ ID NO: 114 - protein, 10 aa, Artificial Sequence
  Linker
GGGGLGGGGS

SEQ ID NO: 115 - protein, 10 aa, Artificial Sequence
  Linker
LGGSGGGGSG

SEQ ID NO: 116 - DNA, 180 bp, Artificial Sequence
  trimerization unit
gctgggcaggtgaggatctgggccacataccagaccatgctggacaagatccgggaggtgccggagggctggctcatctttgtggccgagagggaagagctctatgtacgcgttagaaatggcttccggaaggtgctgctggaggcccggacagccctcccgagaggcacgggcaatgag

SEQ ID NO: 117 - protein, 27 aa, Artificial Sequence
  trimerization unit
GYIPEAPRDGQAYVRKDGEWVLLSTFL

SEQ ID NO: 118 - protein, 10 aa, Artificial Sequence
  Linker
GGGSSGGGSG

SEQ ID NO: 119 - DNA, 333 bp, Artificial Sequence
  IgM hinge
gccgaactcccgcccaaggtgtccgtgttcgtccctccccgcgatgggttcttcggcaatccacgaaaatccaaactgatttgtcaggccaccggcttctccccccgacagatccaggtgagttggctacgagagggtaaacaggtggggagcggagtgaccactgaccaggtgcaggccgaggccaaggaaagcggacccacaacatacaaagtgacaagcactctgacgattaaggagtcagactggctcggccaatccatgtttacatgccgggttgatcacagagggttgaccttccaacagaacgcatccagtatgtgcgttccagat

SEQ ID NO: 120 - protein, 8 aa, Artificial Sequence
  t cell epitope
SIINFEKL

SEQ ID NO: 121 - protein, 10 aa, Artificial Sequence
  Linker
LGGSSGGGSS

SEQ ID NO: 122 - protein, 10 aa, Artificial Sequence
  Linker
GLGSSGGGSS

SEQ ID NO: 123 - protein, 10 aa, Artificial Sequence
  Linker
GGLSSGGGSS

SEQ ID NO: 124 - protein, 10 aa, Artificial Sequence
  Linker
GGGLSGGGSS

SEQ ID NO: 125 - protein, 10 aa, Artificial Sequence
  Linker
GGGSLGGGSS

SEQ ID NO: 126 - protein, 10 aa, Artificial Sequence
  Linker
LGGGSLGGGS

SEQ ID NO: 127 - protein, 10 aa, Artificial Sequence
  Linker
GLGGSGLGGS

SEQ ID NO: 128 - protein, 10 aa, Artificial Sequence
  Linker
GGLGSGGLGS

SEQ ID NO: 129 - protein, 10 aa, Artificial Sequence
  Linker
GGGLSGGGLS

SEQ ID NO: 130 - protein, 10 aa, Artificial Sequence
  Linker
GGGGLGGGGL

SEQ ID NO: 131 - protein, 10 aa, Artificial Sequence
  Linker
LGGSGLGGSG

SEQ ID NO: 132 - protein, 10 aa, Artificial Sequence
  Linker
GLGSGGLGSG

SEQ ID NO: 133 - protein, 10 aa, Artificial Sequence
  Linker
GGLSGGGLSG

SEQ ID NO: 134 - protein, 10 aa, Artificial Sequence
  Linker
GGGLGGGGLG

SEQ ID NO: 135 - protein, 10 aa, Artificial Sequence
  Linker
GGGSLGGGSL

SEQ ID NO: 136 - protein, 10 aa, Artificial Sequence
  Linker
LGGSSLGGSS

SEQ ID NO: 137 - protein, 10 aa, Artificial Sequence
  Linker
GLGSSGLGSS

SEQ ID NO: 138 - protein, 10 aa, Artificial Sequence
  Linker
GGLSSGGLSS

SEQ ID NO: 139 - protein, 6 aa, Artificial Sequence
  Linker
GSGGGA

SEQ ID NO: 140 - protein, 12 aa, Artificial Sequence
  Linker
GSGGGAGSGGGA

SEQ ID NO: 141 - protein, 18 aa, Artificial Sequence
  Linker
GSGGGAGSGGGAGSGGGA

SEQ ID NO: 142 - protein, 24 aa, Artificial Sequence
  Linker
GSGGGAGSGGGAGSGGGAGSGGGA

SEQ ID NO: 143 - protein, 10 aa, Artificial Sequence
  Linker
GENLYFQSGG

SEQ ID NO: 144 - protein, 10 aa, Artificial Sequence
  Linker
SGGGSSGGGS

SEQ ID NO: 145 - protein, 10 aa, Artificial Sequence
  Linker
SSGGGSSGGG

SEQ ID NO: 146 - protein, 10 aa, Artificial Sequence
  Linker
GGSGGGGSGG

SEQ ID NO: 147 - protein, 10 aa, Artificial Sequence
  Linker
GSGSGSGSGS

SEQ ID NO: 148 - protein, 10 aa, Artificial Sequence
  Linker
GGGSSGGGSG

SEQ ID NO: 149 - protein, 6 aa, Artificial Sequence
  Linker
GGGSSS

SEQ ID NO: 150 - protein, 15 aa, Artificial Sequence
  Linker
GGGSSGGGSSGGGSS

SEQ ID NO: 151 - protein, 8 aa, Artificial Sequence
  Linker
GLGGLAAA

SEQ ID NO: 152 - protein, 12 aa, Artificial Sequence
  Linker
KPEPKPAPAPKP

SEQ ID NO: 153 - protein, 12 aa, Artificial Sequence
  Linker
AEAAAKEAAAKA

SEQ ID NO: 154 - protein, 5 aa, Artificial Sequence
  Linker
  SITE (1)..(5): can be repeated m times, where m is an integer from 1 to 5
EAAAK

SEQ ID NO: 155 - protein, 10 aa, Artificial Sequence
  Linker
GGGLSGGGGS

SEQ ID NO: 156 - protein, 10 aa, Artificial Sequence
  Linker
GLGSGGGGSG

SEQ ID NO: 157 - protein, 10 aa, Artificial Sequence
  Linker
GGLSGGGGSG

SEQ ID NO: 158 - protein, 10 aa, Artificial Sequence
  Linker
GGGLGGGGSG

SEQ ID NO: 159 - protein, 10 aa, Artificial Sequence
  Linker
GGGSLGGGSG

SEQ ID NO: 160 - protein, 15 aa, Artificial Sequence
  Linker
PSRLEEELRRRLTEP

SEQ ID NO: 161 - protein, 8 aa, Artificial Sequence
  Linker
SACYCELS

SEQ ID NO: 162 - protein, 16 aa, Artificial Sequence
  Linker
TQKSLSLSPGKGLGGL

SEQ ID NO: 163 - protein, 13 aa, Artificial Sequence
  Linker
SLSLSPGKGLGGL

SEQ ID NO: 164 - protein, 36 aa, Artificial Sequence
  Linker
GGSAGGSGSGSSGGSSGASGTGTAGGTGSGSGTGSG

SEQ ID NO: 165 - protein, 36 aa, Artificial Sequence
  Linker
GGSGGGSEGGGSEGGGSEGGGSEGGGSEGGGSGGGS

SEQ ID NO: 166 - protein, 15 aa, Artificial Sequence
  Linker
EPKSCDTPPPCPRCP

SEQ ID NO: 167 - protein, 13 aa, Artificial Sequence
  T cell epitope
HCLGKWLGHPDKF

SEQ ID NO: 168 - protein, 29 aa, Artificial Sequence
  T cell epitope
AHSLERVCHCLGKWLGHPDKFVGITYALT

SEQ ID NO: 169 - protein, 14 aa, Artificial Sequence
  T cell epitope
NTWTTCQSIAFPSK

SEQ ID NO: 170 - protein, 30 aa, Artificial Sequence
  T cell epitope
AVPVYIYFNTWTTCQSIAFPSKTSASIGSL

SEQ ID NO: 171 - protein, 21 aa, Artificial Sequence
  T cell epitope
VHFFKNIVTPRTPPPSQGKGR

SEQ ID NO: 172 - protein, 36 aa, Artificial Sequence
  T cell epitope
RTQDENPVVHFFKNIVTPRTPPPSQGKGRGLSLSRF

SEQ ID NO: 173 - protein, 12 aa, Artificial Sequence
  Linker
HEYGAEALERAG

SEQ ID NO: 174 - protein, 5 aa, Artificial Sequence
  Linker
GLSGL

SEQ ID NO: 175 - protein, 5 aa, Artificial Sequence
  Linker
GGGGS

SEQ ID NO: 176 - protein, 5 aa, Artificial Sequence
  Linker
EAAAK

SEQ ID NO: 177 - protein, 7 aa, Artificial Sequence
  Linker
  SITE (1)..(5): can be repeated m times, where m is an integer from 1 to 5
EAAAKGS

SEQ ID NO: 178 - protein, 6 aa, Artificial Sequence
  Linker
  SITE (1)..(4): can be repeated m times, where m is an integer from 1 to 5
EAAKGS

SEQ ID NO: 179 - protein, 17 aa, Artificial Sequence
  Linker
GPSRLEEELRRRLTEPG

(19) United States
(12) Patent Application Publication
(10) Pub. No.: US 2024/0252625 A1
(43) Pub. Date: Aug. 1, 2024

(54) CO-EXPRESSION OF CONSTRUCTS AND IMMUNOINHIBITORY COMPOUNDS

(71) Applicant: Nykode Therapeutics ASA, Oslo (NO)

(72) Inventors: Agnete Brunsvik Fredriksen, Rælingen (NO); Audun Trygge Haugen Bersaas, Oslo (NO); Stine Granum, Oslo (NO); Heidi Myrset, Fetsund (NO)

(73) Assignee: Nykode Therapeutics ASA, Oslo (NO)

(21) Appl. No.: 18/560,868

(22) PCT Filed: May 10, 2022
(86) PCT No.: PCT/EP2022/062688

§ 371 Date: Nov. 14, 2023

(30) Foreign Application Priority Data
May 10, 2021 (DK) PA 2021 70225
Jul. 8, 2021 (DK) PA 2021 70363
Jan. 26, 2022 (NO) 20220133

(51) Int. Cl. A61K 39/35; A61K 39/00; A61K 45/06; A61P 37/06; C12N 15/86

(52) U.S. Cl. CPC A61K 39/35; A61K 39/0008; A61K 45/06; A61P 37/06; C12N 15/86; C12N 2800/107; C12N 2800/60; C12N 2810/851; C12N 2840/206

(57) ABSTRACT

The present invention relates to vectors, such as DNA plasmids, comprising multiple nucleic acid sequences engineered to be co-expressed as separate molecules. Such separate molecules include a first polypeptide, wherein the first polypeptide comprises a targeting unit that targets antigen-presenting cells, a multimerization unit, such as dimerization unit, and an antigenic unit comprising one or more T cell epitopes of a self-antigen, an allergen, an alloantigen or a xenoantigen, and one or more immunoinhibitory compounds

### TECHNICAL FIELD

The present invention relates to vectors, such as DNA plasmids, comprising multiple nucleic acid sequences of interest engineered to be co-expressed as separate molecules, to pharmaceutical compositions comprising such vectors and to the use of such vectors and such pharmaceutical compositions in the treatment or prevention of diseases.

### BACKGROUND

Immune responses are necessary for protection against diseases, e.g. diseases caused by pathogens like viruses, bacteria or parasites. However, undesirable immune activation can cause processes leading to damage or destruction of one's own tissues. Undesirable immune activation occurs, for example, in autoimmune diseases where antibodies and/or T lymphocytes react with self-antigens, resulting in, e.g., tissue damage and pathology. Undesirable immune activation also occurs in allergic reactions, which are characterized by an exaggerated immune response to typically harmless substances in the environment, and which may result in inflammatory responses leading to tissue destruction. Further, undesired immune activation occurs in graft rejection, e.g. rejection of transplanted organs or tissue, which is significantly mediated by alloreactive T cells present in the host, which recognize donor alloantigens or xenoantigens, and which leads to destruction of the transplanted organ or tissue.

Immune tolerance is the acquired lack of specific immune responses to substances or tissue that have the capacity to elicit an immune response in a given organism.

Typically, to induce tolerance to a specific antigen, the antigen must be presented by an antigen-presenting cell (APC) to other immune cells in the absence of activation signal, which results in the death or functional inactivation of antigen specific lymphocytes or the generation of antigen-specific cells that maintain the tolerance. This process generally accounts for tolerance to self-antigens, or self-tolerance. Immunosuppressive drugs are useful in prevention or reduction of undesirable immune responses, e.g., in treating patients with autoimmune diseases or with allogeneic transplants. Conventional strategies for generating immunosuppression of an unwanted immune response are based on broad-acting immunosuppressive drugs.

Additionally, to maintain immunosuppression, immunosuppressive drug therapy is often a life-long proposition. Unfortunately, the use of broad-acting immunosuppressive drugs is associated with a risk of severe side effects, such as immunodeficiency, because the majority of them act non-selectively, resulting in increased susceptibility to infections and decreased cancer immunosurveillance. Accordingly, new compounds and compositions that induce antigen-specific tolerance would be beneficial.

Antigenpresenting cells (APCs), such as dendritic cells, play a key role in regulating the immune response, and, depending on the activation state and the microenvironment of the APC (cytokines and growth factors), it gives the antigen-specific T cells signals to either combat the presented antigens (presumed pathogens) or to silence the reaction to the presented antigen (presumed non-pathogenic antigens) and induce peripheral tolerance. The challenge in developing tolerogenic immunotherapies is to efficiently deliver the antigen to the APCs in a manner that does not trigger an inflammatory immune response.

The present invention relates to constructs that comprise an antigen unit comprising one or more T cell epitopes of a self-antigen, allergen, alloantigen or xenoantigen and a targeting unit, that interacts with surface molecules on APCs in a non-inflammatory or tolerogenic manner, which leads to the presentation of the antigen in the absence of an inflammatory activation status.

The Vaccibody construct is a dimeric fusion protein consisting of two polypeptides, each comprising a targeting unit, which targets antigen-presenting cells, a dimerization unit and an antigenic unit, which comprises one or more disease-relevant antigens or parts thereof. In another embodiment, the Vaccibody construct is a multimeric fusion protein consisting of multiple polypeptides, each comprising a targeting unit that targets APCs, a multimerization unit, and an antigenic unit that comprises one or more disease-relevant antigens or parts thereof—see for example WO 2004/076489 A1, WO 2011/161244 A1, WO 2013/092875 A1 or WO 2017/118695 A1. These constructs have shown to be efficient in generating an immune response against the antigens or parts thereof, e.g. epitopes, comprised in the antigenic unit.

The Vaccibody construct may be administered to a subject in the form of a polynucleotide encoding the polypeptide, e.g. a polynucleotide comprised in a vector, such as a DNA plasmid. After administration to host cells, e.g. administration to muscle cells of a subject, e.g. a human, the polypeptide is expressed and, due to the multimerization unit, such as dimerization unit, forms a multimeric fusion protein, such as a dimeric protein.

The present inventors have surprisingly found that a modified Vaccibody platform can be used to deliver disease-relevant antigens to APCs in an optimal way for the induction of an antigen-specific tolerance response of choice, through binding to and signalling through selected surface receptors on APCs that internalize the construct and present the antigens comprised therein in a tolerance inducing manner.

### SUMMARY

The present invention provides vectors, e.g. DNA plasmids, for co-expression of a construct and one or more immunoinhibitory compounds. The vector and pharmaceutical compositions comprising such vector is for use in the treatment of conditions involving undesirable immune reactions, such as in the prophylactic or therapeutic treatment of autoimmune diseases, allergic diseases and graft rejection.

Such a construct and immunoinhibitory compound(s) will, once the vector is administered to a subject, allow the presentation of the epitopes in the antigenic unit in a tolerance-inducing manner, and the vectors of the invention are thus suitable for use in the prophylactic or therapeutic treatment of immune diseases such as autoimmune diseases, allergic diseases and graft rejection.

As the construct and immunoinhibitory compound(s) cause downregulation of the disease-specific cells of the immune system causing the immune disease in question, they will not suppress the general immune system. Thus, treatment of the immune disease in question with the vectors of the invention will not result in increased susceptibility to infections and decreased cancer immunosurveillance.

The one or more immunoinhibitory compounds help to generate or promote an environment that favors the presentation of the epitopes in the antigenic unit in a tolerance-inducing manner, or by e.g. favoring the induction of tolerance maintaining cells or helping to maintain such cells.

In a first aspect, the present invention relates to a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of a self-antigen, an allergen, an alloantigen or a xenoantigen; and
(b) one or more further nucleic acid sequences encoding one or more immunoinhibitory compounds,
wherein the vector allows for the co-expression of the first polypeptide and the one or more immunoinhibitory compounds as separate molecules.

In one embodiment, the vectors of the invention comprise a first nucleic acid sequence encoding a first polypeptide, wherein the first polypeptide comprises a targeting unit that targets antigen-presenting cells without activating them.

The vectors of the invention may be used, e.g. in the form of pharmaceutical compositions, i.e. compositions comprising the vectors and a pharmaceutically acceptable carrier or diluent, for the prophylactic or therapeutic treatment of immune diseases by administering the vector/the pharmaceutical composition to a subject in need of such prophylactic or therapeutic treatment.

### DESCRIPTION OF DRAWINGS

FIG. 1

Shows an IRES co-expression element for use in the vector of the invention, which is inserted in between two coding regions. When the mRNA is formed, two ribosomes (T) are able to start translation at two separate sites on the mRNA and two proteins (A and B) are formed. A and B can for example be a first polypeptide and an immunoinhibitory compound.

FIG. 2

Shows a 2A self-cleaving peptide co-expression element for use in the vector of the invention, which is inserted between two genes. After transcription, one ribosome translates the mRNA and two proteins (A and B) are formed. Top of the figure shows how a fusion protein is formed if the 2A sequence is not part of the coding sequence. A and B can for example be a first polypeptide and an immunoinhibitory compound.

FIG. 3

Shows a bidirectional promoter (P) co-expression element for use in the vector of the invention, which is located between two coding regions. One mRNA is formed and two ribosomes (T) are able to start translation at two different mRNAs and two proteins (A and B) are formed. A and B can for example be a first polypeptide and an immunoinhibitory compound.

FIG. 4

Shows two promoters (P), i.e. co-expression elements for use in the vector of the invention, which are located before two coding regions. Two mRNAs are formed and two ribosomes (T) are able to start translation at two different mRNAs and two proteins (A and B) are formed. A and B can for example be a first polypeptide and an immunoinhibitory compound.

FIG. 5

Illustrates an embodiment of the construct encoded by the vector of the invention on the basis of the first polypeptide.

FIG. 6

Show the ELISA results (expression and secretion of proteins) obtained after transient transfection of HEK293 cells with the DNA vectors VB5049* (a vector according to the invention) or VB5052*.

FIG. 7

Show the results of a western blot analysis of the supernatant from Expi293F cells transfected with the DNA vectors VB5049* (a vector according to the invention) or VB5052*.

FIG. 8

Shows the results of a FluoroSpot assay (IL-10/IFN-γ ratio) for splenocytes which were obtained from mice after a single administration of either DNA vector VB5049* or VB5052* and re-stimulation with MOG (35-55) peptide.

FIG. 9

Shows the percentage of splenic IFN-γ+ T-cells (9A) and IL-17+ T-cells (9B) amongst the total CD4+ T cell population as determined upon 16 h re-stimulation with MOG (35-55) peptide as evaluated by flow cytometry. C57BL/6 mice were intramuscularly administered once with either DNA vector VB5049*, VB5051* or VB5052*, and the spleens were harvested after 7 days. Data were generated from a pool of 5 mice/group and the mouse spleens were pooled before analysis.

FIG. 10

Shows the results of a MOG tetramer staining and the detection of MOG-specific T cells. C57BL/6 mice were intramuscularly administered on day 0 with either DNA vector VB5049*, VB5051* or VB5052* followed by electroporation and spleens were harvested on day 7 post administration. The percentage of splenic Foxp3+ cells among the CD4+MOG (35-55) tet+ T-cell population was detected by H-2 IAb/MOG (35-55) tetramers. The tetramer staining was performed ex vivo and the splenocytes were not re-stimulated with MOG (35-55) peptide. Data were generated from a pool of 5 mice/group and the mouse spleens were pooled before analysis.

FIG. 11

Shows the protein expression and secretion levels of first polypeptides encoded by the indicated DNA vectors detected by sandwich ELISA (capture antibody: anti-MOG antibody, detection antibody: anti-hIgG CH3 domain antibody) with supernatant from Expi293F cells transiently transfected with said DNA vectors. Negative control (Neg ctrl): supernatant from Expi293F cells treated with the transfection reagent ExpiFectamine only.

FIG. 12

Show the expression and secretion levels of immunoinhibitory compounds encoded by the indicated DNA vectors detected by sandwich ELISA with supernatant from Expi293F cells transiently transfected with said DNA vectors. Negative control (Neg ctrl): supernatant from Expi293F cells treated with the transfection reagent ExpiFectamine only. The following antibodies were used: (A) Capture antibody: 1 μg/ml rat anti-murine IL-10 antibody, 100 μl/well, MAB417, R&D Systems. Detection antibody: 0.2 μg/ml, goat anti-murine IL-10 biotinylated antibody, 100 μl/well, BAF417, R&D Systems. (B) Capture antibody: 2 μg/ml TGF-beta 1 antibody, 100 μl/well, MAB2402, RD Systems. Detection antibody: 0.8 μg/ml chicken anti-human TGF-beta 1 biotinylated antibody, 100 μl/well, BAF240, RD Systems. (C) Capture antibody: 0.8 μg/ml goat anti-murine CTLA-4 antibody, 100 μl/well, AF476, RD Systems. Detection antibody: 0.8 μg/ml goat anti-murine CTLA-4 biotinylated antibody, 100 μl/well, BAF476, RD Systems. (D) Capture antibody: 2 μg/ml rat anti-murine IL-2 antibody, 100 μl/well, 503701, BioLegend. Detection antibody: 2 μg/ml rat anti-murine IL-2 biotinylated antibody, 100 μl/well, 503803, BioLegend. (E) Capture antibody: 2 μg/ml rat anti-murine IFN-γ antibody, 100 μl/well, 505802, BioLegend. Detection antibody: 2 μg/ml rat anti-murine IFN-γ biotinylated antibody, 100 μl/well, 505704, BioLegend.

FIG. 13

Show the separate expression and secretion of first polypeptides and immunoinhibitory compounds encoded by the indicated DNA vectors detected by sandwich ELISA with supernatant from Expi293F cells transiently transfected with said DNA vectors. Negative control (Neg ctrl): supernatant from Expi293F cells treated with the transfection reagent ExpiFectamine only. The following antibodies were used: capture antibody: mouse anti-MOG antibody, 0.25 μg/ml, 100 μl/well, sc-73330, Santa Cruz Biotechnology. Detection antibody: (A) 0.2 μg/ml goat anti-mouse anti-IL-10 biotinylated antibody, 100 μl/well, BAF417, R&D Systems. (B) 0.8 μg/ml chicken anti-human TGFβ1 biotinylated antibody, 100 μl/well, BAF240, RD Systems. (C) 0.8 μg/ml goat anti-mouse CTLA-4 biotinylated antibody, 100 μl/well, BAF476, RD Systems. (D) 2 μg/ml rat anti-mouse IFN-γ biotinylated antibody, 100 μl/well, 505704, BioLegend.

FIG. 14

Show the results of a western blot analysis of the supernatant from Expi293F cells transfected with the indicated DNA vectors. A: Reduced supernatant samples (35 μl loaded). Primary antibody: mouse anti-MOG (sc-73330). Secondary antibody: donkey anti-mouse, Dylight 800 (SA5-10172). Protein standard was detected in Chemidoc channel Dylight 650 (signal not shown). Chemidoc channels Dylight 800. Black arrow indicates the first polypeptide expressed from the respective DNA vector as a separate protein and secreted from the transfected cells. B: Non-reduced supernatant samples (35 μl loaded). Primary antibody: mouse anti-MOG (sc-73330). Secondary antibody: donkey anti-mouse, Dylight 800 (SA5-10172). Chemidoc channels Dylight 650 (for protein standard) and 800. Black arrow indicates the dimeric protein formed by two first polypeptide molecules expressed from the respective DNA vector and secreted from the transfected cells. C: Reduced supernatant samples (35 μl loaded). Primary antibody: rat anti-IL 10 (MAB417). Secondary antibody: donkey anti-rat, Dylight 488 (SA5-10026). Chemidoc channels Dylight 650 (for protein standard) and 488. Black arrow indicates IL-10 expressed as a separate protein from the respective DNA vector and secreted from the transfected cells. D: Reduced supernatant samples (35 μl loaded). Primary antibody: goat anti-CTLA-4 (AF476). Secondary antibody: donkey anti-goat, Dylight 800 (SA5-10092). Chemidoc channels Dylight 650 (for protein standard) and 800. Black arrow indicates CLTA-4 expressed as a separate protein from the respective DNA vector and secreted from the transfected cells. E: Reduced supernatant samples (35 μl loaded). Primary antibody: rat anti-IL2 (503702). Secondary antibody: donkey anti-rat, Dylight 650 (SA5-10029). Chemidoc channel Dylight 650. Black arrow indicates IL-2 expressed as a separate protein from the respective DNA vector and secreted from the transfected cells.

FIG. 15

Shows the expression and secretion of MOG (27-63) encoded by DNA vector VB5051 detected by sandwich ELISA with supernatant from Expi293F cells transiently transfected with said DNA vector. Negative control (Neg ctrl): supernatant from Expi293F cells treated with the transfection reagent ExpiFectamine only. Detection antibody: mouse anti-MOG antibody, 3.3 μg/ml, 100 μl/well, sc-73330, Santa Cruz Biotechnology.

FIG. 16

Show the results of a dual color IL-10/IFN-γ FluoroSpot assay. C57BL/6 mice were intramuscularly administered with the indicated DNA vectors four times (day 0, 3, 7 and 10) followed by electroporation and spleens were harvested on day 14 after the first administration. The splenocytes were tested for IL-10 and IFN-γ secretion (SFU/106 splenocytes) in a dual color FluoroSpot assay, non-re-stimulated (A) or upon 44 h re-stimulation with MOG (35-55) peptide (B). Individual mice and mean±SEM are shown, 5 mice/group, *(p<0.05), two-tailed Mann-Whitney test.

FIG. 17

Shows the IL-10/IFN-γ ratios calculated from data shown in FIG. 16B. Individual mice and mean±SEM are shown, 5 mice/group, *(p<0.05), two-tailed Mann-Whitney test.

FIG. 18

Shows the results of the detection of Foxp3+ producing CD4+ T cells by flow cytometry. C57BL/6 mice were administered with the indicated DNA vector followed by electroporation four times on days 0, 3, 7 and 10 and spleens were harvested on day 14 post first administration. The percentage of splenic CD4+Foxp3+ T cells was determined upon 16 h re-stimulation with MOG (35-55) peptide. Data were generated from a pool of 5 mice/group and the mouse spleens were pooled before analysis.

FIG. 19

Show the results of the detection of IFN-γ and IL-17 by flow cytometry. C57BL/6 mice were four times administered with the indicated DNA vectors followed by electroporation on days 0, 3, 7 and 10 and spleens were harvested on day 14 post first administration. The percentage of (A) IFN-γ+ T-cells and (B) IL-17+ T-cells amongst the total CD4+ T cell population was determined upon 16 h re-stimulation with MOG (35-55) peptide. Data were generated from a pool of 5 mice/group and the mouse spleens were pooled before analysis.

FIG. 20

Show the results of a dual color IL-10/IFN-γ FluoroSpot assay. C57BL/6 mice were intramuscularly administered on day 0 with the indicated DNA vectors followed by electroporation and spleens were harvested on day 7 post administration. The splenocytes were tested for IL-10 and IFN-γ secretion (SFU/106 splenocytes) with dual color FluoroSpot, non-re-stimulated (A) or upon 44 h re-stimulation with MOG (35-55) peptide (B). Individual mice and mean±SEM are shown, 5 mice/group, **(p<0.01), two-tailed Mann-Whitney test.

FIG. 21

Shows the IL-10/IFN-γ ratios calculated from data shown in FIG. 20B. Individual mice and mean±SEM are shown, 5 mice/group, *(p<0.05), two-tailed Mann-Whitney test.

FIG. 22

Shows the results of a MOG tetramer staining and the detection of MOG-specific T cells. C57BL/6 mice were intramuscularly administered on day 0 with the indicated DNA vectors followed by electroporation and spleens were harvested on day 7 post administration. The percentage of splenic Foxp3+ cells among the CD4+MOG (38-49) tet+ T-cell population was detected by H-2 IAb/MOG (38-49) tetramers. The tetramer staining was performed ex vivo and the splenocytes were not re-stimulated with MOG (35-55) peptide. Data were generated from a pool of 5 mice/group and the mouse spleens were pooled before analysis.

FIG. 23

Show the results of a dual color IL-10/IFN-γ FluoroSpot assay. C57BL/6 mice were intramuscularly administered on day 0 with the indicated DNA vectors followed by electroporation and spleens were harvested on day 7 post administration. The splenocytes were tested for IL-10 and IFN-γ secretion (SFU/106 splenocytes) with dual color FluoroSpot, non-re-stimulated (control, A) or upon 44 h re-stimulation with MOG (35-55) peptide (B). Individual mice and mean±SEM are shown, 5 mice/group, **(p<0.01), two-tailed Mann-Whitney test.

FIG. 24

Shows the IL-10/IFN-γ ratios calculated from data shown in FIG. 23B. Individual mice and mean±SEM are shown, 5 mice/group, *(p<0.01), two-tailed Mann-Whitney test.

FIG. 25

Shows the results of a MOG tetramer staining and the detection of MOG-specific T cells. C57BL/6 mice were intramuscularly administered on day 0 with the indicated DNA vectors followed by electroporation and spleens were harvested on day 7 post administration. The percentage of splenic Foxp3+ cells amongst the CD4+MOG (38-49) tet+ T-cell population was detected by H-2 IAb/MOG (38-49) tetramers. The tetramer staining was performed ex vivo and the splenocytes were not re-stimulated with MOG (35-55) peptide. Data were generated from a pool of 5 mice/group and the mouse spleens were pooled before analysis.

FIG. 26

Shows the results of the detection of proliferating Tregs. C57BL/6 mice were intramuscularly administered on day 0 with the indicated DNA vectors followed by electroporation and spleens were harvested on day 7 post administration. The percentage of Ki67+ cells amongst the Treg (CD4+CD25+Foxp3+) cell population was detected ex vivo. Data were generated from a pool of 5 mice/group and the mouse spleens were pooled before analysis.

FIG. 27

Show the results of a dual color IL-10/IFN-γ FluoroSpot assay. C57BL/6 mice were intramuscularly administered on day 0 with the indicated DNA vector followed by electroporation and spleens were harvested on day 7 post administration. The splenocytes were tested for IL-10 and IFN-γ secretion (SFU/106 splenocytes) with dual color FluoroSpot, non-re-stimulated (control, A) or upon 44 h re-stimulation with MOG (35-55) peptide (B). Individual mice and mean±SEM are shown, 5 mice/group, **(p<0.01), two-tailed Mann-Whitney test.

FIG. 28

Shows the IL-10/IFN-γ ratios calculated from data shown in FIG. 27B. Individual mice and mean±SEM are shown, 5 mice/group, *(p<0.01), two-tailed Mann-Whitney test.

FIG. 29

Shows the results of a MOG tetramer staining and the detection of MOG-specific T cells. C57BL/6 mice were intramuscularly administered on day 0 with the indicated DNA vector followed by electroporation and spleens were harvested on day 7 post administration. The percentage of splenic Foxp3+ cells amongst the CD4+MOG (38-49) tet+ T-cell population was detected by H-2 IAb/MOG (38-49) tetramers. The tetramer staining was performed ex vivo and the splenocytes were not re-stimulated with MOG (35-55) peptide. Data were generated from a pool of 5 mice/group and the mouse spleens were pooled before analysis.

FIG. 30

Shows the results of the detection of proliferating Tregs. C57BL/6 mice were intramuscularly administered on day 0 with the indicated DNA vectors followed by electroporation and spleens were harvested on day 7 post administration. The percentage of Ki67+ cells among the Treg (CD4+CD25+Foxp3+) cell population was detected ex vivo. Data were generated from a pool of 5 mice/group and the mouse spleens were pooled before analysis.

FIG. 31

Shows the expression and secretion of the first polypeptide encoded by the indicated DNA vectors detected by sandwich ELISA with supernatant from Expi293F cells transiently transfected with said DNA vectors. Negative control (Neg ctrl): supernatant from Expi293F cells treated with the transfection reagent ExpiFectamine only. Capture antibody: murine anti-MOG antibody, detection antibody: anti-hIgG CH3 domain antibody.

FIG. 32

Show the expression and secretion level of immunoinhibitory compounds encoded by the indicated DNA vectors detected by sandwich ELISA with supernatant from Expi293F cells transiently transfected with the said DNA vectors. Negative control (Neg ctrl): supernatant from Expi293F cells treated with the transfection reagent ExpiFectamine only. The following conditions and antibodies were used: A) Supernatant diluted 1:100. Capture antibody: mouse IL-10 antibody, 0.4 μg/ml, 100 μl/well, MAB417, R&D Systems. Detection antibody: mouse IL-10 biotinylated antibody, 0.2 pg/ml, BAF417, R&D Systems. B) Capture antibody: TGFβ1 antibody, 2 μg/ml, 100 μl/well, MAB2402, RD Systems. Detection antibody: chicken anti-TGFβ1 biotinylated antibody, 0.8 μg/ml, 100 μl/well, BAF240, R&D Systems. C) Capture antibody: mouse GM-CSF antibody, 2 μg/ml, 100 μl/well, MAB415, R&D Systems. Detection antibody: anti-GM-CSF Biotinylated antibody, 0.8 μg/ml, BAF415, R&D Systems.

FIG. 33

Show the separate expression and secretion of proteins encoded by the indicated DNA vectors detected by sandwich ELISA with supernatant from Expi293F cells transiently transfected with said DNA vectors. Negative control (Neg ctrl): supernatant from Expi293F cells treated with the transfection reagent ExpiFectamine only. Capture antibody: mouse anti-MOG antibody, 0.25 μg/ml, 100 μl/well, sc-73330, Santa Cruz Biotechnology. The following detection antibodies were used: A) mouse anti-IL-10 biotinylated antibody, 0.2 μg/ml, 100 μl/well, BAF417, R&D Systems, B) chicken anti-TGFβ1 biotinylated antibody, 0.8 μg/ml, 100 μl/well, BAF240, R&D Systems. C) anti-GM-CSF biotinylated antibody, 0.8 μg/ml, BAF415, R&D Systems.

FIG. 34

Show the results of a western blot analysis of the supernatant from Expi293F cells transfected with the indicated DNA vector. A: Reduced supernatant samples (35 μl loaded). Primary antibody: mouse anti-MOG (sc-73330). Secondary antibody: donkey anti-mouse, Dylight 800 (SA5-10172). Protein standard was detected in Chemidoc channel Dylight 650 (signal not shown). Chemidoc channels Dylight 800. Black arrow indicates the first polypeptide expressed from the respective DNA vector as a separate protein and secreted from the transfected cells. B: Non-reduced supernatant samples (35 μl loaded). Primary antibody: mouse anti-MOG (sc-73330). Secondary antibody: donkey anti-mouse, Dylight 800 (SA5-10172). Chemidoc channels Dylight 650 (for protein standard) and 800. Black arrow indicates the dimeric protein formed by two first polypeptide molecules expressed from the indicated DNA vector and secreted from the transfected cells. C: Reduced supernatant samples (35 μl loaded). Primary antibody: rat anti-IL10 (MAB417). Secondary antibody: donkey anti-rat, Dylight 488 (SA5-10026). Chemidoc channels Dylight 650 (for protein standard) and 488. Black arrow indicates IL-10 expressed as a separate protein from the respective DNA vector and secreted from the transfected cells. For VB5044 and VB5054, the increased size of IL-10 is due to the P2A tail fused to IL-10 as a result of ribosome skipping. D: Reduced supernatant samples (35 μl loaded). Primary antibody: rabbit anti-TGF-31 antibody (USB1042777-Biotin). Secondary antibody: donkey anti-rabbit, Dylight 650 (SA5-10041). Chemidoc channels Dylight 650. Black arrow indicates TGF-31 expressed as a separate protein from the respective DNA vector and secreted from the transfected cells. E: Reduced supernatant samples (35 μl loaded). Primary antibody: goat anti-murine GM-CSF (BAF415). Secondary antibody: donkey anti-goat, Dylight 800 (SA5-10092). Chemidoc channels Dylight 650 (for protein standard) and 800. Black arrow indicates GM-CSF expressed as a separate protein from the respective DNA vector and secreted from the transfected cells

FIG. 35

Shows the protein expression and secretion levels of the first polypeptide detected by sandwich ELISA (capture antibody: anti-MOG antibody, detection antibody: anti-hIgG CH3 domain antibody) with supernatant from Expi293F cells transiently transfected with DNA vector VB5068, VB5069 or VB5070. Negative control (Neg ctrl): supernatant from Expi293F cells treated with the transfection reagent ExpiFectamine only.

FIG. 36

Show the expression and secretion of the first polypeptides encoded by the indicated DNA vectors detected by sandwich ELISA (capture antibody: anti-MOG antibody, detection antibody: anti-hIgG CH3 domain antibody) with supernatant from Expi293F cells transiently transfected with said DNA vectors. Negative control (Neg ctrl): supernatant from Expi293F cells treated with the transfection reagent ExpiFectamine only. The following detection antibodies were used: A) anti-murine SCBG3A2 biotinylated antibody, 0.83 μg/ml, 100 μl/well, BAF3465, R&D Systems, B) anti-murine PD-1 biotinylated antibody, 0.72 μg/ml, 100 μl/ml, DY1021, R&D Systems).

FIG. 37

Shows the expression and secretion level of IL-10 encoded by the indicated DNA vectors detected by sandwich ELISA with supernatant from Expi293F cells transiently transfected with said DNA vectors. Negative control (Neg ctrl): supernatant from Expi293F cells treated with the transfection reagent ExpiFectamine only. Capture antibody: mouse IL-10 antibody, 0.4 μg/ml, 100 μl/well, MAB417, R&D Systems, detection antibody: mouse anti-IL-10 biotinylated antibody, 0.2 μg/ml, 100 μl/well, BAF417, R&D Systems

FIG. 38

Shows the separate expression and secretion of the immunoinhibitory compounds encoded by the indicated DNA vectors detected by sandwich ELISA with supernatant from Expi293F cells transiently transfected with said DNA vectors. Negative control (Neg ctrl): supernatant from Expi293F cells treated with the transfection reagent ExpiFectamine only. Supernatant dilution 1:100. Capture antibody: mouse anti-MOG antibody, 0.25 μg/ml, 100 μl/well, sc-73330, Santa Cruz Biotechnology, detection antibody: mouse anti-IL-10 biotinylated antibody, 0.2 μg/ml, 100 μl/well, BAF417, R&D Systems.

FIG. 39

Show the results of a western blot analysis of the supernatant from Expi293F cells transfected with the indicated DNA vectors. A: Reduced supernatant samples (35 μl loaded). Primary antibody: mouse anti-MOG (sc-73330). Secondary antibody: donkey anti-mouse, Dylight 800 (SA5-10172). Chemidoc channels Dylight 800. Black arrow indicates the intact first polypeptide expressed from the respective DNA vector as a separate protein and secreted from the transfected cells. B: Reduced supernatant samples (35 μl loaded). Primary antibody: rat anti-IL10 (MAB417). Secondary antibody: donkey anti-rat, Dylight 488 (SA5-10026). Chemidoc channels Dylight 650 (for protein standard) and 488. Black arrow indicates IL-10 expressed as a separate protein from the respective DNA vector and secreted from the transfected cells.

FIG. 40

Show the results of a dual color IL-10/IFN-γ FluoroSpot assay. C57BL/6 mice were intramuscularly administered on day 0 with the indicated DNA vector followed by electroporation and spleens were harvested on day 7 post administration. The splenocytes were tested for IL-10 and IFN-γ secretion (SFU/106 splenocytes) with dual color FluoroSpot, non-re-stimulated (control, A) or upon 44 h re-stimulation with MOG (35-55) peptide (B). Individual mice and mean±SEM are shown, 5 mice/group.

FIG. 41

Shows the results of a MOG tetramer staining and the detection of MOG-specific T cells. C57BL/6 mice were intramuscularly administered on day 0 with the indicated DNA vector followed by electroporation and spleens were harvested on day 7 post administration. The percentage Foxp3+ splenocytes among the CD4+MOG (38-49) tet+ splenocytes was detected by H-2 IAb/MOG (38-49) tetramers. The tetramer staining was performed ex vivo and the splenocytes were not re-stimulated with MOG (35-55) peptide. Data were generated from a pool of 5 mice/group and the mouse spleens were pooled before analysis.

FIG. 42

Shows the results of a MOG tetramer staining and the detection of MOG-specific T cells. C57BL/6 mice were intramuscularly administered on day 0 with the indicated DNA vector followed by electroporation and spleens were harvested on day 7 post administration. The percentage of MOG (38-49) tet+ splenocytes among the CD4+CD25+Foxp3+ splenocytes was detected by H-2 IAb/MOG (38-49) tetramers. The tetramer staining was performed ex vivo and the splenocytes were not re-stimulated with MOG (35-55) peptide. Data were generated from a pool of 5 mice/group and the mouse spleens were pooled before analysis.

FIG. 43

Shows the results of the detection of proliferating Tregs. C57BL/6 mice were intramuscularly administered on day 0 with the indicated DNA vectors followed by electroporation and spleens were harvested on day 7 post administration. The percentage of Ki67+ splenocytes among the Treg (CD4+CD25+Foxp3+) cell population was detected ex vivo. Data were generated from a pool of 5 mice/group and the mouse spleens were pooled before analysis.

FIG. 44

Show the results of the detection of CD4+CD25+Foxp3+ cells by flow cytometry. C57BL/6 mice were administered on day 0 with the indicated DNA vectors followed by electroporation and spleens were harvested on day 7 post administration. The percentage of CD4+CD25+Foxp3+ splenocytes was determined upon 16 h re-stimulation with MOG (35-55) peptide. Data were generated from a pool of 5 mice/group and the mouse spleens were pooled before analysis.

FIG. 45

Shows the expression and secretion of the first polypeptide encoded by the indicated DNA vector detected by sandwich ELISA (capture antibody: mouse anti-human IgG (CH3 domain), 1 μg/ml, 100 μl/well, MCA878G, BioRad, detection antibody: CaptureSelect™ Biotin Anti-IgG-Fc (Human) Conjugate, 1 μg/ml, 100 μl/well, 7103262100, Thermo Fisher) with supernatant from Expi293F cells transiently transfected with said DNA vector. Negative control (Neg ctrl): supernatant from Expi293F cells treated with the transfection reagent ExpiFectamine only.

FIG. 46

Shows the expression and secretion level of IL-10 encoded by the indicated DNA vector detected by sandwich ELISA with supernatant from Expi293F cells transiently transfected with the said DNA vector. Negative control (Neg ctrl): supernatant from Expi293F cells treated with the transfection reagent ExpiFectamine only. Capture antibody: mouse IL-10 antibody, 0.4 μg/ml, 100 μl/well, MAB417, R&D Systems. Detection antibody: mouse IL-10 biotinylated antibody, 0.2 μg/ml, BAF417, R&D Systems.

FIG. 47

Show the results of a western blot analysis of the supernatant from Expi293F cells transfected with the indicated DNA vector. Reduced supernatant samples (35 μl loaded). Primary antibody: rat anti-IL-10 (MAB417). Secondary antibody: donkey anti-rat, Dylight 650 (SA5-10029). Chemidoc channel Dylight 650. Black arrow indicates IL-10 expressed as a separate protein from the indicated DNA vector and secreted from the transfected cells.

### DETAILED DESCRIPTION

The first polypeptide and/or the multimeric protein will herein also be referred to as a construct.

In one embodiment, the construct is a tolerance-inducing construct.

A “tolerance-inducing construct” is one that does not elicit an inflammatory immune response but rather does induce tolerance towards the T cell epitopes comprised in the antigenic unit, when administered to a subject in a form suitable for administration and in an amount effective to induce tolerance (i.e. an effective amount).

The term “tolerance” as used herein refers to a decreased level of an inflammatory immune response, a delay in the onset or progression of an inflammatory immune response and/or a reduced risk of the onset or progression of an inflammatory immune response towards antigens like self-antigens, allergens or alloantigens or xenoantigens.

A “subject” is an animal, e.g. a mouse, or a human, preferably a human. The terms “mouse”, “murine” and “m” are used interchangeably herein to denote a mouse or refer to a mouse. The terms human and “h” are used interchangeably herein to denote a human or refer to a human. A subject may be a patient, i.e. a human suffering from an immune disease, like an autoimmune disease, an allergy, or a graft rejection, who is in need of a therapeutic treatment or it may be a subject in need of prophylactic treatment or a subject suspected of having an immune disease. The terms “subject” and “individual” are used interchangeably herein.

A “disease” is an abnormal medical condition that is typically associated with specific signs and symptoms in a subject being affected by the disease. An “immune disease” as used herein refers to conditions involving undesired immune reactions, including autoimmune diseases, allergies or a graft rejection, i.e. rejection of allografts or xenografts such as rejection by a host of cells, tissue or organs from the same (allo) or a different (xeno) species transplanted to the host.

A “treatment” is a prophylactic treatment or therapeutic treatment.

A “prophylactic treatment” is a treatment administered to a subject who does not display signs or symptoms of, or displays only early signs or symptoms of, an immune disease, such that treatment is administered for the purpose of preventing or decreasing the risk of developing the immune disease. A prophylactic treatment functions as a preventative treatment against an immune disease, or as a treatment that inhibits or reduces further development or enhancement of the immune disease and/or its associated symptoms. The terms prophylactic treatment, prophylaxis and prevention are used interchangeably herein.

A “therapeutic treatment” is a treatment administered to a subject who displays symptoms or signs of an immune disease, in which treatment is administered to the subject for the purpose of diminishing or eliminating those signs or symptoms or for the purpose of delaying or stopping disease progression.

A “T cell epitope” as used herein refers to a discrete, single T cell epitope or a part or region of an antigen containing multiple T cell epitopes, e.g. multiple minimal T cell epitopes, such as a hotspot.

A “nucleotide sequence” is a sequence consisting of nucleotides. The terms “nucleotide sequence” and “nucleic acid sequence” are used interchangeably herein.

The section headings used herein are for organizational purposes only and are not to be construed as limiting the subject matter described.

### Vector

The vectors of the invention may be any molecules which are suitable to carry foreign nucleic acid sequences, such as DNA or RNA, into a cell, where they can be expressed, i.e. expression vectors.

In one embodiment, the vector is a DNA vector, such as a DNA plasmid or a DNA viral vector, such as a DNA viral vector selected from the group consisting of adenovirus, vaccinia virus, adeno-associated virus, cytomegalovirus and Sendai virus.

In another embodiment, the vector is an RNA vector, such as an RNA plasmid or an RNA viral vector, such as a retroviral vector, e.g. a retroviral vector selected from the group consisting of alphavirus, lentivirus, Moloney murine leukemia virus and rhabdovirus.

In a preferred embodiment, the vector is a DNA vector, more preferably a DNA plasmid.

### DNA Plasmids

A plasmid is a small, extrachromosomal DNA molecule within a cell that is physically separated from chromosomal DNA and can replicate independently. Plasmids are mostly found as small circular, double-stranded DNA molecules in bacteria; however, plasmids are sometimes present in archaea and eukaryotic organisms. Artificial plasmids are widely used as vectors in molecular cloning, serving to deliver and ensure high expression of recombinant DNA sequences within host organisms. Plasmids comprise several important features, including a feature for selection of cells comprising the plasmid, such as for example a gene for antibiotic resistance, an origin of replication, a multiple cloning site (MCS) and promoters for driving the expression of the inserted gene(s) of interest.

Generally, promoters are sequences capable of attracting initiation factors and polymerases to the promoter, so that a gene is transcribed. Promoters are located near the transcription start sites of genes, upstream on the DNA. Promoters can be about 100-1000 base pairs long. The nature of the promoter is usually dependent on the gene and product of transcription and type or class of RNA polymerase recruited to the site. When the RNA polymerase reads the DNA of the plasmid, an RNA molecule is transcribed. After processing, the mRNA will be able to be translated numerous times, and thus result in many copies of the proteins encoded by the genes of interest, when the ribosome translates the mRNA into protein. Generally, the ribosome facilitates decoding by inducing the binding of complementary tRNA anticodon sequences to mRNA codons. The tRNAs carry specific amino acids that are chained together into a polypeptide as the mRNA passes through and is “read” by the ribosome. Translation proceeds in three phases, initiation, elongation, and termination. Following the translation process, the polypeptide folds into an active protein and performs its functions in the cell or is exported from the cell and performs its functions elsewhere, sometimes after a considerable number of posttranslational modifications.

When a protein is destined for export out of the cell, a signal peptide directs the protein into the endoplasmic reticulum, where the signal peptide is cleaved off and the protein is transferred to the cell periphery after translation has terminated.

The DNA plasmid is not limited to any specific plasmid, the skilled person will understand that any plasmid with a suitable backbone can be selected and engineered by methods known in the art to comprise the elements and units of the present disclosure.

### Co-Expression

The vectors of the present disclosure co-express several proteins. Such vectors (and plasmids) are also referred to as multicistronic or polycistronic vectors (and multicistronic or polycistronic plasmids). The skilled person knows how to engineer a vector to comprise sequences coding for these several proteins and can select different techniques, so that these proteins are co-expressed from one vector as separate proteins.

Hence, the skilled person can construct the vectors of the invention, co-expressing different proteins, i.e. a first polypeptide and one or more immunoinhibitory compounds.

In a preferred embodiment, the vectors of the invention comprise one or more co-expression elements, i.e. nucleic acid sequences which allow for co-expression of the first polypeptide and the one or more immunoinhibitory compounds from the same vector.

In one embodiment of the present disclosure, the vector comprises a co-expression element (or more than one co-expression element), which causes that the first polypeptide and the one or more immunoinhibitory compounds are transcribed on a single transcript but independently translated into the first polypeptide and the one or more immunoinhibitory compounds. Hence, the presence of the co-expression element results in a final production of separate translation products.

### IRES

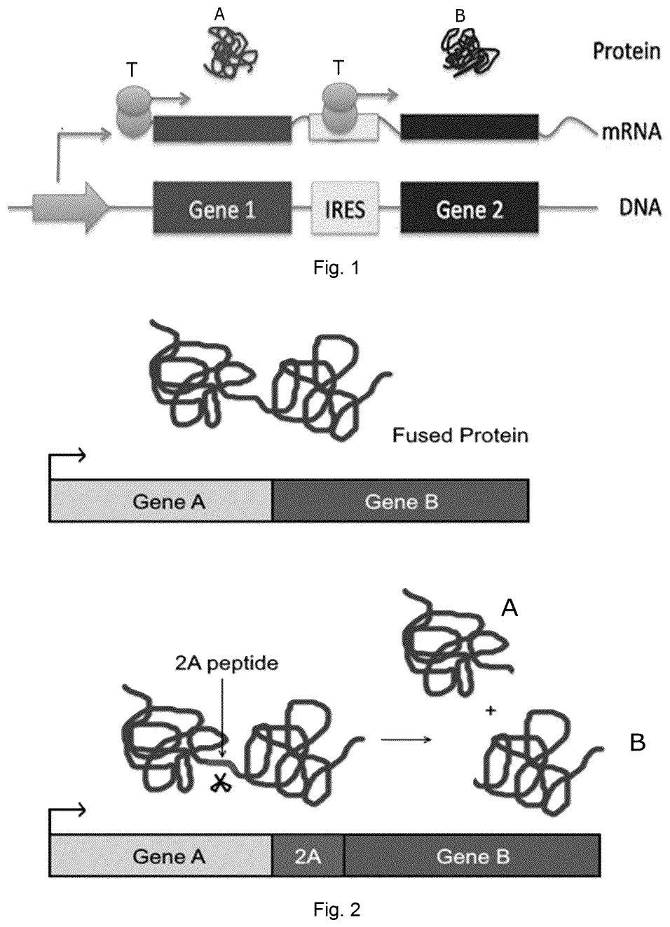

In one embodiment of the present disclosure, the co-expression element is an IRES element, the concept of which is illustrated in FIG. 1. An internal ribosome entry site, abbreviated IRES, is an RNA element that allows for translation initiation in a cap-independent manner, as part of the greater process of protein synthesis. In eukaryotic translation, initiation typically occurs at the 5′ end of mRNA molecules, since 5′ cap recognition is required for the assembly of the initiation complex. By placing an IRES element between two coding regions, the initiation complex can be assembled at this site and allow for translation of the downstream coding region. Hence, in an embodiment of the present disclosure, the vector comprises an IRES and one transcript is produced from the vector, which subsequently is translated into separate proteins.

The IRES element allows the co-expression of the first polypeptide and the one or more immunoinhibitory compounds under the control of the same promoter. The promoter directs the transcription of a single mRNA containing coding regions for the nucleic acid sequence encoding the first polypeptide and the nucleic acid sequences encoding the one or more immunoinhibitory compounds. If more than one immunoinhibitory compound is expressed from the vector of the invention, an IRES element needs to be present in the vector of the invention upstream of each nucleic acid sequence encoding an immunoinhibitory compound. Alternatively, another type of co-expression element may be used if more than one immunoinhibitory compound is expressed from the vector of the invention.

The IRES elements for use in the vector of the invention may be derived from viral genomes or from cellular mRNA. Vectors comprising IRES elements, such as DNA plasmids, are commercially available.

### 2A Self-Cleaving Peptides

In another embodiment of the present disclosure, the co-expression element is a nucleic acid sequence encoding a 2A self-cleaving peptide (or short “2A peptide”), the concept of which is illustrated in FIG. 2.

In the context of this application, the terms “2A self-cleaving peptide” and “2A peptide” are used for a peptide encoded by a nucleic acid sequence that, when positioned between two coding regions, cause the transcription of the two coding regions as a single transcript, but its translation into two separate peptide chains. Generally, when the ribosome translates mRNA, amino acids are covalently bonded in an N-terminal to C-terminal fashion. The presence of a nucleic acid sequence encoding a 2A self-cleaving peptide results in two separate peptide chains because the ribosome skips the synthesis of a peptide bond at the C-terminus of the 2A peptide. 2A self-cleaving peptides are typically 18-22 amino acids long and often comprise the consensus sequence DXEXNPGP (SEQ ID NO: 68), wherein X can be any amino acid.

In one embodiment of the present invention, the ribosome skips the peptide bond between a glycine and a proline residue found on the C-terminus of the 2A self-cleaving peptide, meaning that the upstream gene product will have a few additional amino acid residues added to the end, while the downstream gene product will start with a proline.

In one embodiment, the 2A self-cleaving peptide is an 18-22 amino acid long sequence comprising the consensus sequence DXEXNPGP (SEQ ID NO: 68), wherein X can be any amino acid.

Thus, also the 2A self-cleaving peptide allows for the co-expression of the first polypeptide and the one or more immunoinhibitory compounds under the control of the same promoter. As with the IRES element, if more than one immunoinhibitory compound is expressed from the vector of the invention, a nucleic acid sequence encoding a 2A peptide needs to be present in the vector upstream of each nucleic acid sequence encoding an immunoinhibitory compound. As an example, the vector comprises a first nucleic acid sequence encoding a first polypeptide, a second nucleic acid sequence encoding a first immunoinhibitory compound and a third nucleic acid sequence encoding a second immunoinhibitory compound. The vector may comprise a nucleic acid sequence encoding a T2A peptide between the first and the second nucleic acid sequence and a nucleic acid sequence encoding a P2A peptide between the second and the third nucleic acid sequence. Alternatively, another type of co-expression element may be used if more than one immunoinhibitory compound is expressed from the vector of the invention.

In a further embodiment, the 2A self-cleaving peptide is a 2A-peptide selected from the group consisting of T2A peptide, P2A peptide, E2A peptide and F2A peptide.

In one embodiment, the T2A peptide has an amino acid sequence identical to the T2A sequences listed in Table 1 or 2. In a further embodiment, the amino acid sequence DVEENPGP (SEQ ID NO: 69) is present but the remainder of the T2A amino acid sequence has 80% to 100% sequence identity to the T2A amino acid sequence of Table 1, such as 81%, 82%, 83%, 84%, 85%, 86%, 87%, 88%, 89%, 90%, 91%, 92%, 93%, 94%, 95%, 96%, 97%, 98% or 99% sequence identity. In another embodiment, the T2A peptide has the amino acid sequence with SEQ ID NO: 6.

In one embodiment, the P2A peptide has an amino acid sequence identical to the P2A sequence listed in Table 1 or 2. In a further embodiment, the amino acid sequence DVEENPGP (SEQ ID NO: 69) is present but the remainder of the P2A amino acid sequence has 80% to 100% sequence identity to the P2A amino acid sequence of Table 1, such as 81%, 82%, 83%, 84%, 85%, 86%, 87%, 88%, 89%, 90%, 91%, 92%, 93%, 94%, 95%, 96%, 97%, 98% or 99% sequence identity. In another embodiment, the P2A peptide has the amino acid sequence with SEQ ID NO: 7.

In one embodiment, the E2A peptide has an amino acid sequence identical to the E2A sequence listed in Table 1 or 2. In a further embodiment, the amino acid sequence DVESNPGP (SEQ ID NO: 70) is present but the remainder of the E2A amino acid sequence has 80% to 100% sequence identity to the E2A sequence of Table 1, such as 81%, 82%, 83%, 84%, 85%, 86%, 87%, 88%, 89%, 90%, 91%, 92%, 93%, 94%, 95%, 96%, 97%, 98% or 99% sequence identity. In another embodiment, the E2A peptide has the amino acid sequence with SEQ ID NO: 8.

In one embodiment, the F2A peptide has an amino acid sequence identical to the F2A sequence listed in Table 1 or 2. In a further embodiment, the amino acid sequence DVESNPGP (SEQ ID NO: 70) is present but the remainder of the F2A amino acid sequence has 80% to 100% sequence identity to the F2A sequence of Table 1, such as 81%, 82%, 81%, 82%, 83%, 84%, 85%, 86%, 87%, 88%, 89%, 90%, 91%, 92%, 93%, 94%, 95%, 96%, 97%, 98% or 99% sequence identity. In another embodiment, the F2A peptide has the amino acid sequence with SEQ ID NO: 9.

TABLE 1
Sequences of 2A self-cleaving peptides

| Name | Sequence | SEQ ID NO. |
|---|---|---|
| T2A | EGRGSLLTCGDVEENPGP | SEQ ID NO: 6 |
| P2A | ATNFSLLKQAGDVEENPGP | SEQ ID NO: 7 |
| E2A | QCTNYALLKLAGDVESNPGP | SEQ ID NO: 8 |
| F2A | VKQTLNFDLLKLAGDVESNPGP | SEQ ID NO: 9 |

It is generally known that the efficiency of the 2A-peptides can be modulated to increase their efficiency in cleavage and expression, for example by inserting a GSG sequence prior to the N-terminus of the wildtype sequences, as shown in Table 2.

TABLE 2
Further sequences of 2A self-cleaving peptides

| Name | Sequence | SEQ ID NO. |
|---|---|---|
| T2A | GSGEGRGSLLTCGDVEENPGP | SEQ ID NO: 71 |
| P2A | GSGATNFSLLKQAGDVEENPGP | SEQ ID NO: 72 |
| E2A | GSGQCTNYALLKLAGDVESNPGP | SEQ ID NO: 73 |
| F2A | GSGVKQTLNFDLLKLAGDVESNPGP | SEQ ID NO: 74 |

In another embodiment, the vector of the invention contains both IRES elements and nucleic acid sequences encoding 2A peptides. As an example, the vector comprises a first nucleic acid sequence encoding a first polypeptide, a second nucleic acid sequence encoding a first immunoinhibitory compound and a third nucleic acid sequence encoding a second immunoinhibitory compound. The vector may comprise an IRES element between the first and the second nucleic acid sequence and a nucleic acid sequence encoding a 2A peptide between the second and the third nucleic acid sequence. Alternatively, the vector may comprise a nucleic acid sequence encoding a 2A peptide between the first and the second nucleic acid sequence and an IRES element between the second and the third nucleic acid sequence. Further nucleic acid sequences encoding further immunoinhibitory compounds may be included in the vector in the same manner.

In another embodiment, the vector of the invention contains nucleic acid sequences encoding two 2A peptides, i.e. as a continuous sequence consisting of two 2A peptides. As an example, the vector comprises a first nucleic acid sequence encoding a first polypeptide and a second nucleic acid encoding an immunoinhibitory compound. The vector may comprise a nucleic acid sequence encoding two 2A peptides as a continuous sequence between the first and the second nucleic acid sequence.

### Bidirectional Promoters

In one embodiment of the present disclosure, the vector comprises a co-expression element (or more than one co-expression element) which causes that the first polypeptide and the one or more immunoinhibitory compounds are transcribed as separate transcripts, which results in separate transcription products and thus separate proteins.

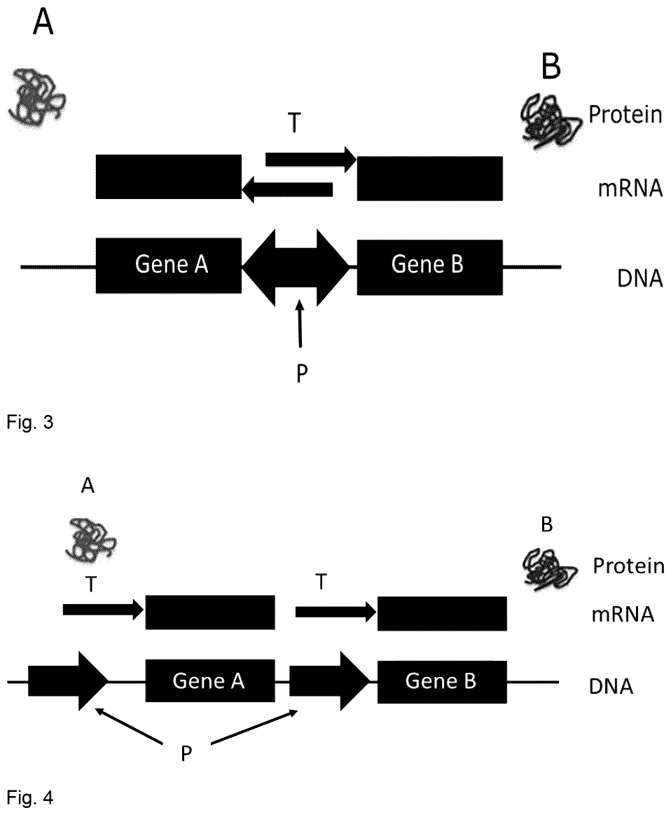

In one embodiment of the present disclosure, the co-expression element is a bidirectional promoter, the concept of which is illustrated in FIG. 3. Bidirectional promoters are typically short (e.g. <1 kbp) intergenic regions of DNA between the 5′ ends of the genes in a bidirectional gene pair. A “bidirectional gene pair” refers to two adjacent genes coded on opposite strands, with their 5′ ends oriented toward one another.

In one embodiment of the present disclosure, the bidirectional promoter is a back-to-back arrangement of CAG promoters with four CMV enhancers (Sladitschek H L, Neveu P A et al. PLOS One 11(5), e0155177, 2016).

In one embodiment of the present disclosure, the bidirectional promoter is RPBSA (Kevin He et al, Int. J. Mol. Sci. 21(23), 9256, 2020).

In one embodiment of the present disclosure, the bidirectional promoter is a back-to-back configuration of the mouse Pgk1 and human eukaryotic translation elongation factor 1 alpha 1 promoters (Golding & Mann, Gene Therapy 18, 817-826, 2011).

In one embodiment, the vector of the invention is a plasmid which comprises a first nucleic acid sequence encoding the first polypeptide and a second nucleic acid sequence encoding an immunoinhibitory compound as a bidirectional gene pair comprising between their 5′ ends a bidirectional promoter.

### Multiple Promoters

In another embodiment of the present disclosure, the co-expression elements are various promoters, i.e. the vector is e.g. a plasmid which comprises a separate promoter for each of the nucleic acid sequences encoding the first polypeptide and the one or more immunoinhibitory compounds, i.e. for separate transcription of the first polypeptide and each of the one or more immunoinhibitory compounds.

In one embodiment, each of said nucleic acid sequence will have a different promoter, the concept of which is illustrated in FIG. 4. In one embodiment, all nucleic acid sequences have the same promoter to aim at equimolecular expression. In an alternative embodiment, one nucleic acid sequence has a stronger promoter than the other(s); that is, the nucleic acid sequence with a stronger promoter is likely to be expressed at higher levels than the other(s).

Numerous promoters are known in the art and suitable for inclusion into the plasmids of the invention. In one embodiment of the present disclosure, the promoter is derived from cytomegalovirus, such as the CMV promoter.

### Combination of Different Co-Expression Elements

In one embodiment, the vector of the invention comprises one or more co-expression elements, preferably co-expression elements selected from the group consisting of IRES element, 2A peptide, bidirectional promoter and promoter.

The vector of the invention may comprise all kinds of combinations of co-expression elements.

As an example, the vector of the invention is a DNA plasmid which comprises a first nucleic acid sequence encoding a first polypeptide, a second nucleic acid sequence encoding a first immunoinhibitory compound and a third nucleic acid sequence encoding a second immunoinhibitory compound. In one embodiment, the DNA plasmid comprises an IRES and a 2A peptide which allows the co-expression of the first polypeptide (under control of a promoter) and of the first and second immunoinhibitory compound. In another embodiment, the DNA plasmid comprises a bidirectional promoter and another promoter.

The skilled person will know that the terms first-second, and third nucleic acid sequences as in the example above does not mean that the plasmid of the invention comprises the nucleic acid sequences in the order of first, second and third nucleic acid sequence. The second nucleic acid sequence may be downstream or upstream of the first or third nucleic acid sequence, the third nucleic acid sequence may be downstream or upstream of the first or second nucleic acid sequence and the first nucleic acid sequence may be upstream or downstream of the second or third nucleic acid sequence. In another embodiment, the first- and the second nucleic acid sequences might be in opposite directions on the same DNA strand, as may be the first and third or the second and third nucleic acid sequences. In further embodiments, the nucleic acid sequences encoding the first polypeptide and the immunoinhibitory compounds might be on opposite DNA strands.

### Immunoinhibitory Compounds

The vectors of the present invention comprise one or more nucleic acid sequences encoding one or more immunoinhibitory compounds.

In one embodiment of the present disclosure, the immunoinhibitory compound is a compound that induces, increases or maintains immune tolerance. In another embodiment of the present disclosure, the immunoinhibitory compound is a compound that is known to induce, increase or maintain immune tolerance.

In yet another embodiment, the immunoinhibitory compound is a compound that favors the presentation of the epitopes in the antigenic unit in a tolerance inducing manner, and/or that favors the induction of tolerance maintaining cells (regulatory T cells, not only anergy or apoptosis of the effector T cells) and/or that helps maintaining such tolerance maintaining cells.

In yet another embodiment, the immunoinhibitory compound is a compound that promotes and/or supports the presentation of the epitopes in the antigenic unit in a tolerance inducing manner, and/or that promotes and/or supports the induction of tolerance maintaining cells (regulatory T cells, not only anergy or apoptosis of the effector T cells) and/or that helps maintaining such tolerance maintaining cells

In one embodiment of the present disclosure, the immunoinhibitory compound is an extracellular part, such as the extracellular domain, of an inhibitory checkpoint molecule. In one embodiment, the inhibitory checkpoint molecule is selected from the group consisting of CLTA-4, PD-1, BTLA, LAG3, NOX2, SIGLEC7, SIGLEC9 and TIM-3. In one embodiment, the inhibitory checkpoint molecule is CLTA-4. In one embodiment, the inhibitory checkpoint molecule is PD-1. In one embodiment, the inhibitory checkpoint molecule is BTLA. In one embodiment, the inhibitory checkpoint molecule is TIM-3.

In a preferred embodiment, the immunoinhibitory compound is an extracellular part, such as the extracellular domain, of a human (h) inhibitory checkpoint molecule, such as an extracellular part, such as the extracellular domain, of a human inhibitory checkpoint molecule selected from the group consisting of hCLTA-4, hPD-1, hBTLA, hLAG3, hNOX2, hSIGLEC7, hSIGLEC9 and hTIM-3. In one embodiment, the inhibitory checkpoint molecule is hCLTA-4, such as hCTLA-4 with SEQ ID NO: 51. In one embodiment, the inhibitory checkpoint molecule is hPD-1, such as hPD-1 with SEQ ID NO: 52. In one embodiment, the inhibitory checkpoint molecule is hBTLA. In one embodiment, the inhibitory checkpoint molecule is hTIM-3

In one embodiment of the present disclosure, the immunoinhibitory compound is a cytokine selected from the group consisting of IL-10, TGF-β1, TGF-β2, TGF-β3, IL-27, IL-2, GM-CSF, FLT3L, IFN-γ, IL-37 and IL-35. In one embodiment, the cytokine is IL-10. In one embodiment, the cytokine is TGF-β1. In one embodiment, the cytokine is IL-27. In one embodiment, the cytokine is IL-2. In one embodiment, the cytokine is GM-CSF. In one embodiment, the cytokine is FLT3L. In one embodiment, the cytokine is IFN-γ. In one embodiment, the cytokine is IL-37. In one embodiment, the cytokine is IL-35.

In a preferred embodiment, the immunoinhibitory compound is a human cytokine selected from the group consisting of hIL-10, hTGF-β1, hTGF-β2, hTGF-β3, hIL-27, hIL-2, hGM-CSF, hFLT3L, hIFN-γ, hIL-37 and hIL-35. In one embodiment, the cytokine is hIL-10, such as hIL-10 with SEQ ID NO: 53. In one embodiment, the cytokine is hTGF-β1, such as hTGF-β1 with SEQ ID NO: 54. In one embodiment, the cytokine is hTGF-β2, such as hTGF-β2 with SEQ ID NO: 58. In one embodiment, the cytokine is hTGF-β3, such as hTGF-β3 with SEQ ID NO: 59. In one embodiment, the cytokine is hIL-27. In one embodiment, the cytokine is hIL-2, such as hIL-2 with SEQ ID NO: 55. In one embodiment, the cytokine is hGM-CSF, such as hGM-CSF with SEQ ID NO: 56. In one embodiment, the cytokine is hFLT3L. In one embodiment, the cytokine is hIFN-γ, such as hIFN-γ with SEQ ID NO: 57. In one embodiment, the cytokine is hIL-37. In one embodiment, the cytokine is hIL-35.

### Vectors Comprising Multiple Nucleic Acid Sequences Encoding Multiple Immunoinhibitory Compound

In one embodiment of the present disclosure, the vector comprises nucleic acid sequences encoding 2, 3, 4, 5, 6, 7 or 8 immunoinhibitory compounds. In another embodiment, the vector comprises nucleic acid sequences encoding 2 to 6 immunoinhibitory compounds, i.e. 2 or 3 or 4 or 5 or 6 immunoinhibitory compounds. The immunoinhibitory compounds may be the same or different, preferably different.

In a preferred embodiment, the different immunoinhibitory compounds generate or promote a tolerance-inducing environment on many different levels. By way of example, the vector of the invention comprises nucleic acid sequences encoding 3 different immunoinhibitory compounds, wherein the first induces tolerance, the second increases tolerance and the third maintains tolerance.

### The First Nucleic Acid Sequence

The vectors of the present disclosure comprise a first nucleic acid sequence, i.e. a DNA or RNA, including genomic DNA, cDNA and mRNA, either double-stranded or single-stranded, which encodes a first polypeptide. In one embodiment, the first nucleic acid sequence is a DNA. In another embodiment, the first nucleic acid sequence is optimized to the species of the subject to which it is administered. For administration to a human, in one embodiment, the first nucleic acid sequence is human codon optimized.

The first nucleic acid sequence encodes a first polypeptide comprising a targeting unit that targets APCs, a multimerization unit, such as dimerization unit, and an antigenic unit, wherein the antigenic unit comprises one or more T cell epitopes of a self-antigen, an allergen, an alloantigen or a xenoantigen. Once administered to a subject, the first polypeptide is expressed and, due to the presence of the multimerization unit, forms a multimeric protein, which elicits a tolerogenic response to the one or more T cell epitopes comprised in the antigenic unit.

### Construct

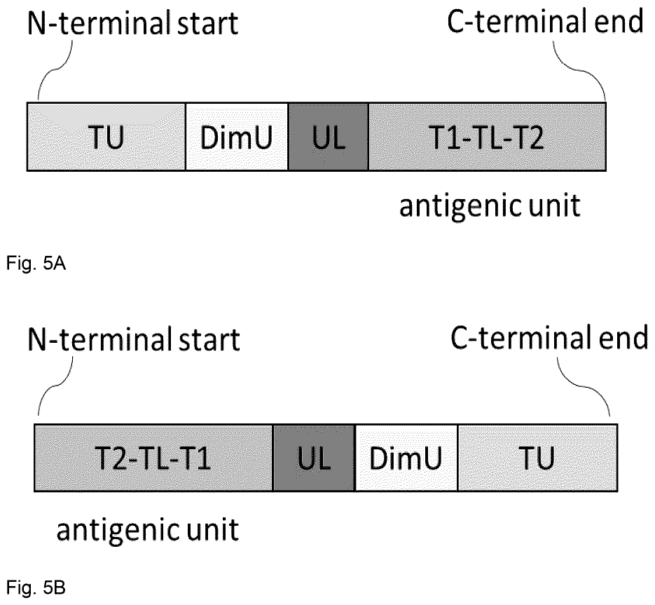

The construct can be described as a polypeptide having an N-terminal start and a C-terminal end (illustrated in FIG. 5). The elements and units of the first polypeptide—targeting unit (TU), multimerization unit, such as, in this FIG. 5, a dimerization unit (DimU), and antigenic unit—may be arranged in the polypeptide such that the antigenic unit is located at the C-terminal end of the polypeptide (FIG. 5a) or at the N-terminal start of the polypeptide (FIG. 5b). Preferably, the antigenic unit is located at the C-terminal end of the polypeptide.

The antigenic unit comprises one or more T cell epitope(s) and, if multiple T cell epitopes are present, may comprise one or more T cell epitope linkers to separate the T cell epitopes. A unit linker (UL) may connect the multimerization unit, such as a dimerization unit, and the antigenic unit. FIG. 5 illustrates an antigenic unit with 2 T cell epitopes (T1, T2), which are separated by a T cell epitope linker (TL). The order and orientation of the above-described units and elements is the same in the multimeric protein and the first nucleic acid sequence encoding the first polypeptide.

In the following, the various units and elements of the first polypeptide/construct will be discussed in detail. They are present in the first nucleic acid sequence as nucleic acid sequences encoding the units/elements while they are present in the first polypeptide or multimeric protein as amino acids sequences. For the ease of reading, in the following, the units/elements are mainly explained in relation to the first polypeptide/multimeric protein, i.e. on the basis of their amino acid sequences.

### Targeting Unit

The first polypeptide encoded by the first nucleic acid comprised in the vectors of the invention comprises a targeting unit that targets APCs.

The term “targeting unit” as used herein refers to a unit that delivers the construct of the invention to an antigen-presenting cell and interacts with surface molecules on the APC, e.g. binds to surface receptors on the APC, without activating the cell.

In another embodiment, the term “targeting unit” as used herein refers to a unit that delivers the construct of the invention to an antigen-presenting cell and interacts with surface molecules on the APC, e.g. binds to surface receptors on the APC, without inducing maturation of the cell.

The APC internalizes the construct and presents the T cell epitopes comprised in the antigenic unit on MHC on its surface in an anti-inflammatory, tolerogenic manner, e.g. by not upregulating co-stimulatory signals and/or by upregulating inhibitory surface molecules and/or by promoting the secretion of inhibitory cytokines.

In one embodiment, the targeting unit comprises or consists of a moiety that binds to a surface molecule on APCs selected from the group consisting of TGFβ receptor (including TGFβR1, TGFβR2, and TGFβR3), IL-10R, such as IL-10RA and IL-10RB, IL-2R, IL-4R, IL-6R, IL-11R, IL-13R, IL-27R, IL-35R, IL-37R, GM-CSFR, FLT3, CCR7, CD11b, CD11c, CD103, CD14, CD36, CD205, CD109, VISTA, MARCO, MHCII, CD83, SIGLEC, Clec10A (MGL), ASGR (ASGR1/ASGR2), CD80, CD86, Clec9A, Clec12A, Clec12B, DCIR2, Langerin, MR, DC-Sign, Treml4, Dectin-1, PDL1, PDL2, HVEM, CD163 and CD141.

In a preferred embodiment, the targeting unit comprises or consists of a moiety that binds to a surface molecule on human (h) APCs selected from the group consisting of hTGFβ receptor (including hTGFβR1, hTGFβR2, and hTGFβR3), hIL-10R, such as hIL-10RA and hIL-10RB, hIL-2R, hIL-4R, hIL-6R, hIL-11R, hIL-13R, hIL-27R, hIL-35R, hIL-37R, hGM-CSFR, hFLT3, hCCR7, hCD11b, hCD11c, hCD103, hCD14, hCD36, hCD205, hCD109, hVISTA, hMARCO, hMHCII, hCD83, hSIGLEC, hClec10A (hMGL), hASGR (hASGR1/hASGR2), hCD80, hCD86, hClec9A, hClec12A, hClec12B, hDCIR2, hLangerin, hMR, hDC-Sign, hTreml4, hDectin-1, hPDL1, hPDL2, hHVEM, hCD163 and hCD141.

The moiety may be a natural ligand, an antibody or part thereof, e.g. a scFv, or a synthetic ligand.

In one embodiment, the moiety is an antibody or part thereof, e.g. a scFv, with specificity for any of the aforementioned surface molecules, whose binding to the surface molecules results in the T cell epitopes comprised in the antigenic unit being presented in an anti-inflammatory, tolerogenic manner.

In another embodiment, the moiety is a synthetic ligand with specificity for any of the aforementioned surface molecules, whose binding to the surface molecules results in the T cell epitopes comprised in the antigenic unit being presented in an anti-inflammatory, tolerogenic manner. Protein modelling may be used to design such synthetic ligands.

In yet another embodiment, the moiety is a natural ligand. In one embodiment, the natural ligand is selected from the group consisting of TGFβ, IL-10, IL-2, IL-4, IL-6, IL-11, IL-13, IL-27, IL-35, IL-37, GM-CSF, FLT3L, CCL19, CCL21, ICAM-1 (Intercellular Adhesion Molecule 1 also known as CD54), keratin, VSIG-3, preferably the extracellular domain of VSIG-3, SCGB3A2, CTLA-4, preferably the extracellular domain of CTLA-4, PD-1, preferably the extracellular domain of PD-1 and BTLA, preferably the extracellular domain of BTLA.

In a preferred embodiment, the moiety is a human (h) natural ligand selected from the group consisting of hTGFβ, hIL-10, hIL-2, such as hIL-2 with SEQ ID NO: 55, hIL-4, hIL-6, hIL-11, hIL-13, hIL-27, hIL-35, hIL-37, hGM-CSF, such as hGM-CSF with SEQ ID NO: 56, hFLT3L, hCCL19, hCCL21, hICAM-1 (Intercellular Adhesion Molecule 1 also known as CD54), hkeratin, hVSIG-3, preferably the extracellular domain of hVSIG-3, hSCGB3A2, hCTLA-4, preferably the extracellular domain of hCTLA-4, such as the extracellular domain of hCTLA4 with SEQ ID NO: 51, hPD-1, preferably the extracellular domain of PD-1, such as the extracellular domain of hPD-1 with SEQ ID NO: 52 and hBTLA, preferably the extracellular domain of hBTLA.

In another embodiment, the targeting unit is or comprises IL10 or TGFβ, preferably human IL-10 or human TGFβ, including its isoforms TGFβ-1, TGFβ-2 and TGFβ-3.

In another embodiment, the targeting unit comprises or consists of an amino acid sequence having at least 80% sequence identity to that of human TGFβ. In one embodiment, the targeting unit comprises an amino acid sequence having at least 80% sequence identity to the amino acid sequence of SEQ ID NO: 54 or SEQ ID NO: 58 or SEQ ID NO: 59. In another embodiment, the targeting unit consists of an amino acid sequence having at least 80% sequence identity to the amino acid sequence of SEQ ID NO: 54 or SEQ ID NO: 58 or SEQ ID NO: 59.

In yet another embodiment, the targeting unit comprises an amino acid sequence having at least 85% sequence identity to the amino acid sequence of SEQ ID NO: 54 or SEQ ID NO: 58 or SEQ ID NO: 59, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the amino acid sequence of SEQ ID NO: 54 or SEQ ID NO: 58 or SEQ ID NO: 59.

In yet another embodiment, the targeting unit consists of an amino acid sequence having at least 85% sequence identity to the amino acid sequence of SEQ ID NO: 54 or SEQ ID NO: 58 or SEQ ID NO: 59, such as at least 86% or at least 87%, 88%, 89%, 90%, 91%, 92%, 93%, 94%, 95%, 96%, 97%, 98% or at least 99% sequence identity. In yet a further preferred embodiment, the targeting unit consists of the amino acid sequence of SEQ ID NO: 54 or SEQ ID NO: 58 or SEQ ID NO: 59.

In one preferred embodiment, the targeting unit comprises the amino acid sequence of SEQ ID NO: 54 or SEQ ID NO: 58 or SEQ ID NO: 59, except that at the most 80 amino acids have been substituted, deleted or inserted, such as at the most 75, 70, 65, 60, 55, 50, 45, 40, 35, 30, 25, 20, 15, 10, 9, 8, 7, 6, 5, 4, 3, 2 or 1 amino acid.

In one preferred embodiment, the targeting unit consists of the amino acid sequence of SEQ ID NO: 54 or SEQ ID NO: 58 or SEQ ID NO: 59, except that at the most 80 amino acids have been substituted, deleted or inserted, such as at the most 75, 70, 65, 60, 55, 50, 45, 40, 35, 30, 25, 20, 15, 10, 9, 8, 7, 6, 5, 4, 3, 2 or 1 amino acid.

In one preferred embodiment, the targeting unit comprises a nucleic acid sequence having at least 80% sequence identity to the nucleic acid sequence with SEQ ID NO: 60 or SEQ ID NO: 61 or SEQ ID NO: 62.

In a further preferred embodiment, the targeting unit comprises a nucleic acid sequence having at least 85% sequence identity to the nucleic acid sequence with SEQ ID NO: 60 or SEQ ID NO: 61 or SEQ ID NO: 62, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the nucleic acid sequence of SEQ ID NO: 60 or SEQ ID NO: 61 or SEQ ID NO: 62.

In a more preferred embodiment, the targeting unit consists of a nucleic acid sequence having at least 80% sequence identity to the nucleic acid sequence with SEQ ID NO: 60 or SEQ ID NO: 61 or SEQ ID NO: 62.

In a further preferred embodiment, the targeting unit consists of a nucleic acid sequence having at least 85% sequence identity to the nucleic acid sequence of SEQ ID NO: 60 or SEQ ID NO: 61 or SEQ ID NO: 62, such as at least 86% or at least 87%, 88%, 89%, 90%, 91%, 92%, 93%, 94%, 95%, 96%, 97%, 98% or at least 99% sequence identity. In yet another preferred embodiment, the targeting unit has the nucleic acid sequence of SEQ ID NO: 60 or SEQ ID NO: 61 or SEQ ID NO: 62.

In yet another embodiment, the targeting unit comprises or consists of an amino acid sequence having at least 80% sequence identity to that of human IL-10. In one embodiment, the targeting unit comprises an amino acid sequence having at least 80% sequence identity to the amino acid sequence of SEQ ID NO: 53. In another embodiment, the targeting unit consists of an amino acid sequence having at least 80% sequence identity to the amino acid sequence of SEQ ID NO: 53

In yet another embodiment, the targeting unit comprises an amino acid sequence having at least 85% sequence identity to the amino acid sequence of SEQ ID NO: 53, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the amino acid sequence of SEQ ID NO: 53.

In yet another embodiment, the targeting unit consists of an amino acid sequence having at least 85% sequence identity to the amino acid sequence of SEQ ID NO: 53, such as at least 86% or at least 87%, 88%, 89%, 90%, 91%, 92%, 93%, 94%, 95%, 96%, 97%, 98% or at least 99% sequence identity. In yet a further preferred embodiment, the targeting unit consists of the amino acid sequence of SEQ ID NO: 53.

In one preferred embodiment, the targeting unit comprises the amino acid sequence of SEQ ID NO: 53, except that at the most 35 amino acids have been substituted, deleted or inserted, such as at the most 30, 25, 20, 15, 14, 13, 12, 11, 10, 9, 8, 7, 6, 5, 4, 3, 2 or 1 amino acid.

In one preferred embodiment, the targeting unit consists of the amino acid sequence of SEQ ID NO: 53, except that at the most 35 amino acids have been substituted, deleted or inserted, such as at the most 30, 25, 20, 15, 14, 13, 12, 11, 10, 9, 8, 7, 6, 5, 4, 3, 2 or 1 amino acid.

In one preferred embodiment, the targeting unit comprises a nucleic acid sequence having at least 80% sequence identity to the nucleic acid sequence with SEQ ID NO: 63.

In a further preferred embodiment, the targeting unit comprises a nucleic acid sequence having at least 85% sequence identity to the nucleic acid sequence with SEQ ID NO: 63, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the nucleic acid sequence of SEQ ID NO: 63.

In a more preferred embodiment, the targeting unit consists of a nucleic acid sequence having at least 80% sequence identity to the nucleic acid sequence with SEQ ID NO: 63.

In a further preferred embodiment, the targeting unit consists of a nucleic acid sequence having at least 85% sequence identity to the nucleic acid sequence of SEQ ID NO: 63, such as at least 86% or at least 87%, 88%, 89%, 90%, 91%, 92%, 93%, 94%, 95%, 96%, 97%, 98% or at least 99% sequence identity. In yet another preferred embodiment, the targeting unit has the nucleic acid sequence of SEQ ID NO: 63.

In one embodiment, the targeting unit is or comprises SCGB3A2 or VSIG-3, preferably human VSIG-3, preferably the extracellular part, such as the extracellular domain, of human VSIG-3 or human SCGB3A2.

In another embodiment, the targeting unit comprises or consists of an amino acid sequence having at least 80% sequence identity to that of human SCGB3A2. In one embodiment, the targeting unit comprises an amino acid sequence having at least 80% sequence identity to the amino acid sequence of SEQ ID NO: 64. In another embodiment, the targeting unit consists of an amino acid sequence having at least 80% sequence identity to the amino acid sequence of SEQ ID NO: 64.

In yet another embodiment, the targeting unit comprises an amino acid sequence having at least 85% sequence identity to the amino acid sequence of SEQ ID NO: 64, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the amino acid sequence of SEQ ID NO: 64.

In yet another embodiment, the targeting unit consists of an amino acid sequence having at least 85% sequence identity to the amino acid sequence of SEQ ID NO: 64, such as at least 86% or at least 87%, 88%, 89%, 90%, 91%, 92%, 93%, 94%, 95%, 96%, 97%, 98% or at least 99% sequence identity. In yet a further preferred embodiment, the targeting unit consists of the amino acid sequence of SEQ ID NO: 64.

In one preferred embodiment, the targeting unit comprises the amino acid sequence of SEQ ID NO: 64, except that at the most 18 amino acids have been substituted, deleted or inserted, such as at the most 17, 16, 15, 14, 13, 12, 11, 10, 9, 8, 7, 6, 5, 4, 3, 2 or 1 amino acid.

In one preferred embodiment, the targeting unit consists of the amino acid sequence of SEQ ID NO: 64, except that at the most 18 amino acids have been substituted, deleted or inserted, such as at the most 17, 16, 15, 14, 13, 12, 11, 10, 9, 8, 7, 6, 5, 4, 3, 2 or 1 amino acid.

In one preferred embodiment, the targeting unit comprises a nucleic acid sequence having at least 80% sequence identity to the nucleic acid sequence with SEQ ID NO: 65.

In a further preferred embodiment, the targeting unit comprises a nucleic acid sequence having at least 85% sequence identity to the nucleic acid sequence with SEQ ID NO: 65, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the nucleic acid sequence of SEQ ID NO: 65.

In a more preferred embodiment, the targeting unit consists of a nucleic acid sequence having at least 80% sequence identity to the nucleic acid sequence with SEQ ID NO: 65.

In a further preferred embodiment, the targeting unit consists of a nucleic acid sequence having at least 85% sequence identity to the nucleic acid sequence of SEQ ID NO: 65, such as at least 86% or at least 87%, 88%, 89%, 90%, 91%, 92%, 93%, 94%, 95%, 96%, 97%, 98% or at least 99% sequence identity. In yet another preferred embodiment, the targeting unit has the nucleic acid sequence of SEQ ID NO: 65.

In yet another embodiment, the targeting unit comprises or consists of an amino acid sequence having at least 80% sequence identity to that of the extracellular domain of human VSIG-3. In one embodiment, the targeting unit comprises an amino acid sequence having at least 80% sequence identity to the amino acid sequence of SEQ ID NO: 66. In another embodiment, the targeting unit consists of an amino acid sequence having at least 80% sequence identity to the amino acid sequence of SEQ ID NO: 66.

In yet another embodiment, the targeting unit comprises an amino acid sequence having at least 85% sequence identity to the amino acid sequence of SEQ ID NO: 66, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the amino acid sequence of SEQ ID NO: 67.

In yet another embodiment, the targeting unit consists of an amino acid sequence having at least 85% sequence identity to the amino acid sequence of SEQ ID NO: 66, such as at least 86% or at least 87%, 88%, 89%, 90%, 91%, 92%, 93%, 94%, 95%, 96%, 97%, 98% or at least 99% sequence identity. In yet a further preferred embodiment, the targeting unit consists of the amino acid sequence of SEQ ID NO: 66.

In one preferred embodiment, the targeting unit comprises the amino acid sequence of SEQ ID NO: 66, except that at the most 86 amino acids have been substituted, deleted or inserted, such as at the most 85, 80, 75, 70, 65, 60, 55, 50, 45, 40, 35, 30, 25, 20, 15, 10, 9, 8, 7, 6, 5, 4, 3, 2 or 1 amino acid.

In one preferred embodiment, the targeting unit consists of the amino acid sequence of SEQ ID NO: 66, except that at the most 86 amino acids have been substituted, deleted or inserted, such as at the most 85, 80, 75, 70, 65, 60, 55, 50, 45, 40, 35, 30, 25, 20, 15, 10, 9, 8, 7, 6, 5, 4, 3, 2 or 1 amino acid.

In one preferred embodiment, the targeting unit comprises a nucleic acid sequence having at least 80% sequence identity to the nucleic acid sequence with SEQ ID NO: 67.

In a further preferred embodiment, the targeting unit comprises a nucleic acid sequence having at least 85% sequence identity to the nucleic acid sequence with SEQ ID NO: 67, such as at least 86% or at least 87%, 88%, 89%, 90%, 91%, 92%, 93%, 94%, 95%, 96%, 97%, 98% or at least 99% sequence identity. In yet a further preferred embodiment, the targeting unit comprises the nucleic acid sequence of SEQ ID NO: 67.

In a more preferred embodiment, the targeting unit consists of a nucleic acid sequence having at least 80% sequence identity to the nucleic acid sequence with SEQ ID NO: 67.

In a further preferred embodiment, the targeting unit consists of a nucleic acid sequence having at least 85% sequence identity to the nucleic acid sequence of SEQ ID NO: 67, such as at least 86% or at least 87%, 88%, 89%, 90%, 91%, 92%, 93%, 94%, 95%, 96%, 97%, 98% or at least 99% sequence identity. In yet another preferred embodiment, the targeting unit has the nucleic acid sequence of SEQ ID NO: 67. In yet another embodiment, the targeting unit is or comprises an antibody or part thereof, e.g. a scFv, with specificity for CD205, such as anti-human CD205.

### Multimerization Unit/Dimerization Unit

The first polypeptide encoded by the first nucleic acid sequence comprised in the vector of the invention comprises a multimerization unit, such as a dimerization unit.

The term “multimerization unit” as used herein refers to a sequence of nucleotides or amino acids between the antigenic unit and the targeting unit. In addition to connecting the antigenic unit and the targeting unit, the multimerization unit facilitates multimerization of/joins multiple polypeptides, such as two, three, four or more polypeptides, into a multimeric protein, such as a dimeric protein, a trimeric protein or a tetrameric protein. Furthermore, the multimerization unit also provides the flexibility in the multimeric protein to allow optimal binding of the targeting unit to the surface molecules on the APCs, even if they are located at variable distances. The multimerization unit may be any unit that fulfils one or more of these requirements.

Multimerization Unit that Facilitates Multimerization of/Joins More than Two Polypeptides

In one embodiment, the multimerization unit is a trimerization unit, such as a collagen-derived trimerization unit, such as a human collagen-derived trimerization domain, such as human collagen derived XVIII trimerization domain (see for instance A. Alvarez-Cienfuegos et al., Sci Rep 6, 28643 (2016)) or human collagen XV trimerization domain. Thus, in one embodiment, the multimerization unit is a trimerization unit that comprises or consists of the nucleic acid sequence with SEQ ID NO: 116, or an amino acid sequence encoded by said nucleic acid sequence. In another embodiment, the trimerization unit is the C-terminal domain of T4 fibritin. Thus, in one embodiment, the multimerization unit is a trimerization unit that comprises or consists of the amino acid sequence with SEQ ID NO: 117.

In another embodiment, the multimerization unit is a tetramerization unit, such as a domain derived from p53, optionally further comprising a hinge region as described below. Thus, in one embodiment, the multimerization unit is a tetramerization unit that comprises or consists of the nucleic acid sequence with SEQ ID NO: 113, or an amino acid sequence encoded by said nucleic acid sequence, optionally further comprising a hinge region as described below.

### Dimerization Unit

The term “dimerization unit” as used herein, refers to a sequence of nucleotides or amino acids between the antigenic unit and the targeting unit. In addition to connect the antigenic unit and the targeting unit, the dimerization unit facilitates dimerization of/joins two monomeric polypeptides into a dimeric protein. Furthermore, the dimerization unit also provides the flexibility in the dimeric protein to allow optimal binding of the targeting unit to the surface molecules on the APCs, even if they are located at variable distances. The dimerization unit may be any unit that fulfils these requirements.

Accordingly, in one embodiment the first polypeptide comprises a dimerization unit comprising a hinge region. In another embodiment, the dimerization unit comprises a hinge region and another domain that facilitates dimerization. In yet another embodiment, the dimerization unit comprises a hinge region, a dimerization unit linker and another domain that facilitates dimerization, wherein the dimerization unit linker connects the hinge region and the other domain that facilitates dimerization. In one embodiment, the dimerization unit linker is a glycine-serine rich linker, preferably GGGSSGGGSG (SEQ ID NO: 118), i.e. the dimerization unit comprises a glycine-serine rich dimerization unit linker and preferably the dimerization unit linker GGGSSGGGSG (SEQ ID NO: 118).

The term “hinge region” refers to an amino acid sequence comprised in the dimerization unit that contributes to joining two of the polypeptides, i.e. facilitates the formation of a dimeric protein. In the context of a multimerization unit that facilitates multimerization of/joins more than two polypeptides, the term “hinge region” refers to an amino acid sequence comprised in such multimerization unit that contributes to joining more than two polypeptides, e.g. three or four polypeptides and/or functioning as a flexible spacer, allowing the two targeting units of the multimeric protein to bind simultaneously to multiple surface molecules on APCs, even if they are located at variable distances. The hinge region may be Ig derived, such as derived from IgG, e.g. IgG1, IgG2 or IgG3. In one embodiment, the hinge region is derived from IgM, e.g. comprising or consisting of the nucleotide sequence with SEQ ID NO: 119 or an amino acid sequence encoded by said nucleic acid sequence.

The hinge region may contribute to the dimerization through the formation of covalent bond(s), e.g. disulfide bridge(s) between cysteines. Thus, in one embodiment, the hinge region has the ability to form one or more covalent bonds. Preferably, the covalent bond is a disulfide bridge.

In one embodiment, the dimerization unit comprises or consists of a hinge exon h1 and hinge exon h4 (human hinge region 1 and human hinge region 4) of IgG3, preferably having an amino acid sequence of at least 80% sequence identity to the amino acid sequence 1-27 of SEQ ID NO: 1.

In a preferred embodiment, the dimerization unit comprises or consists of a hinge exon h1 and hinge exon h4 with an amino acid sequence of at least 85% sequence identity to the amino acid sequence 1-27 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a preferred embodiment, the dimerization unit comprises or consists of a hinge exon h1 and hinge exon h4 with the amino acid sequence 1-27 of SEQ ID NO: 1.

In one preferred embodiment, the dimerization unit comprises or consists of the amino acid sequence 1-27 of SEQ ID NO: 1, except that at the most four amino acids have been substituted, deleted or inserted, such as at the most three amino acids, such as at the most two amino acids or such as at the most one amino acid.

In one preferred embodiment, the dimerization unit comprises or consists of a nucleic acid sequence having at least 80% sequence identity to the nucleic acid sequence with SEQ ID NO: 10.

In a further preferred embodiment, the dimerization unit comprises or consists of a nucleic acid sequence having at least 85% sequence identity to the nucleic acid sequence with SEQ ID NO: 10, such as at least 86% or at least 87%, 88%, 89%, 90%, 91%, 92%, 93%, 94%, 95%, 96%, 97%, 98% or at least 99% sequence identity.

In yet a further preferred embodiment, the dimerization unit comprises or consists of the nucleic acid sequence of SEQ ID NO: 10.

In another embodiment, the dimerization unit comprises another domain that facilitates dimerization, said other domain is an immunoglobulin domain, such as an immunoglobulin constant domain (C domain), such as a CH1 domain, a CH2 domain or a carboxyterminal C domain (i.e. a CH3 domain), or a sequence that is substantially identical to such C domains or a variant thereof. Preferably, the other domain that facilitates dimerization is a carboxyterminal C domain derived from IgG. More preferably, the other domain that facilitates dimerization is a carboxyterminal C domain derived from IgG3.

In one embodiment, the dimerization unit comprises or consists of a carboxyterminal C domain derived from IgG3 with an amino acid sequence having at least 80% sequence identity to the amino acid sequence 38-144 of SEQ ID NO: 1.

In a preferred embodiment, the dimerization unit comprises or consists of a carboxyterminal C domain derived from IgG3 with an amino acid sequence having at least 85% sequence identity to the amino acid sequence 38-144 of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In a preferred embodiment, the dimerization unit comprises a carboxyterminal C domain derived from IgG3 with the amino acid sequence 38-144 of SEQ ID NO: 1.

In one preferred embodiment, the dimerization unit comprises or consists of the amino acid sequence 38-144 of SEQ ID NO: 1, except that at the most 16 amino acids have been substituted, deleted or inserted, such as at the most 15, 14, 13, 12, 11, 10, 9, 8, 7, 6, 5, 4, 3, 2, or 1 amino acid.

In one preferred embodiment, the dimerization unit comprises or consists of a nucleic acid sequence having at least 80% sequence identity to the nucleic acid sequence with SEQ ID NO: 11.

In a further preferred embodiment, the dimerization unit comprises or consists of a nucleic acid sequence having at least 85% sequence identity to the nucleic acid sequence with SEQ ID NO: 11, such as at least 86% or at least 87%, 88%, 89%, 90%, 91%, 92%, 93%, 94%, 95%, 96%, 97%, 98% or at least 99% sequence identity. In yet a further preferred embodiment, the dimerization unit comprises or consists of the nucleic acid sequence of SEQ ID NO: 11.

The immunoglobulin domain contributes to dimerization through non-covalent interactions, e.g. hydrophobic interactions. Thus, in one embodiment, the immunoglobulin domain has the ability to form dimers via noncovalent interactions. Preferably, the noncovalent interactions are hydrophobic interactions.

It is preferred that if the dimerization unit comprises a CH3 domain, it does not comprise a CH2 domain and vice versa.

In a preferred embodiment, the dimerization unit comprises a hinge exon h1, a hinge exon h4, a dimerization unit linker and a CH3 domain of human IgG3. In a further preferred embodiment, the dimerization unit comprises a polypeptide consisting of hinge exon h1, hinge exon h4, a dimerization unit linker and a CH3 domain of human IgG3. In another preferred embodiment, the dimerization unit consists of a polypeptide consisting of hinge exon h1, hinge exon h4, a dimerization unit linker and a CH3 domain of human IgG3.

In one embodiment, the dimerization unit comprises an amino acid sequence having at least 80% sequence identity to the amino acid sequence of SEQ ID NO: 1.

In a preferred embodiment, the dimerization unit comprises an amino acid sequence having at least 85% sequence identity to the amino acid sequence of SEQ ID NO: 1, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In an even more preferred embodiment, the dimerization unit comprises the amino acid sequence of SEQ ID NO: 1.

In a more preferred embodiment the dimerization unit consists of an amino acid sequence having at least 80% sequence identity to the amino acid sequence of SEQ ID NO: 1, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In an even more preferred embodiment, the dimerization unit consists of the amino acid sequence of SEQ ID NO: 1.

In one preferred embodiment, the dimerization unit comprises or consists of the amino acid sequence of SEQ ID NO: 1, except that at the most 28 amino acids have been substituted, deleted or inserted, such as at the most 25, 20, 15, 14, 13, 12, 11, 10, 9, 8, 7, 6, 5, 4, 3, 2 or 1 amino acids.

In one preferred embodiment, the dimerization unit comprises or consists of a nucleic acid sequence having at least 80% sequence identity to the nucleic acid sequence with SEQ ID NO: 12.

In a further preferred embodiment, the dimerization unit comprises or consists of a nucleic acid sequence having at least 85% sequence identity to the nucleic acid sequence with SEQ ID NO: 12, such as at least 86% or at least 87%, 88%, 89%, 90%, 91%, 92%, 93%, 94%, 95%, 96%, 97%, 98% or at least 99% sequence identity.

In yet a further preferred embodiment, the dimerization unit comprises or consists of the nucleic acid sequence of SEQ ID NO: 12.

In the first polypeptide encoded by the first nucleic acid sequence, the multimerization unit, e.g. dimerization unit, may have any orientation with respect to antigenic unit and targeting unit. In one embodiment, the antigenic unit is connected to the C-terminal end of the multimerization/dimerization unit (e.g. via a unit linker) with the targeting unit being connected to the N-terminal end of the multimerization/dimerization unit. In another embodiment, the antigenic unit is connected to the N-terminal end of the multimerization/dimerization unit (e.g. via a unit linker) with the targeting unit being connected to the C-terminal end of the multimerization/dimerization unit. It is preferred that the antigenic unit is connected to the C-terminal end of the multimerization/dimerization unit, e.g. via a linker, preferably via the unit linker, and the targeting unit is connected to the N-terminal end of the multimerization/dimerization unit.

### Antigenic Unit

The first polypeptide encoded by the first nucleic acid sequence comprised in the vector of the invention comprises an antigenic unit comprising one or more T-cell epitopes of a self-antigen such as one or more epitopes of regulatory T cells (Tregs) or one or more inhibitory neoantigens, an allergen, an alloantigen or a xenoantigen.

T cell epitopes suitable for inclusion into the antigenic unit may be known in the art, i.e. have been studied, proposed and/or verified to be involved and of relevance for a certain immune disease and published, e.g. in the scientific literature.

In one embodiment, the antigenic unit comprises one or more T cell epitopes of a self-antigen, i.e. one T cell epitope of a self-antigen or more than one T cell epitope of a self-antigen, i.e. multiple T cell epitopes of a self-antigen. In one embodiment, the multiple T cell epitopes are of the same self-antigen, i.e. comprised in the same self-antigen. In another embodiment, the multiple T cell epitopes are of multiple different self-antigens, i.e. comprised in different self-antigens.

The terms “multiple”, “a plurality” and “several” are used herein interchangeably with “more than one”.

By way of example, myelin basic protein (MBP), proteolipid protein (PLP), myelin-associated glycoprotein (MAG), myelin oligodendrocyte glycoprotein (MOG) and myelin-associated basic oligodendrocytic protein (MOBP) have all been studied and proposed as self-antigens involved in multiple sclerosis (MS) and the antigenic unit may comprise e.g. one or more T cell epitopes of MBP, i.e. one T cell epitope of MBP or multiple T cell epitopes of MBP. Further, the antigenic unit may comprise multiple T cell epitopes of e.g. MOG and PLP, e.g. one or multiple T cell epitopes of MOG and one or multiple T cell epitopes of PLP.

In another embodiment, the antigenic unit comprises one or more T cell epitopes of an allergen, i.e. one T cell epitope of an allergen or more than one T cell epitope of an allergen, i.e. multiple T cell epitopes of an allergen. In one embodiment, the multiple T cell epitopes are of the same allergen, i.e. comprised in the same allergen. In another embodiment, the multiple T cell epitopes are of multiple different allergens, i.e. comprised in different allergens.

By way of example, Fel d 1, Fel d 4 and Fel d 7 are three of the most prominent cat allergens, accounting for the majority of human cat allergies and the antigenic unit may comprise e.g. one or more T cell epitopes of Fel d 1, i.e. one T cell epitope of Fel d 1 or multiple T cell epitopes of Fel d 1. Further, the antigenic unit may comprise multiple T cell epitopes of e.g. Fel d 4 and Fel d 7, e.g. one or multiple T cell epitopes of Fel d 4 and one or multiple T cell epitopes of Fel d 7.

In another embodiment, the antigenic unit comprises one or more T cell epitopes of an alloantigen/xenoantigen, i.e. one T cell epitope of an alloantigen/xenoantigen or more than one T cell epitope of an alloantigen/xenoantigen, i.e. multiple T cell epitopes of an alloantigen/xenoantigen. In one embodiment, the multiple T cell epitopes are of the same alloantigen/xenoantigen, i.e. comprised in the same alloantigen/xenoantigen. In another embodiment, the multiple T cell epitopes are of multiple different alloantigen/xenoantigens, i.e. comprised in different alloantigens/xenoantigens.

In one embodiment, the antigenic unit includes one T cell epitope. In another embodiment, the antigenic unit includes more than one T cell epitope, i.e. multiple T cell epitopes.

In one embodiment, the vectors of the invention/the construct encoded by such vectors are for use in an individualized treatment, i.e. designed specifically for a particular subject/one patient. In another embodiment, the vectors of the invention/construct encoded by such vectors are for general use in a patient population or patients, i.e. an off-the-shelf treatment.

### Individualized Constructs

For individualized constructs, T cell epitopes are selected for inclusion into the antigenic unit which are optimized for the patient who will receive treatment with the vector encoding such construct. This will increase the therapeutic effect compared to an off-the-shelf treatment comprising the construct.

The antigenic unit of an individualized construct may be designed as follows, as exemplified for a patient suffering from MS:
1) The patient's HLA class I and/or HLA class II alleles are determined
2) T cell epitopes are identified comprised in one or more self-antigens (e.g. self-antigens which have been studied, proposed and/or verified as self-antigens involved in MS)
3) T cell epitopes are selected based on predicted binding to the patient's HLA class I and/or class II alleles
4) One or more test constructs are designed and produced, and the T cell epitopes are optionally arranged in the antigenic unit of the constructs as described in this application.

The T cell epitopes are selected in the method described above based on their predicted ability to bind to the patient's HLA class I/II alleles, i.e. selected in silico using predictive HLA-binding algorithms. After having identified relevant epitopes, the epitopes are ranked according to their ability to bind to the patient's HLA class I/II alleles and the epitopes that are predicted to bind best are selected to be included in the antigenic unit of the test constructs.

Any suitable HLA-binding algorithm may be used, such as one of the following: Available software analysis of peptide-MHC binding (IEDB, NetMHCpan and NetMHCIIpan) may be downloaded or used online from the following websites: www.iedb.org/
services.healthtech.dtu.dk/service.php?NetMHCpan-4.0
services.healthtech.dtu.dk/service.php?NetMHCIIpan-3.2

### Off-the-Shelf Tolerance Inducing Constructs

The antigenic unit of an off-the-shelf construct encoded by a vector of the invention may include discrete T cell epitopes, hotspots of minimal T cell epitopes or both. Such antigenic unit preferably includes hotspots of minimal T cell epitopes, i.e. one or more regions of an antigen that contain multiple minimal T cell epitopes (e.g. having a length of from 7-15 amino acids) that are predicted to be presented by different HLA alleles to cover a broad range of subjects, e.g. an ethnic population or even a world population. By including such hotspots, chances are maximized that the construct will induce tolerance in a broad range of subjects.

### Further Embodiments of the Antigenic Unit

The T cell epitope comprised in the antigenic unit of the first polypeptide encoded by vector of the invention has a length of from 7 to about 200 amino acids, with the longer T cell epitopes possibly including hotspots of minimal T cell epitopes.

In one embodiment, the antigenic unit comprises one or more T cell epitopes with a length of from 7 to 150 amino acids, preferably of from 7 to 100 amino acids, e.g. from 9 to 100 amino acids or from 15 to 100 amino acids or from 9 to 60 amino acids or from 9 to 30 amino acids or from 15 to 60 of from 15 to 30 or from 20 to 75 amino acids or from 25 to 50 amino acids.

T cell epitopes having a length of about 60 to 200 amino acids may be split into shorter sequences and included into the antigenic unit separated by linkers, e.g. the linkers which are described herein. By way of example, a T cell epitope having a length of 150 amino acids may be split into 3 sequences of 50 amino acids each, and included into the antigenic unit, with linkers separating the 3 sequences from each other.

In one embodiment, the length of one T cell epitope is adjusted such that the protein comprising the T cell epitope does not fold correctly. For example, Fel d 1, the most prominent cat allergen, is a protein formed by two heterodimers, with each dimer being composed of two chains, chain 1 comprising 70 amino acid residues and chain 2, comprising 90 or 92 residues. Including long T cell epitopes of both chains into the antigenic unit may result in the proteins folding correctly and, if more than one IgE on the subject's mast cells and basophiles bind to the antigenic unit of the construct comprising T cell epitopes, might elicit an allergic reaction.

Thus, if a longer T cell epitope is included in the antigenic unit, protein folding may be tested in vitro by e.g. ELISA, using an antibody against the protein (e.g. cat allergen) and determining whether the antibody binds to the T cell epitope. If the antibody binds to the T cell epitope, its length may be adjusted or it may be split into shorter sequences, as described herein.

In one embodiment, the T cell epitope has a length suitable for presentation by MHC (major histocompatibility complex). There are two primary classes of MHC molecules, MHC class I and MHC II. The terms MHC class I and MHC class II are interchangeably used herein with HLA class I and HLA class II. HLA (human leukocyte antigen) is a major histocompatibility complex in humans. Thus, in a preferred embodiment, the antigenic unit comprises T cell epitopes having a length suitable for specific presentation on MHC class I or MHC class II. In one embodiment, the T cell epitope has a length of from 7 to 11 amino acids for MHC class I presentation. In another embodiment, the T cell epitope has a length of about 15 amino acids for MHC class II presentation.

### Arrangement and Details of T Cell Epitopes

The number of T cell epitopes in the antigenic unit may vary, and depends on the length and number of other elements included in the antigenic unit, e.g. T cell epitope linkers as described in this application.

In one embodiment, the antigenic unit comprises up to 3500 amino acids, such as from 60 to 3500 amino acids, e.g. from about 80 or about 100 or about 150 amino acids to about 3000 amino acids, such as from about 200 to about 2500 amino acids, such as from about 300 to about 2000 amino acids or from about 400 to about 1500 amino acids or from about 500 to about 1000 amino acids.

In one embodiment, the antigenic unit comprises 1 to 10 T cell epitopes such as 1, 2, 3, 4, 5, 6, 7, 8 or 9 or 10 T cell epitopes or 11 to 20 T cell epitopes, such as 11, 12, 13, 14, 15, 16, 17, 18, 19 or 20 T cell epitopes or 21 to 30 T cell epitopes, such as 21, 22, 23, 24, 25, 26, 27, 28, 29 or 30 T cell epitopes or 31 to 40 T cell epitopes, such as 31, 32, 33, 34, 35, 36, 37, 38, 39 or 40 T cell epitopes or 41 to 50 T cell epitopes, such as 41, 42, 43, 44, 45, 46, 47, 48, 49 or 50 T cell epitopes.

In one embodiment, the T cell epitopes are randomly arranged in the antigenic unit. In another embodiment, one or more of the following methods for arranging them in the antigenic unit may be used.

In one embodiment, the T cell epitopes are arranged in the order of more antigenic to less antigenic in the direction from multimerization unit (such as a dimerization unit) to the end of the antigenic unit (see FIG. 5). Alternatively, particularly if the hydrophilicity/hydrophobicity varies greatly among the T cell epitopes, the most hydrophobic T cell epitope(s) may be positioned substantially in the middle of the antigenic unit and the most hydrophilic T cell epitope(s) is/are positioned closest to the multimerization unit or the end of the antigenic unit.

Since a true positioning in the middle of the antigenic unit is only possible if the antigenic unit comprises an odd number of T cell epitopes, the term “substantially” in this context refers to antigenic units comprising an even number of T cell epitopes, wherein the most hydrophobic T cell epitopes are positioned as close to the middle as possible.

By way of example, an antigenic unit comprises 5 T cell epitopes, which are arranged as follows: 1-2-3*-4-5; with 1, 2, 3*, 4 and 5 each being a different T cell epitope and—being a T cell epitope linker and * indicates the most hydrophobic T cell epitope, which is positioned in the middle of the antigenic unit.

In another example, an antigenic unit comprises 6 T cell epitopes, which are arranged as follows: 1-2-3*-4-5-6 or, alternatively, as follows: 1-2-4-3*-5-6; with 1, 2, 3*, 4, 5 and 6 each being a T cell epitope and—being a T cell epitope linker and * indicates the most hydrophobic T cell epitope, which is positioned substantially in the middle of the antigenic unit.

Alternatively, the T cell epitopes may be arranged alternating between a hydrophilic and a hydrophobic T cell epitope. Optionally, GC rich sequences encoding T cell epitopes are arranged in such a way, that GC clusters are avoided. In one embodiment, GC rich sequences encoding T cell epitopes are arranged such that there is at least one non-GC rich sequence between them.

### T Cell Epitope Linkers

If the antigenic unit comprises multiple T cell epitopes, the epitopes are preferably separated by T cell epitope linkers. This ensures that each T cell epitope is presented in an optimal way to the immune system. If the antigenic unit comprises n T cell epitopes, it preferably comprises n−1 T cell epitope linkers, separating each T cell epitope from one or two other T cell epitopes.

In one embodiment, the T cell epitope linkers are designed to be non-immunogenic. A T cell epitope linker may be a rigid linker, meaning that that it does not allow the two amino acid sequences that it connects to substantially move freely relative to each other. Alternatively, it may be a flexible linker, i.e. a linker that allows the two amino acid sequences that it connects to substantially move freely relative to each other. Both types of linkers are useful. In one embodiment, the linker is a flexible linker, which allows for presenting the T cell epitope in an optimal manner to the immune system, even if the antigenic unit comprises a large number of T cell epitopes.

In one embodiment, the T cell epitope linker is a peptide consisting of from 4 to 40 amino acids, e.g. 35, 30, 25 or 20 amino acids, such as from 4 to 20 amino acids, e.g. from 5 to 20 amino acids or 5 to 15 amino acids or 8 to 20 amino acids or 8 to 15 amino acids 10 to 15 amino acids or 8 to 12 amino acids. In one embodiment, the T cell epitope linker consists of 10 amino acids.

In one embodiment, all T cell epitope linkers comprised in the antigenic unit are identical. If, however, one or more of the T cell epitopes comprise a sequence similar to that of the linker, it may be an advantage to substitute the neighboring T cell epitope linker(s) with a linker of a different sequence. Also, if a T cell epitope/linker junction is predicted to constitute an epitope in itself, then it is preferred to use a T cell epitope linker of a different sequence.

In one embodiment, the T cell epitope linker is a flexible linker, preferably a flexible linker which comprises small, non-polar (e.g. glycine, alanine or leucine) or polar (e.g. serine or threonine) amino acids. The small size of these amino acids provides flexibility and allows for mobility of the connected amino acid sequences. The incorporation of serine or threonine can maintain the stability of the linker in aqueous solutions by forming hydrogen bonds with the water molecules, and therefore reduces the unfavorable interaction between the linker and antigens. In one embodiment, the flexible linker is a serine (S) and/or glycine (G) rich linker, i.e. a linker comprising several serine and/or several glycine residues. Preferred examples are GGGGSGGGSS (SEQ ID NO: 75), GGGSG (SEQ ID NO: 76), GGSGG (SEQ ID NO: 77), SGSSGS (SEQ ID NO: 78) or multiple variants thereof such as GGGGSGGGGS (SEQ ID NO: 79), (GGGGS)m (SEQ ID NO: 80), (GGGSS)m (SEQ ID NO: 81), (GGSGG)m (SEQ ID NO: 82), (GGGSG)m (SEQ ID NO: 83) or (SGSSGS)m (SEQ ID NO: 84), where m is an integer from 1 to 5, e.g., 1, 2, 3, 4, or 5, In a preferred embodiment, m is 2. In another preferred embodiment, the serine and/or glycine rich linker further comprises at least one leucine (L) residue, such as at least 1 or at least 2 or at least 3 leucine residues, e.g. 1, 2, 3 or 4 leucine residues.

In one embodiment, the T cell epitope linker comprises or consists of LGGGS (SEQ ID NO: 85), GLGGS (SEQ ID NO: 86), GGLGS (SEQ ID NO: 87), GGGLS (SEQ ID NO: 88) or GGGGL (SEQ ID NO: 89). In another embodiment, the T cell epitope linker comprises or consists of LGGSG (SEQ ID NO: 90), GLGSG (SEQ ID NO: 91), GGLSG (SEQ ID NO: 92), GGGLG (SEQ ID NO: 93) or GGGSL (SEQ ID NO: 94). In yet another embodiment, the T cell epitope linker comprises or consists of LGGSS (SEQ ID NO: 95), GLGSS (SEQ ID NO: 96) or GGLSS (SEQ ID NO: 97).

In yet another embodiment, the T cell epitope linker comprises or consists of LGLGS (SEQ ID NO: 98), GLGLS (SEQ ID NO: 99), GLLGS (SEQ ID NO: 100), LGGLS (SEQ ID NO: 101) or GLGGL (SEQ ID NO: 102). In yet another embodiment, the T cell epitope linker comprises or consists of LGLSG (SEQ ID NO: 103), GLLSG (SEQ ID NO: 104), GGLSL (SEQ ID NO: 105), GGLLG (SEQ ID NO: 106) or GLGSL (SEQ ID NO: 107). In yet another embodiment, the T cell epitope linker comprises or consists of LGLSS (SEQ ID NO: 108) or GGLLS (SEQ ID NO: 109).

In another embodiment, the T cell epitope linker is serine-glycine linker that has a length of 10 amino acids and comprises 1 or 2 leucine residues.

In one embodiment, the T cell epitope linker comprises or consists of LGGGSGGGGS (SEQ ID NO: 110), GLGGSGGGGS (SEQ ID NO: 111), GGLGSGGGGS (SEQ ID NO: 112), GGGLSGGGGS (SEQ ID NO: 155) or GGGGLGGGGS (SEQ ID NO: 114). In another embodiment, the T cell epitope linker comprises or consists of LGGSGGGGSG (SEQ ID NO: 115), GLGSGGGGSG (SEQ ID NO: 156), GGLSGGGGSG (SEQ ID NO: 157), GGGLGGGGSG (SEQ ID NO: 158) or GGGSLGGGSG (SEQ ID NO: 159). In yet another embodiment, the T cell epitope linker comprises or consists of LGGSSGGGSS (SEQ ID NO: 121), GLGSSGGGSS (SEQ ID NO: 122), GGLSSGGGSS (SEQ ID NO: 123), GGGLSGGGSS (SEQ ID NO: 124) or GGGSLGGGSS (SEQ ID NO: 125).

In a further embodiment, the T cell epitope linker comprises or consists of LGGGSLGGGS (SEQ ID NO: 126), GLGGSGLGGS (SEQ ID NO: 127), GGLGSGGLGS (SEQ ID NO: 128), GGGLSGGGLS (SEQ ID NO: 129) or GGGGLGGGGL (SEQ ID NO: 130). In another embodiment, the T cell epitope linker comprises or consists of LGGSGLGGSG (SEQ ID NO: 131), GLGSGGLGSG (SEQ ID NO: 132), GGLSGGGLSG (SEQ ID NO: 133), GGGLGGGGLG (SEQ ID NO: 134) or GGGSLGGGSL (SEQ ID NO: 135). In yet another embodiment, the T cell epitope linker comprises or consists of LGGSSLGGSS (SEQ ID NO: 136), GLGSSGLGSS (SEQ ID NO: 137) or GGLSSGGLSS (SEQ ID NO: 138).

In yet another embodiment, the subunit linker comprises or consists of GSGGGA (SEQ ID NO: 139), GSGGGAGSGGGA (SEQ ID NO: 140), GSGGGAGSGGGAGSGGGA (SEQ ID NO: 141), GSGGGAGSGGGAGSGGGAGSGGGA (SEQ ID NO: 142) or GENLYFQSGG (SEQ ID NO: 143). In yet another embodiment, the subunit linker comprises or consists of SGGGSSGGGS (SEQ ID NO: 144), SSGGGSSGGG (SEQ ID NO: 145), GGSGGGGSGG (SEQ ID NO: 146), GSGSGSGSGS (SEQ ID NO: 147), GGGSSGGGSG (SEQ ID NO: 118), GGGSSS (SEQ ID NO: 149), GGGSSGGGSSGGGSS (SEQ ID NO: 150) or GLGGLAAA (SEQ ID NO: 151).

In another embodiment, the T cell epitope linker is a rigid linker. Such rigid linkers may be useful to efficiently separate (larger) T cell epitopes and prevent their interferences with each other. In one embodiment, the T cell epitope linker comprises or consists of KPEPKPAPAPKP (SEQ ID NO: 152), AEAAAKEAAAKA (SEQ ID NO: 153), (EAAAK)m (SEQ ID NO: 154), PSRLEEELRRRLTEP (SEQ ID NO: 160) or SACYCELS (SEQ ID NO: 161).

In yet another embodiment, the T cell epitope linker comprises or consists of the sequence TQKSLSLSPGKGLGGL (SEQ ID NO: 162). In yet another embodiment, the T cell epitope linker comprises or consists of the sequence SLSLSPGKGLGGL (SEQ ID NO: 163).

In yet another embodiment, the T cell epitope linker comprises or consists of GGSAGGSGSGSSGGSSGASGTGTAGGTGSGSGTGSG (SEQ ID NO: 164) or GGSGGGSEGGGSEGGGSEGGGSEGGGSEGGGSGGGS (SEQ ID NO: 165) or EPKSCDTPPPCPRCP (SEQ ID NO: 166).

In yet another embodiment, the T cell epitope linker is a cleavable linker, e.g. a linker which includes one or more recognition sites for endopeptidases, e.g. endopeptidases such as furin, caspases, cathepsins and the like.

Examples of T cell epitope linkers are disclosed in paragraphs [0098]-[0099] and in the recited sequences of WO 2020/176797A1, in paragraphs [0135] to [0139] of US 2019/0022202A1, in WO 2017/118695A1 and in WO 2021/219897A1, all of which are incorporated herein by reference.

### Allergens

The vectors encoding a construct as described herein are useful for inducing tolerance to a range of different protein allergens, e.g. T cell epitopes of allergens that can be encoded by a nucleic acid sequence comprised in the first nucleic acid sequence of the vector of the invention, including protein allergens that undergo post-translational modifications.

The one or more T cell epitopes comprised in the antigenic unit may be derived from the following allergens:

In some embodiments, the allergen is a food allergen. In some embodiments, the allergen is a shellfish allergen. In some embodiments, the allergen is tropomyosin, in other embodiments the allergen is arginine kinase, myosin light chain, sarcoplasmic calcium binding protein, troponin C or Triose-phosphate isomerase or actin. In some embodiments, the allergen is Pan b 1. In some embodiments the antigenic unit comprises Pan b 1 T cell epitope (251-270). In some embodiment, the antigenic unit comprises Met e 1. In some embodiment, the antigenic unit comprises one or more of the Met e 1 T cell epitopes (241-260), (210-230), (136-155), (76-95), (46-65) and (16-35). In some embodiments, the antigenic unit comprises all of the Met e 1 T cell epitopes (241-260), (210-230), (136-155), (76-95), (46-65) and (16-35).

In some embodiments, the allergen is a cow's milk allergen. In some embodiments, the cow's milk allergen is Bos d 4, Bos d 5, Bos d 6, Bos d 7, Bos d 8, Bos d 9, Bos d 10, Bos d 11 or Bos d 12.

In some embodiments, the allergen is an egg allergen. In some embodiments, the egg allergen is ovomucoid, in other embodiments the egg allergen is ovalbumin, ovotransferin, conalbumin, Gal d 3, egg lysozyme or ovomucin.

One T cell epitope that is known in the art and has been studied in the context of egg allergy is OVA (257-264), with amino acid sequence SIINFEKL (SEQ ID NO: 120).

In one embodiment, the antigenic unit of the construct encoded in the vector of the invention comprises the T cell epitope OVA (257-264). Such vector or a pharmaceutical composition comprising such vector may be used in the treatment of egg allergy.

In some embodiments, the allergen is a fish allergen. In some embodiments, the fish allergen is a parvalbumin. In other embodiments the fish allergen is enolase, aldolase or vitellogenin.

In some embodiments, the allergen is a fruit allergen. In some embodiments, the fruit allergen is pathogenesis-related protein 10, profilin, nsLTP, thaumatin-like protein, gibberellin regulated protein, isoflavone reductase related protein, class 1 chitinase, β 1,3 glucanase, germin like protein, alkaline serine protease, pathogenesis-related protein 1, actinidin, phytocystatin, kiwellin, major latex protein, cupin or 2S albumin. In some embodiments, the allergen is a vegetable allergen. In some embodiments, the vegetable allergen is pathogenesis related protein 10, profilin, nsLTP type 1, nsLTP type protein 2, osmotin-like protein, isoflavone reductase-like protein, β-fructofuranosidase, PR protein TSI-1, cyclophilin or FAD containing oxidase.

In some embodiments, the allergen is a wheat allergen. In some embodiments, the wheat allergen is Tri a 12, Tri a 14, Tri a 15, Tri a 18, Tri a 19, Tri a 20, Tri a 21, Tri a 25, Tri a 26, Tri a 27, Tri a 28, Tri a 29, Tri a 30, Tri a 31, Tri a 32, Tri a 33, Tri a 34, Tri a 35, Tri a 36, Tri a 37 or Tri a 38. In some embodiments, the allergen is a soy allergen. In some embodiments, the soy allergen is Gly m 1, Gly m 2, Gly m 3, Gly m 4, Gly m 5, Gly m 6, Gly m 7 or Gly m 8. In other embodiments the soy allergen is Gly m agglutinin, Gly m Bd28K, Gly m 30 kD, Gly m CPI or Gly m TI.

In some embodiments, the allergen is a peanut allergen. In some embodiments, the peanut allergen is Ara h 1, Ara h 2, Ara h 3, Ara h 4, Ara h 5, Ara h 6, Ara h 7, Ara h 8, Ara h 9, Ara h 10, Ara h 11, Ara h 12, Ara h 13, Ara h 14, Ara h 15, Ara h 16, or Ara h 17. In some embodiments, the allergen is a tree nut or seed allergen. In some embodiments, the allergen is 11S globulin, 7S globulin, 2S globulin, PR10, PR-14 nsLTP, oleosin or profilin.

In other embodiments the food allergen is an allergen selected from the group consisting of buckwheat, celery, a color additive, egg, fish, fruit, garlic, gluten, oats, legumes, maize, milk, mustard, nuts, peanuts, poultry, meat, rice, sesame, shellfish, soy, tree nut and wheat.

In some embodiments, the allergen is a bee venom allergen. In some embodiments, the bee venom allergen is Phospholipase A2, Hyaluronidase, acid phosphatase, melittin, allergen C/DPP, CRP/icarapin or vitellogenin. In some embodiments, the allergen is a vespid allergen. In some embodiments, the vespid allergen is Phospholipase A1, hyaluronidase, protease, antigen 5, DPP IV or vitellogenin.

In some embodiments, the allergen is a latex allergen. In some embodiments, the latex allergen is Hev b 1, Hev b 2, Hev b 3, Hev b 4, Hev b 5, Hev b 6, Hev b 7, Hev b 8, Hev b 9, Hev b 10, Hev b 11, Hev b 12, Hev b 13, Hev b 14 or Hev b 15.

In some embodiments, the allergen is a dust mite allergen. In some embodiments the allergen is a house dust mite allergen. In some embodiments, the allergen is a storage dust allergen. In some embodiments, the house dust mite allergen is Der p 1, Der p 2, Der p 3, Der p 4, Der p 5, Der p 7, Der p 8, Der p 10, Der p 11, Der p 21, or Der p 23. In some embodiments the antigenic unit comprises the Der p 1 T cell epitope (111-139). In some embodiments, the house dust mite allergen is Der f 1, Der f 2, Der f 3, Der f 7, Der f 8 or Der f 10. In some embodiments, the house dust mite allergen is Blot t 1, Blot t 2, Blot t 3, Blot t 4, Blot t 5, Blot t 8, Blot t 10, Blot t 12 or Blot t 21.

In some embodiments, the allergen is a cockroach allergen. In some embodiments, the cockroach allergen is Bla g 1, Bla g 2, Bla g 3, Bla g 4, Bla g 5, Bla g 6, Bla g 7, Bla g 8 or Bla g 11. In some embodiments, the cockroach allergen is Per a 1, Per a 2, Per a 3, Per a 6, Per a 7, Per a 9 or Per a 10.

In some embodiments, the allergen is a mold allergen. In some embodiments, the mold allergen is an Aspergillus fumigatus allergen. In some embodiments, the Aspergillus fumigatus allergen is Asp f 1, Asp f 2, Asp f 3, Asp f 4, Asp f 5, Asp f 6, Asp f 7, Asp f 8, Asp f 9, Asp f 10, Asp f 11, Asp f 12, Asp f 13, Asp f 14, Asp f 15, Asp f 16, Asp f 17, Asp f 18, Asp f 22, Asp f 23, Asp f 27, Asp f 28, Asp f 29 or Asp f 34.

In some embodiments, the allergen is a fungal allergen. In some embodiments, the fungal allergen is a Malassezia allergen. In some embodiments, the Malassezia allergen is Mala f 1, Mala f 2, Mala f 3, Mala f 4, Mala f 5, Mala f 6, Mala f 7, Mala f 8, Mala f 9, Mala f 10, Mala f 11, Mala f 12 or Mala f 13 or MGL_1204.

In some embodiments, the allergen is furry animal allergen. In some embodiments, the allergen is a dog allergen. In some embodiments, the dog allergen is Can f 1, 2, 3, 4, 5, or 6. In some embodiments, the allergen is a horse allergen. In some embodiments, the horse allergen is Ecu c 1, 2, 3 or 4. In some embodiments, the allergen is a cat allergen. In some embodiments, the cat allergen is Fel d 1, Fel d 2, Fel d 3, Fel d 4, Fel d 5, Fel d 6, Fel d 7, or Fel d 8. In some embodiments, the allergen is a laboratory animal allergen. In some embodiments, the allergen is lipocalin, urinary prealbumin, secretoglobulin or serum albumin.

In some embodiments, the allergen is a pollen allergen. In some embodiments, the allergen is a grass pollen allergen. In some embodiments, the grass pollen allergen is a timothy grass, orchard grass, Kentucky bluegrass, perennial rye, sweet vernal grass, bahia grass, johnson grass or Bermuda grass allergen. In some embodiments the grass pollen allergen is Phl p 1, Phl p 2, Phi p 3, Phi p 4, Phi p 5, Phl p 6, Phl p 7, Phi p 11, Phl p 12 or Phl p 13.

In some embodiments, the allergen is a tree pollen allergen. In some embodiments, the tree pollen allergen is a alder, birch, hornbeam, hazel, European hophornbeam, chestnut, European beech, white oak, ash, privet, olive, lilac, cypress or cedar pollen allergen. In some embodiments, the tree pollen allergen is Aln g 1 or Aln g 4, Bet v 1, Bet v 2, Bet v 3, Bet v 4, Bet v 6 or Bet v 7, Car b 1, Cor a 1, Cor a 2, Cor a 6, Cor a 8, Cor a 9, Cor a 10, Cor a 11, Cor a 12, 1 Cor a 3, Cor a 14, Ost c 1, Cas 1, Cas 5, Cas 8 or Cas 9, Fag s 1, Que a 1, Fra e 1, Lig v 1, Ole e 1, Ole e 2, Ole e 3, Ole e 4, Ole e 5, Ole e 6, Ole e 7, Ole e 8, Ole e 9, Ole e 10, Ole e 11 or Ole e 12, Syr v 1, Cha o 1, Cha o 2, Cry j 1, Cry j 2, Cup s 1, Cup s 3, Jun a 1, Jun a 2, Jun a 3, Jun o 4, Jun v 1, Jun v 3, Pla a 1, Pla a 2 or Pla a 3. In some embodiments, the antigenic unit comprises the Bet v 1 T cell epitope (139-152).

In some embodiments, the allergen is a weed pollen allergen. In some embodiments the weed allergen is a ragweed, mugwort, sunflower, feverfew, pellitory, English plantain, annual mercury, goosefoot, Russian thistle or amaranth pollen allergen. In some embodiments the ragweed pollen allergen is Amb a 1, Amb a 4, Amb a 6, Amb a 8, Amb a 9, Amb a 10, or Amb a 11. In some embodiments the mugwort pollen allergen is Art v 1, Art v 3, Art v 4, Art v 5, or Art v 6. In some embodiments, the sunflower pollen allergen is Hel a 1 or Hel a 2. In some embodiments, the pellitory pollen allergen is Par j 1, Par j 2, Par j 3 or Par j 4. In some embodiments, the English plantain pollen allergen is Pla l 1. In some embodiments, the annual mercury pollen allergen is Mer a 1. In some embodiments, the goosefoot pollen allergen is Che a 1, Che a 2 or Che a 3. In some embodiments, the Russian thistle pollen allergen is Sal k 1, Sal k 4 or Sal k 5. In some embodiments, the Amaranth pollen allergen is Ama r 2.

In yet other embodiments the allergen is selected form environmental allergens such insects, cockroaches, house dust mites or mold.

In some embodiments, the vectors of the invention may be used in the treatment of allergic diseases selected from the group consisting of allergic rhinitis, asthma, atopic dermatitis, allergic gastroenteropathy, contact dermatitis and drug allergy or combinations thereof.

Allergy to drugs affect more than 7% of the general population. The constructs encoded by the vectors of the invention may be used to induce tolerance towards immunogenic T cell epitopes present in such a drug and thus will allow affected patients to continue treatment with the drug and receive the benefits from the drug treatment.

Thus, in some embodiments, the allergen is comprised in a drug with unwanted immunogenicity. In some embodiments, the allergen is Factor VIII. In some embodiments, the allergen is insulin. In some embodiments, the allergen is a monoclonal antibody used for therapy.

### Self-Antigens

In another embodiment, the present vector encodes a construct that contains one or more T cell epitopes comprised in a self-allergen that is involved in an autoimmune disease. This allows for the antigen-specific down-regulation of the part of the immune system responsible for the autoimmune disease without inhibiting the immune system in general.

In some embodiments, the autoimmune disease is MS. In some embodiments, the self-antigen is myelin oligodendrocyte glycoprotein (MOG). In other embodiments the self-antigen is MAG, MOBP, CNPase, S100β or transaldolase. In some embodiments, the self-antigen is myelin basic protein (MBP). In some embodiments, the self-antigen is myelin proteolipid protein (PLP). In some embodiments, the construct encoded by the vector of the invention comprises one or more T cell epitopes derived from one or more of the aforementioned self-antigens.

MS-relevant T cell epitopes are T cell epitopes from myelin oligodendrocyte glycoprotein (MOG). MOG is a member of the immunoglobulin superfamily and is expressed exclusively in the central nervous system. MOG (35-55) is able to induce autoantibody production and relapsing-remitting neurological disease, causing extensive plaque-like demyelination. Autoantibody response to MOG (35-55) has been observed in MS patients and MOG (35-55)-induced experimental autoimmune encephalomyelitis (EAE) in C57/BL6 mice and Lewis rats. Another MOG T cell epitope is MOG (27-63).

Other MS-relevant T cell epitopes that are known in the art and have been studied include the following listed in Table 3:

TABLE 3

| T cell epitope | Sequence | SEQ ID NOS |
|---|---|---|
| PLP (139-151)* | HCLGKWLGHPDKF | SEQ ID NO: 167 |
| PLP (131-159) | AHSLERVCHCLGKWLGHPDKFVGITYALT | SEQ ID NO: 168 |
| PLP (178-191)* | NTWTTCQSIAFPSK | SEQ ID NO: 169 |
| PLP (170-199) | AVPVYIYFNTWTTCQSIAFPSKTSASIGSL | SEQ ID NO: 170 |
| MBP (84-104)* | VHFFKNIVTPRTPPPSQGKGR | SEQ ID NO: 171 |
| MBP (76-112) | RTQDENPVVHFFKNIVTPRTPPPSQGKGRGLSLSRF | SEQ ID NO: 172 |

### *T Cell Epitope-Induced EAE Observed

In a preferred embodiment, the antigenic unit of the construct encoded by the vector of the invention includes one or more T cell epitopes selected from the group consisting of MOG (35-55), MOG (27-63), PLP (139-151), PLP (131-159), PLP (178-191), PLP (170-199), MBP (84-104) and MBP (76-112). Such vector or a pharmaceutical composition comprising such vector may be used in the treatment of MS.

In some embodiments, the autoimmune disease is type 1 diabetes mellitus. In some embodiments, the self-antigen is glutamic acid decarboxylase 65-kilodalton isoform (GAD65), which is a self-antigen involved in type 1 diabetes mellitus. In some other embodiments, the self-antigen is insulin, IA-2 or ZnT8. In yet some other embodiments, the self-antigen is IGRP, ChgA, IAPP, peripherin, tetraspanin-7, GRP78, urocortin-3 or insulin gene enhancer protein isl-1. In some embodiments, the construct encoded by the vector of the invention comprises one or more T cell epitopes derived from one or more of the aforementioned self-antigens

In some embodiments, the autoimmune disease is celiac disease. In some embodiments, the self-antigen is α-gliadin, γ-gliadin, ω-gliadin, low molecular weight glutenin, high molecular weight glutenin, hordein, secalin or avenin b. In some embodiments, the antigenic unit comprises the T cell epitope α-gliadin (76-95). In some embodiments, the construct encoded by the vector of the invention comprises one or more T cell epitopes derived from one or more of the aforementioned self-antigens.

In some embodiments, the autoimmune disease is rheumatoid arthritis. In some embodiments, the self-antigen is collagen. In some embodiments, the self-antigen is heat shock protein 60 (HSP60). In some embodiments, the self-antigen is Band 3. In some embodiments, the self-antigen is small nuclear ribonucleoprotein D1 (SmD1). In some embodiments, the self-antigen is the acetylcholine receptor (AChR). In some embodiments, the self-antigen is myelin protein zero (P0). In some embodiments, the construct encoded by the vector of the invention comprises one or more T cell epitopes derived from one or more of the aforementioned self-antigens

In one embodiment, the autoimmune disease is chronic inflammatory demyelinating polyradiculoneuropathy (CIDP) and the self-antigen is neurofascin 155. In another embodiment, the autoimmune disease is Hashimoto's thyroiditis (HT) and the self-antigen is thyroid peroxidase and/or thyroglobulin. In another embodiment, the autoimmune disease is Pemphigus foliaceus and the self-antigen is desmosome-associated glycoprotein. In another embodiment, the autoimmune disease is Pemphigus vulgaris and the self-antigen is desmoglein 3. In another embodiment, the autoimmune disease is thyroid eye disease (TED) and the self-antigen is calcium binding protein (calsequestrin). In another embodiment, the autoimmune disease is Grave's disease and the self-antigen is thyroid stimulating hormone receptor. In another embodiment, the autoimmune disease is primary biliary cirrhosis (PBC) and the self-antigen is antimitochondrial antibodies (AMAs), antinuclear antibodies (ANA), Rim-like/membrane (RL/M) and/or multiple nuclear dot (MND). In another embodiment, the autoimmune disease is myasthenia gravis and the self-antigen is acetylcholine receptor. In another embodiment, the autoimmune disease is insulin-resistant diabetes and the self-antigen is insulin receptor. In another embodiment, the autoimmune disease is autoimmune hemolytic anemia and the self-antigen is erythrocytes. In another embodiment, the autoimmune disease is psoriasis and the self-antigen is selected from the group consisting of cathelicidin (LL-37), disintegrin-like and metalloprotease domain containing thrombospondin type 1 motif-like 5 (ADAMTSL5), phospholipase A2 group IVD (PLA2G4D), heterogeneous nuclear ribonucleoprotein A1 (hnRNP-A1) and keratin 17. In another embodiment, the autoimmune disease is rheumatoid arthritis and the self-antigen is selected from the group consisting of citrullinated proteins, homocitrullinated proteins and the Fc portion of IgG. In some embodiments, the construct encoded by the vector of the invention comprises one or more T cell epitopes derived from one or more of the aforementioned self-antigens.

### Unit Linker

The antigenic unit is connected to the multimerization unit, preferably by a unit linker. Thus, in one embodiment, the first nucleic acid sequence comprised in the vectors of the invention encodes a unit linker that connects the antigenic unit to the multimerization unit.

The unit linker may comprise a restriction site in order to facilitate the construction of the first nucleic acid sequence. In one embodiment, the unit linker is GLGGL (SEQ ID NO: 102) or GLSGL (SEQ ID NO: 174). In another embodiment, the unit linker comprises or consists of GGGGS (SEQ ID NO: 175), GGGGSGGGGS (SEQ ID NO: 79), (GGGGS)m (SEQ ID NO: 80), EAAAK (SEQ ID NO: 176), (EAAAK)m (SEQ ID NO: 154), (EAAK)mGS (SEQ ID NO: 178), (EAAAK)mGS (SEQ ID NO: 177), GPSRLEEELRRRLTEPG (SEQ ID NO: 179), AAY or HEYGAEALERAG (SEQ ID NO: 173).

### Signal Peptide

In one embodiment of the present disclosure, at least one of the first nucleic acid sequence and the one or more further nucleic acid sequences encoding one or more immunoinhibitory compounds also encodes a signal peptide. The signal peptide is either located at the N-terminal end of the targeting unit or the C-terminal end of the targeting unit, depending on the orientation of the targeting unit in the first polypeptide. Further, the signal peptide is located at the N-terminal end of the immunoinhibitory compound. The signal peptide is designed to allow secretion of the first polypeptide and/or the immunoinhibitory compound(s) from the cells comprising the vector of the invention. Preferably, the first nucleic acid sequence and each of the further nucleic acid sequence encoding one or more immunoinhibitory compounds also encode a signal peptide.

Any suitable signal peptide may be used. For the first polypeptide, an example of a suitable signal peptide is a human Ig VH signal peptide, preferably if the targeting unit is an antibody or part thereof, such as a scFv. In one embodiment, the signal peptide is the natural leader sequence of the protein which is the targeting unit, i.e. the signal peptide which is naturally present at the N-terminus of the protein which is encoded in the vector of the invention as the targeting unit. Examples of such signal peptides are the signal peptide of human IL-10 (with the targeting unit being human IL-10) or the signal peptide of human TGF-β1 (with the targeting unit being human TGF-β1).

For the one or more immunoinhibitory compounds, the signal peptide is preferably the natural leader sequence of the immunoinhibitory compound, i.e. the signal peptide which is naturally present at the N-terminus of the immunoinhibitory compound. Examples of such signal peptides are the signal peptide of CTLA-4 (with the immunoinhibitory compound being CTLA-4, preferably the extracellular domain of CTLA-4) or the signal peptide of GM-CSF (with the immunoinhibitory compound being GM-CSF).

Thus, in one embodiment, the vector of the invention comprises a first nucleic acid sequence that encodes a human IL-10 signal peptide, such as the human IL-10 signal with SEQ ID NO: 69. In a preferred embodiment, such vector comprises a first nucleic acid sequence that encodes a human IL-10 targeting unit and also encodes a human IL-10 signal peptide. In another embodiment, the vector of the invention comprises a first nucleic acid sequence that encodes a human Ig VH signal peptide, such as a human Ig VH signal peptide with SEQ ID NO: 2. In a preferred embodiment, such vector comprises a first nucleic acid sequence that encodes a scFv targeting unit, e.g. an anti-human CD205 targeting unit and also encodes a human Ig VH signal peptide.

In one embodiment, the vector of the invention comprises a first nucleic acid sequence that encodes a signal peptide selected from the group consisting of human Ig VH signal peptide, signal peptide of hTGF-β1, signal peptide of hTGF-β2 signal peptide of hTGF-β3, signal peptide of hIL-10, signal peptide of hIL-2, signal peptide of hIL-4, signal peptide of hIL-6, signal peptide of hIL-11, signal peptide of hIL-13, signal peptide of hIL-27, signal peptide of hIL-35, signal peptide of hIL-37, signal peptide of hGM-CSF, signal peptide of hFLT3L, signal peptide of hCCL19, signal peptide of hCCL21, signal peptide of hICAM-1, signal peptide of hkeratin, signal peptide of hVSIG-3, signal peptide of hSCGB3A2, signal peptide of hCTLA-4, signal peptide of hPD-1 and signal peptide of hBTLA, such as any of the aforementioned signal peptides as listed in the section “Sequence overview” herein.

In another embodiment, the vector of the invention comprises one or more further nucleic acid sequences which encode one or more immunoinhibitory compounds and further encode a signal peptide selected from the group consisting of signal peptide of hCLTA-4, signal peptide of hPD-1, signal peptide of hBTLA, signal peptide of hLAG3, signal peptide of hNOX2, signal peptide of hSIGLEC7, signal peptide of hSIGLEC9, signal peptide of hTIM-3, signal peptide of hIL-10, signal peptide of hTGF-β1, signal peptide of hTGF-β2, signal peptide of hTGF-β3, signal peptide of hIL-27, signal peptide of hIL-2, signal peptide of hGM-CSF signal peptide of hFLT3L, signal peptide of hIFN-γ and signal peptide of hIL-37 and signal peptide of hIL-35, such as any of the aforementioned signal peptides as listed in the section “Sequence overview” herein.

### Sequence Identity

Sequence identity may be determined as follows: A high level of sequence identity indicates likelihood that a second sequence is derived from a first sequence. Amino acid sequence identity requires identical amino acid sequences between two aligned sequences. Thus, a candidate sequence sharing 70% amino acid identity with a reference sequence requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. Using this program with its default settings, the mature (bioactive) part of a query and a reference polypeptide are aligned. The number of fully conserved residues is counted and divided by the length of the reference polypeptide. In doing so, any tags or fusion protein sequences, which form part of the query sequence, are disregarded in the alignment and subsequent determination of sequence identity.

The ClustalW algorithm may similarly be used to align nucleotide sequences. Sequence identities may be calculated in a similar way as indicated for amino acid sequences.

Another preferred mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment. Align0 does not penalize to gaps in the end of the sequences. When utilizing the ALIGN and Align0 program for comparing amino acid sequences, a BLOSUM50 substitution matrix with gap opening/extension penalties of −12/−2 is preferably used

Amino acid sequence variants may be prepared by introducing appropriate changes into the nucleotide sequence encoding the first polypeptide and/or one or more immunostimulatory compounds, or by peptide synthesis. Such modifications include, for example, deletions from, and/or insertions into and/or substitutions of, residues within the amino acid sequences. The terms substituted/substitution, deleted/deletions and inserted/insertions as used herein in reference to amino acid sequences and sequence identities are well known and clear to the skilled person in the art. Any combination of deletion, insertion, and substitution can be made to arrive at the final first polypeptide and/or one or more immunostimulatory compounds, provided that the final proteins have the desired characteristics. For example, deletions, insertions or substitutions of amino acid residues may produce a silent change and result in a functionally equivalent polypeptide/immunostimulatory compound.

Deliberate amino acid substitutions may be made on the basis of similarity in polarity, charge, solubility, hydrophobicity, hydrophilicity, and/or the amphipathic nature of the residues as long as the desired properties of the protein in question are retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Herein encompassed are conservative substitutions, i.e. like-for-like substitution such as basic for basic, acidic for acidic, polar for polar etc. and non-conservative substitutions, i.e. from one class of residue to another or alternatively involving the inclusion of unnatural amino acids such as ornithine, diaminobutyric acid ornithine, norleucine, ornithine, pyriylalanine, thienylalanine, naphthylalanine and phenylglycine. Conservative substitutions that may be made are, for example within the groups of basic amino acids (arginine, lysine and histidine), acidic amino acids (glutamic acid and aspartic acid), aliphatic amino acids (alanine, valine, leucine, isoleucine), polar amino acids (glutamine, asparagine, serine, threonine), aromatic amino acids (phenylalanine, tryptophan, tyrosine), hydroxyl amino acids (serine, threonine), large amino acids (phenylalanine, tryptophan) and small amino acids (glycine, alanine).

Substitutions may also be made by unnatural amino acids and substituting residues include alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, 3-alanine*, L-a-amino butyric acid*, L-y-amino butyric acid*, L-a-amino isobutyric acid*, L-e-amino caproic acid*, 7-amino heptanoic acid*, L-methionine sulfone*, L-norleucine*, L-norvaline*, p-nitro-L-phenylalanine*, L-hydroxyproline*, L-thioproline*, methyl derivatives of phenylalanine (Phe) such as 4-methyl-Phe*, pentamethyl-Phe*, L-Phe (4-amino) #, L-Tyr (methyl)*, L-Phe (4-isopropyl)*, L-Tic (1,2,3,4-tetrahydroisoquinoline-3-carboxyl acid)*, L-diaminopropionic acid* and L-Phe (4-benzyl)*.

In the paragraph above,* indicates the hydrophobic nature of the substituting residue, whereas # indicates the hydrophilic nature of substituting residue and #* indicates amphipathic characteristics of the substituting residue. Variant amino acid sequences may include suitable spacer groups that may be inserted between any two amino acid residues of the sequence including alkyl groups such as methyl, ethyl or propyl groups in addition to amino acid spacers such as glycine or B-alanine residues. A further form of variation involves the presence of one or more amino acid residues in peptoid form.

### Polypeptides and Multimeric/Dimeric Proteins

The vectors of the invention encode a first polypeptide as described above. The polypeptide (and the one or more immunoinhibitory compounds) are expressed in vivo as a result of the administration of the vector to a subject.

Due to the presence of the multimerization unit, such as dimerization unit, multimeric proteins are formed when the polypeptide is expressed.

The multimeric proteins may be homomultimers or hetereomultimers, e.g. if the protein is a dimeric protein, the dimeric protein may be a homodimer, i.e. a dimeric protein wherein the two polypeptide chains are identical and consequently comprise identical units and thus T cell epitopes, or the dimeric protein may be a heterodimer comprising two polypeptide chains, wherein polypeptide chain 1 comprises different T cell epitopes in its antigenic unit than polypeptide 2. The latter may be relevant if the number of T cell epitopes for inclusion into the antigenic unit would exceed an upper size limit for the antigenic unit. It is preferred that the multimeric protein is a homomultimeric protein.

### Host Cells and Preparation of the Vector

The vectors of the invention are generally vectors suitable for transfecting a host cell and a) expression of the first polypeptide and formation of a multimeric protein comprised of multiple of such first polypeptides encoded by the first nucleic acid sequence and b) expression of the one or more immunoinhibitory compounds encoded by the further nucleic acid sequences, respectively.

In one embodiment, the host cell comprising the vector of the invention is a cell of a cell culture, e.g. a bacteria cell, and the proteins encoded by the vector are expressed in vitro. In another embodiment, the host cell comprising the vector of the invention is a cell of a subject and the proteins encoded by the vector are expressed in said subject, i.e. in vivo, as a result of the administration of the vector to a subject.

Suitable host cells for in vitro transfection include prokaryote cells, yeast cells, insect cells or higher eukaryotic cells. Suitable host cells for in vivo transfection are e.g. muscle cells.

In one embodiment, the vector is a CpG-free vector. In another one embodiment, the vector is a pALD-CV77 vector.

Engineering and production methods of the vectors of the invention, e.g. expression vectors such as DNA and RNA plasmids or viral vectors are well known and the skilled person will be able to engineer/produce the vectors of the invention using such known methods. Moreover, various commercial manufacturers offer services for vector design and production.

In one aspect, the disclosure relates to a method of producing a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of a self-antigen, an allergen, an alloantigen or a xenoantigen; and
(b) one or more further nucleic acid sequences encoding one or more immunoinhibitory compounds,
wherein the vector allows for the co-expression of the first polypeptide and the one or more immunoinhibitory compounds as separate molecules, the method comprising:
   a) transfecting cells in vitro with the vector;
   b) culturing said cells;
   c) optionally, lysing the cells to release the vector from the cells; and
   d) collecting and optionally purifying the vector or the DNA plasmid.

### Pharmaceutical Compositions

In one embodiment of the present disclosure, the vector, e.g. DNA plasmid is for use as a medicament.

Thus, in one embodiment of the present disclosure, the vector is provided in a pharmaceutical composition comprising the vector and a pharmaceutically acceptable carrier or diluent.

Thus, in one aspect, the disclosure relates to a pharmaceutical composition comprising (i) a pharmaceutically acceptable carrier or diluent and (ii) a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of a self-antigen, an allergen, an alloantigen or a xenoantigen; and
(b) one or more further nucleic acid sequences encoding one or more immunoinhibitory compounds,
wherein the vector allows for the co-expression of the first polypeptide and the one or more immunoinhibitory compounds as separate molecules.

Suitable pharmaceutically acceptable carriers or diluents include, but are not limited to, saline, buffered saline, such as PBS, dextrose, water, glycerol, ethanol, isotonic aqueous buffers, and combinations thereof.

In one embodiment, the pharmaceutically acceptable carrier or diluent is an aqueous buffer. In another embodiment, the aqueous buffer is Tyrode's buffer, e.g. Tyrode's buffer comprising 140 mM NaCl, 6 mM KCl, 3 mM CaCl2, 2 mM MgCl2, 10 mM 4-(2-hydroxyethyl)-1-piperazineethanesulfonic acid (Hepes) pH 7.4, and 10 mM glucose.

The pharmaceutical composition may comprise molecules that ease the transfection of host cells, i.e. a transfection agent.

The pharmaceutical composition may comprise an adjuvant. In one embodiment, the adjuvant is selected from the group consisting of dexamethasone, B subunits of enterotoxin cholera toxin (CTB), TLR2 ligands, helminth-derived excretory/secretory (ES) products, rapamycin vitamin D3 analogues and aryl hydrocarbon receptor ligands.

In some specific embodiments pharmaceutical composition comprises a pharmaceutically acceptable amphiphilic block co-polymer comprising blocks of poly(ethylene oxide) and polypropylene oxide).

An “amphiphilic block co-polymer” as used herein is a linear or branched co-polymer comprising or consisting of blocks of poly(ethylene oxide) (“PEO”) and blocks of poly(propylene oxide) (“PPO”). Typical examples of useful PEO-PPO amphiphilic block co-polymers have the general structures PEO-PPO-PEO (poloxamers), PPO PEO PPO, (PEO PPO-)4ED (a poloxamine), and (PPO PEO-)4ED (a reverse poloxamine), where “ED” is a ethylenediaminyl group.

A “poloxamer” is a linear amphiphilic block co-polymer constituted by one block of poly(ethylene oxide) coupled to one block of poly(propylene oxide) coupled to one block of PEO, i.e. a structure of the formula EOa-POb-EOa, where EO is ethylene oxide, PO is propylene oxide, a is an integer from 2 to 130, and b is an integer from 15 to 67. Poloxamers are conventionally named by using a 3-digit identifier, where the first 2 digits multiplied by 100 provides the approximate molecular mass of the PPO content, and where the last digit multiplied by 10 indicates the approximate percentage of PEO content. For instance, “Poloxamer 188” refers to a polymer comprising a PPO block of a molecular weight of about 1800 (corresponding to b being about 31 PPO) and approximately 80% (w/w) of PEO (corresponding to a being about 82). However, the values are known to vary to some degree, and commercial products such as the research grade Lutrol® F68 and the clinical grade Kolliphor® P188, which according to the producer's data sheets both are Poloxamer 188, exhibit a large variation in molecular weight (between 7,680 and 9,510) and the values for a and b provided for these particular products are indicated to be approximately 79 and 28, respectively. This reflects the heterogeneous nature of the block co-polymers, meaning that the values of a and b are averages found in a final formulation.

A “poloxamine” or “sequential poloxamine” (commercially available under the trade name of Tetronic®) is an X-shaped block co-polymers that bears four PEO-PPO arms connected to a central ethylenediamine moiety via bonds between the free OH groups comprised in the PEO-PPO-arms and the primary amine groups in ethylenediamine moiety. Reverse poloxamines are likewise X-shaped block co-polymers that bear four PPO-PEO arms connected to a central ethylenediamine moiety via bonds between the free OH groups comprised in the PPO-PEO arms and the primary amine groups in ethylenediamine.

Preferred amphiphilic block co-polymers are poloxamers or poloxamines. Preferred are poloxamer 407 and 188, in particular poloxamer 188. Preferred poloxamines are sequential poloxamines of formula (PEO-PPO)4-ED. Particularly preferred poloxamines are those marketed under the registered trademarks Tetronic® 904, 704, and 304, respectively. The characteristics of these poloxamines are as follows: Tetronic® 904 has a total average molecular weight of 6700, a total average weight of PPO units of 4020, and a PEO percentage of about 40%. Tetronic® 704 has a total average molecular weight of 5500, a total average weight of PPO units of 3300, and a PEO percentage of about 40%; and Tetronic® 304 has a total average molecular weight of 1650, a total average weight of PPO units of 990, and a PEO percentage of about 40%.

In one embodiment, the pharmaceutical composition comprises the amphiphilic block co-polymer in an amount of from 0.2% w/v to 20% w/v, such as of from 0.2% w/v to 18% w/v, 0.2% w/v to 16% w/v, 0.2% w/v to 14% w/v, 0.2% w/v to 12% w/v, 0.2% w/v to 10% w/v, 0.2% w/v to 8% w/v, 0.2% w/v to 6% w/v, 0.2% w/v to 4% w/v, 0.4% w/v to 18% w/v, 0.6% w/v to 18% w/v, 0.8% w/v to 18% w/v, 1% w/v to 18% w/v, 2% w/v to 18% w/v, 1% w/v to 5% w/v, or 2% w/v to 4% w/v. Particularly preferred are amounts in the range of from 0.5% w/v to 5% w/v. In another embodiment, the pharmaceutical composition comprises the amphiphilic block co-polymer in an amount of from 2% w/v to 5% w/v, such as about 3% w/v.

The pharmaceutical composition may be formulated in any way suitable for administration to a subject, e.g. such as a liquid formulation for injection, e.g. for intradermal or intramuscular injection.

The pharmaceutical composition may be administered in any way suitable for administration to a subject, such as administered by intradermal, intramuscular, or subcutaneous injection, or by mucosal or epithelial application, such as intranasal or oral.

In a preferred embodiment, the pharmaceutical composition is administered by intramuscular or intradermal injection.

The amount of vector, e.g. DNA plasmid, in the pharmaceutical composition may vary depending on whether the pharmaceutical composition is administered for prophylactic or therapeutic treatment.

The pharmaceutical composition of the invention typically comprises the vector, e.g. DNA plasmid, in a range of from 0.1 to 10 mg, e.g. about 0.1, 0.2, 0.3, 0.4, 0.5, 0.6, 0.7, 0.8, 0.9 or 1 mg or e.g. 2, 3, 4, 5, 6, 7, 8, 9 or 10 mg.

In a preferred embodiment, the pharmaceutical composition is a sterile pharmaceutical composition.

### Treatment

In some aspects of the present disclosure, the vector, e.g. DNA plasmid, is for use in the prophylactic or therapeutic treatment of conditions involving undesirable immune reactions, i.e. immune diseases, such as in the prophylactic or therapeutic treatment of autoimmune diseases, allergic disease and graft rejection.

Thus, in a further aspect, the invention provides a method for treating a subject having an immune disease selected from the group consisting of autoimmune disease, allergic disease and graft rejection or being in need of prevention thereof, the method comprising administering to the subject a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of a self-antigen, an allergen, an alloantigen or a xenoantigen; and
(b) one or more further nucleic acid sequences encoding one or more immunoinhibitory compounds, wherein the vector allows for the co-expression of the first polypeptide and the one or more immunoinhibitory compounds as separate molecules.

In one embodiment, the invention provides a method for treating a subject having an autoimmune disease or being in need of prevention thereof, the method comprising administering to the subject a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of a self-antigen; and
(b) one or more further nucleic acid sequences encoding one or more immunoinhibitory compounds, wherein the vector allows for the co-expression of the first polypeptide and the one or more immunoinhibitory compounds as separate molecules.

In another embodiment, the invention provides a method for treating a subject having an allergic disease or being in need of prevention thereof, the method comprising administering to the subject a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of an allergen; and
(b) one or more further nucleic acid sequences encoding one or more immunoinhibitory compounds, wherein the vector allows for the co-expression of the first polypeptide and the one or more immunoinhibitory compounds as separate molecules.

In one embodiment, the invention provides a method for treating a subject having a graft rejection or being in need of prevention thereof, the method comprising administering to the subject a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of an alloantigen or xenoantigen; and
(b) one or more further nucleic acid sequences encoding one or more immunoinhibitory compounds,
wherein the vector allows for the co-expression of the first polypeptide and the one or more immunoinhibitory compounds as separate molecules.

In the method of treatment, the vector is preferably administered in a therapeutically effective or prophylactically effective amount. Such amount of vector may be administered in one administration, i.e. one dose, or in several administrations, i.e. repetitive doses, i.e. in a series of doses, e.g. over the course of several days, weeks or months or years.

The actual dose to be administered may vary and depend on whether the treatment is a prophylactic or therapeutic treatment, the severity of the immune disease being treated, on parameters like the age, weight, gender, medical history, pre-existing conditions and general condition of the subject and the judgment of the health care professionals.

In the method of treatment, the vector may be administered in the form of the pharmaceutical composition and in the mode of administration as described herein.

The method of treating according to the invention can continue for as long as the clinician overseeing the patient's care deems the method to be effective and the treatment to be needed.

Indicators of treatment success are known in the art, including increased levels of antigen-specific regulatory T cells, reduced levels of antigen-specific effector T cells, (and increased levels of regulatory T cells), reduced levels of effector T cells, reduced level of T cell activation in ELISPOT when stimulated with the antigenic unit/T-cell epitopes in the antigenic unit, reduced level of basophil activation in a basophil activation test (BAT).

A radioallergosorbent test (RAST) may likewise be used to compare the allergen-specific IgE antibody level in a blood sample from a subject before and after administration of the immunotherapy construct, wherein a lower allergen-specific IgE antibody level indicates successful tolerance induction.

Also disclosed herein is a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of a self-antigen, an allergen, an alloantigen or a xenoantigen; and
(b) one or more further nucleic acid sequences encoding one or more immunoinhibitory compounds,
wherein the vector allows for the co-expression of the first polypeptide and the one or more immunoinhibitory compounds as separate molecules for use in the treatment of a subject having an immune disease selected from the group consisting of autoimmune disease, allergic disease and graft rejection or being in need of prevention thereof, wherein said vector is administered to said subject.

Also disclosed herein is the use of a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of a self-antigen, an allergen, an alloantigen or a xenoantigen; and
(b) one or more further nucleic acid sequences encoding one or more immunoinhibitory compounds,
wherein the vector allows for the co-expression of the first polypeptide and the one or more immunoinhibitory compounds as separate molecules for the manufacture of a medicament for use in the treatment of a subject having an immune disease selected from the group consisting of autoimmune disease, allergic disease and graft rejection or being in need of prevention thereof, wherein said medicament is administered to said subject.

Also disclosed herein is the use of a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of a self-antigen, an allergen, an alloantigen or a xenoantigen; and
(b) one or more further nucleic acid sequences encoding one or more immunoinhibitory compounds,
wherein the vector allows for the co-expression of the first polypeptide and the one or more immunoinhibitory compounds as separate molecules, for treating a subject having an immune disease selected from the group consisting of autoimmune disease, allergic disease and graft rejection or being in need of prevention thereof.

Also disclosed herein is a vector comprising:
(a) a first nucleic acid sequence encoding a first polypeptide, wherein the first polypeptide comprises a targeting unit that targetes antigen-presenting cells, a multimerization unit, such as a dimerization unit, and an antigenic unit, wherein the antigenic unit comprises one or more T cell epitopes of a self-antigen, an allergen, an alloantigen or a xenoantigen; and
(b) one or more further nucleic acid sequences encoding one or more immunoinhibitory compounds,
wherein the vector allows for the co-expression of the first polypeptide and the one or more immunoinhibitory compounds as separate molecules, when used in the therapeutic or prophylactic treatment of an immune disease selected from the group consisting of autoimmune disease, allergic disease and graft rejection.

Also disclosed herein is the use of a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of a self-antigen, an allergen, an alloantigen or a xenoantigen; and
(b) one or more further nucleic acid sequences encoding one or more immunoinhibitory compounds,
wherein the vector allows for the co-expression of the first polypeptide and the one or more immunoinhibitory compounds as separate molecules for the therapeutic or prophylactic treatment of an immune disease selected from the group consisting of autoimmune disease, allergic disease and graft rejection.

Also disclosed herein is a medicament for the treatment or prevention of an immune disease selected from the group consisting of autoimmune disease, allergic disease and graft rejection in a subject having said disease or being in need of prevention of said disease by administering to the subject a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of a self-antigen, an allergen, an alloantigen or a xenoantigen; and
(b) one or more further nucleic acid sequences encoding one or more immunoinhibitory compounds,
wherein the vector allows for the co-expression of the first polypeptide and the one or more immunoinhibitory compounds as separate molecules.

### EXAMPLES

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### Example 1

DNA vectors were designed, and produced, comprising nucleotide sequences encoding murine myelin oligodendrocyte glycoprotein (MOG) 27-63 and further encoding the following elements/units:

TABLE 4

| Elements/ | VB5049* | VB5052* | VB5051* |
|---|---|---|---|
| units | SEQ ID NO: 3 | SEQ ID NO: 4 | SEQ ID NO: 5 |
| Signal | Murine Ig VH | Natural leader | Murine Ig VH |
| peptide | signal peptide | sequence human | signal peptide |
| | SEQ ID NO: 2 | CCL3L1 | SEQ ID NO: 2 |
| | | SEQ ID NO: 13 | |
| Targeting | scFv with | Human CCL3L1 | NA |
| unit | specificity for | SEQ ID NO: 14 | |
| | murine CD205 | | |
| | SEQ ID NO: 15 | | |
| Dimerization | Hinge-region | Hinge-region 1 | NA |
| unit | 1 from | from human IgG3 | |
| | human IgG3 | Hinge-region 4 | |
| | Hinge-region 4 | from human | |
| | from human | IgG3 | |
| | IgG3 Glycine- | Glycine-serine- | |
| | serine-linker | linker | |
| | Human IgG3 | Human IgG3 | |
| | CH3 domain | CH3 domain | |
| | SEQ ID NO: 1 | SEQ ID NO: 1 | |
| Unit linker | Glycine-leucine | Glycine-leucine | NA |
| | linker | linker | |
| | Amino acids | Amino acids | |
| | 410-414 of | 410-414 of | |
| | SEQ ID NO: 3 | SEQ ID NO: 3 | |
| Antigenic | Murine myelin oligodendrocyte glycoprotein | | |
| unit | (MOG) 27-63* SEQ ID NO: 16 | | |
| Co- | T2A peptide | NA | NA |
| expression | SEQ ID NO: 6 | | |
| element | | | |
| Signal peptide | Natural leader | NA | NA |
| for immuno- | sequence | | |
| inhibitory | murine IL-10 | | |
| compound | SEQ ID NO: 17 | | |
| Immuno- | Murine IL-10 | NA | NA |
| inhibitory | SEQ ID NO: 18 | | |
| compound | | | |

*The MOG (27-63) sequence was obtained from Krienke et al., Science 371, 145-153, 2021.

Myelin oligodendrocyte glycoprotein (MOG) is a protein expressed in the central nervous system. The MOG (27-63) sequence comprised in the antigenic unit of the above-described DNA vectors comprises the immunodominant 35-55 T cell epitope of MOG, MOG (35-55), which is a primary target for both cellular and humoral immune responses during multiple sclerosis. MOG (35-55)-induced experimental autoimmune encephalomyelitis (EAE) is the most commonly used animal model of multiple sclerosis.

In the following, “m”, “murine” and “mouse” are used interchangeably, and “h” and human are used interchangeably.

The DNA vector VB5049* (SEQ ID NO: 3), a vector according to the invention, encodes a first polypeptide comprising the targeting unit, dimerization unit, unit linker and antigenic unit comprising as stated in Table 4 above and IL-10 as immunoinhibitory compound. Due to the presence of the co-expression element T2A, the first polypeptide and the immunoinhibitory compound are expressed as separate molecules.

The DNA vector VB5052* (SEQ ID NO: 4) encodes a first polypeptide comprising a human macrophage inflammatory protein alpha variant targeting unit (hCCL3L1, also called LD78β or hMIP1α), which is known to target APCs in an immunogenic manner, i.e. first polypeptides/dimeric proteins comprising such a targeting unit will induce a pro-inflammatory immune response in subjects to which they are administered (see for instance WO 2011/161244 A1). VB5052* is a “pro-inflammatory version” of VB5049* and used as a comparison to the constructs of the invention.

The DNA vector VB5051* (SEQ ID NO: 5) encodes only the antigenic unit, i.e., MOG (27-63); a single protein/peptide. VB5051* is used as a comparison to the constructs of the invention.

### Production of DNA Vectors

All DNA vectors in this Example section were produced by ordering sequences as stated in the tables in this Example section from Genscript Biotech B.V., Netherlands, and cloning them into the expression vector pALD-CV77, a DNA plasmid.

### Example 2

The purpose of this study was to characterize protein expression and secretion of proteins encoded by the DNA vectors VB5049* and VB5052* post transient transfection of mammalian cells.

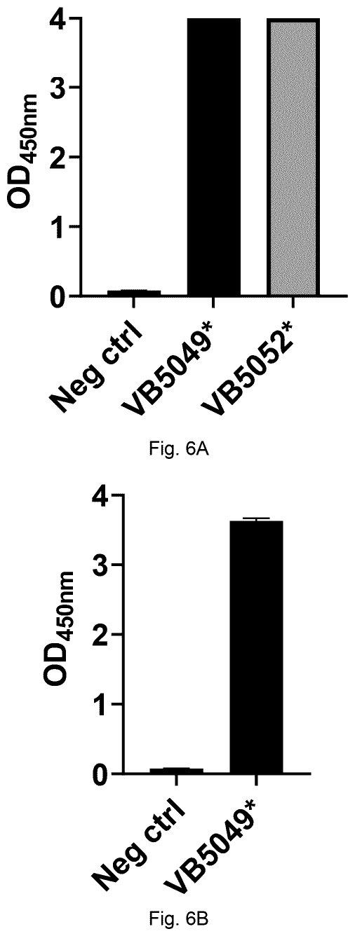

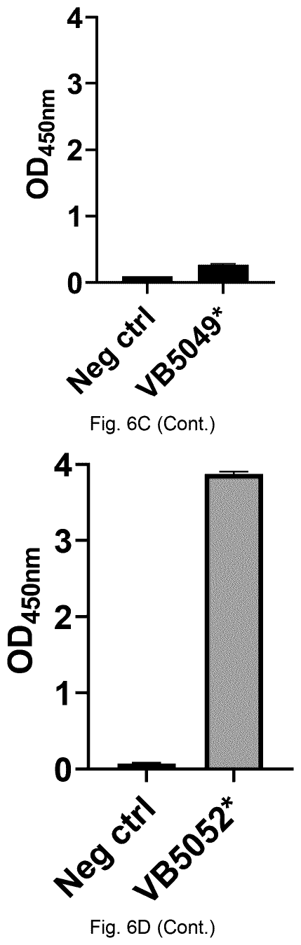

HEK293 cells were obtained from ATCC and transiently transfected with VB5049* or VB5052* DNA vectors. Briefly, 2×10⁵ cells/well were plated in 24-well tissue culture plates with 10% FBS growth medium and transfected with 1 μg of the respective DNA vector using Lipofectamine® 2000 reagent (Invitrogen, Thermo Fischer Scientific). The transfected cells were then maintained for 5 days at 37° ° C. with 5% CO₂, then the cell supernatant was collected for characterization of the secreted proteins encoded by VB5049* or VB5052* by sandwich ELISA of the supernatant using antibodies against hIgG CH3 domain (detection antibody, 100 μl/well, 0.1 μg/ml mouse anti-human IgG Fc secondary antibody, biotin (05-4240, Invitrogen)) and MOG (capture antibody, 100 μl/well, 0.25 μg/ml mouse anti-MOG antibody (NYRMOG, sc-73330, Santa Cruz Biotechnology)). The results are shown in FIG. 6A.

The secretion of IL-10 encoded by VB5049* was measured by a sandwich ELISA using antibodies against murine IL-10 (capture antibody, 100 μl/well, 0.4 μg/ml rat anti-mouse IL-10 antibody (MAB417, R&D Systems), detection antibody, 100 μl/well, 0.2 μg/ml goat anti-mouse IL-10 biotinylated antibody (BAF417, R&D Systems)). The results are shown in FIG. 6B.

The secretion of the first polypeptide and IL-10 as two separate proteins from VB5049* was shown by sandwich ELISA using antibodies against murine MOG (capture antibody, 100 μl/well, 0.25 μg/ml mouse anti-MOG antibody (NYRMOG, sc-73330, Santa Cruz Biotechnology) and murine IL-10 (detection antibody, 100 μl/well, 0.2 μg/ml goat anti-mouse IL-10 biotinylated antibody (BAF417, R&D Systems). The results are shown in FIG. 6C.

The secretion of the first polypeptide encoded by VB5052* was further shown by sandwich ELISA using antibodies against hIgG CH3 domain (capture antibody, 100 μl/well, 1 μg/ml, mouse anti-human IgG (CH3 domain), 153272, Biorad) and hCCL3L1 (detection antibody, 100 μl/well, 0.2 μg/ml, goat anti-human CCL3L1 biotin antibody). Results are shown in FIG. 6D.

As evident from FIG. 6A (capture antibody: anti-MOG antibody, detection antibody: anti-hIgG CH3 domain antibody) and 6D (capture antibody: anti-hIgG CH3 domain, detection antibody: anti-human CCL3L1) both first polypeptides encoded by VB5049* and VB5052*, respectively, were secreted at high levels.

FIG. 6B shows the level of IL-10 secretion in undiluted supernatant. By comparing a dilution curve of the supernatant with a dilution curve of recombinant IL-10, the secretion level was estimated to be 2.25 μg/ml in the supernatant.

FIG. 6C shows the ELISA results obtained with anti-MOG antibody as a capture antibody and anti-murine IL-10 antibody as the detection antibody. As expected, VB5049* gave a very weak signal, confirming that the first polypeptide and IL-10 are expressed as two separate molecules due to the presence of the T2A peptide.

The results depicted in FIGS. 6A-D show that the first polypeptide/dimeric protein (comprising the targeting unit, dimerization unit and antigenic unit) encoded by the DNA vectors VB5049* and VB5052* are secreted from transfected cells and that, for VB5049*, IL-10 is also expressed and secreted as a separate, protein.

To further characterize the expressed proteins, western blot (WB) analysis was performed. Briefly, Expi293F cells (3×10⁶ cells/ml, 1.6 ml) were seeded in a 6-well culture plate. The cells were transfected with 1 μg/ml plasmid DNA using ExpiFectamine 293 Reagent (Thermo Fisher Sci.), and the plates were incubated on an orbital shaker (19 mm diameter, 125 rpm) in a humidified CO₂ cell incubator (8% CO₂, 37° C.). After 18 h of incubation, ExpiFectamine 293 Transfection Enhancer (Thermo Fisher Sci.) was added to each well. The plates were incubated for another 28 h before the supernatant was harvested.

The samples were prepared by mixing 105 μl supernatant from transfected Expi293F cells with 37.5 μl 4× Laemmli sample buffer (Bio-Rad) with 7.5 μl DTT (Thermo Fisher Sci.) or 7.5 μl ultrapure water for reducing and non-reducing conditions, respectively. The samples (reduced or non-reduced) were heated at 70° C. for 10 minutes before 10 μl was added to 4%-20% Criterion TGX Stain-Free precast gels (Bio-Rad). SDS-PAGE was performed in 1× Tris/Glycine/SDS running buffer (Bio-Rad) with a Precision Plus Protein All Blue Prestained protein standard (Bio-Rad). Proteins were transferred from the gel onto EtOH activated low fluorescence (LF) 0.45 μm PVDF membranes (Bio-Rad) by using the Tran-Blot Turbo semi-dry transfer system (Bio-Rad). PVDF membranes were blocked in EveryBlot buffer (Bio-Rad) for 5 min and probed with mouse anti-MOG (sc-73330, Santa Cruz Biotechnology) and rat anti-mouse IL-10 antibody (MAB417, R&D systems) to detect the first polypeptide/dimeric protein and IL-10, respectively. The membranes were incubated with fluorochrome-conjugated secondary antibodies for 1 h at room temperature, and then washed and dried. Images were acquired by using a ChemiDoc™ MP Imaging System (setting Dylight 488 and 800, Auto Optimal).

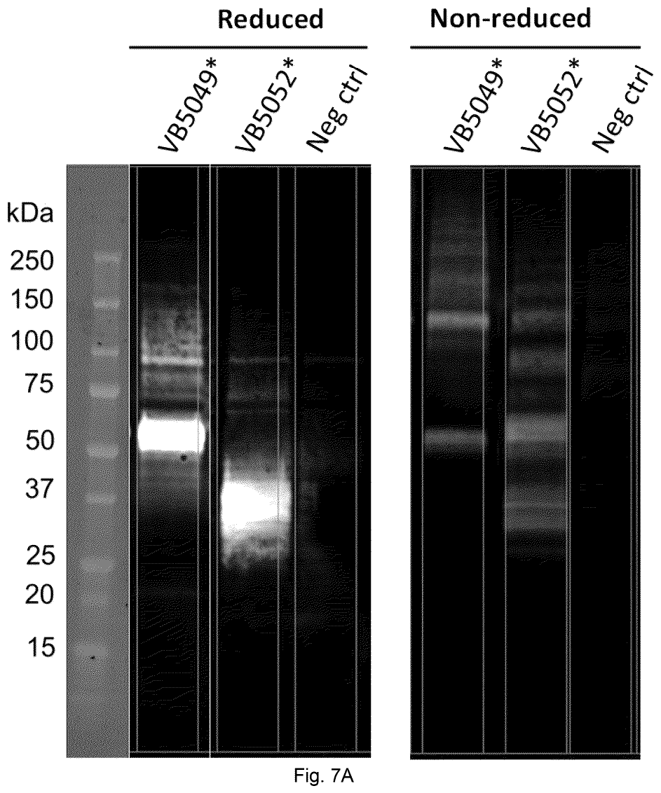

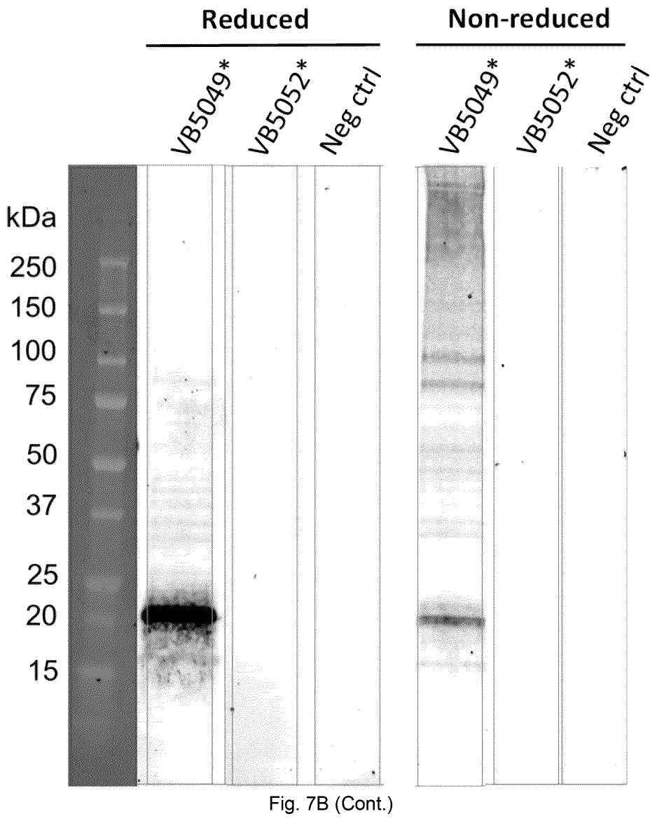

FIGS. 7A and 7B show the successful expression and secretion of the first polypeptide/dimeric proteins encoded by VB5049* and VB5052* and IL-10 encoded by VB5049*.

FIG. 7A shows that proteins expressed by VB5052* and VB5049*, secreted from transfected cells, and run on an SDS-PAGE under reducing and non-reducing conditions could be detected. The membranes were probed with mouse anti-murine MOG. Under reducing conditions the first polypeptide encoded by VB5052* is detected as a monomer (31 kDa), while under non-reducing conditions, it was detected both as a monomer (31 kDa, first polypeptide) and a dimer (61 kDa, dimeric protein). Under reducing conditions, the first polypeptide encoded by VB5049* was detected as a monomer (51 kDa), while under non-reducing conditions, it was detected as both a monomer (51 kDa, first polypeptide) and a dimer (101 kDa, dimeric protein).

FIG. 7B shows the detection of IL-10 (18 kDa). There was no detection of a protein of 71 kDa, confirming that IL-10 was expressed from VB5049* as a separate protein.

Taken together, the ELISA and western blot data demonstrate that intact first polypeptides/dimeric proteins can be co-expressed from a DNA vector together with mIL 10 by using a T2A peptide as co-expression element.

### Example 3

Tolerance-inducing ability of VB5049* was determined by calculating the ratio of IL-10/IFN-γ from measuring IL-10 (an anti-inflammatory cytokine associated with immune tolerance) and IFN-γ (a marker for inducing an inflammatory immune response). This ratio illustrates to which extend the immune response induced by VB5049* is skewed towards a tolerogenic profile.

The following study design was applied:

Female, 6-week-old C57BL/6 mice were obtained from Janvier Labs (France). All animals were housed in the animal facility at the Radium Hospital (Oslo, Norway). All animal protocols were approved by the Norwegian Food Safety Authority (Oslo, Norway). 5 mice/group were used for the testing of VB5049*, VB5052* and VB5051*, whereas 2 mice/group were used for the negative control (PBS only). VB5052* is a pro-inflammatory version of VB5049* and was included as a comparison to VB5049*. It is expected that the pro-inflammatory version of VB5049*, VB5052*, induces IFN-γ production. VB5051* was included as a comparison to VB5049*.

One dose of 50 μg of the respective DNA vector dissolved in sterile PBS was administered by intramuscular needle injection to each tibialis interior (2×25 μl, 1000 μg/ml), followed by electroporation with AgilePulse in vivo electroporation system (BTX, USA).

The spleens were collected 7 days after administration and mashed in a cell strainer to obtain a single cell suspension. The red blood cells were lysed using ammonium-chloride-potassium (ACK) lysing buffer. After washing, the splenocytes were counted using NucleoCounter NC-202 (ChemoMetec, Denmark), re-suspended to a final concentration of 6×10⁶ cells/ml and seeded as 6×10⁵ cells/well in a 96-well IFN-γ/IL-10 dual color FluoroSpot plate. Splenocytes were then re-stimulated for 44 h with 16.67 μg/ml MOG (35-55) peptide before tested for IFN-γ and IL-10 cytokine production in a dual color FluoroSpot according to the manufacturer's protocol (Mabtech AB, Sweden). Spot-forming cells were measured in an IRIS FluoroSpot and ELISpot plate reader (Mabtech AB) and analyzed using the Apex software (Mabtech AB). Results are shown as the mean number of triplicates of IL-10+ or IFN-γ+ spots/106 splenocytes.

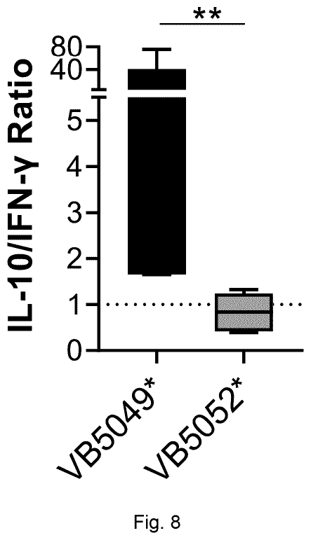

As can be seen from FIG. 8, the ratio of IL-10/IFN-γ obtained with VB5049* is high, indicating that VB5049* induces significantly higher levels of the immunoinhibitory cytokine IL-10 than the inflammatory cytokine IFN-γ. This is not the case for VB5052* which showed an IL-10/IFN-γ ratio of about 1, indicating that both cytokines are produced at equivalent levels after re-stimulation with MOG (35-55) peptide.

The total splenocytes obtained from mice administered with the DNA vectors were also analyzed with multicolor flow cytometry. Briefly, cells were re-stimulated with MOG (35-55) peptide for 16 hours and then harvested and counted. 2×10⁶ cells per sample were used for flow cytometry analysis. Cells were first stained with fixable viability dye (eFluor780) for 10 min at room temperature in the dark. Following this, the cells were washed in 1×PBS (centrifuged (400 g/10 min/4° C.)) and incubated with a surface staining antibody (Ab) mix (anti-CD3 BUV395, anti-CD4 BV785, and anti-CD8) at 4° C. in the dark for 30 min. Following incubation, the cells were washed, and cell pellets were re-suspended in flow buffer (PBS, 10% FBS and 2 mM EDTA). The cells were then fixed and permeabilized (eBioscience™ Foxp3/Transcription Factor Staining Buffer Set) for 45-60 min at 4ºC in the dark. Subsequently, the cells were washed in 1× permeabilization buffer, centrifuged as previously described, and cell pellets were re-suspended in intracellular staining Ab mix (anti-IFN-γ APC, anti-IL-17 Alexa fluor 488, BV421, anti-Foxp3 PE). The cells were incubated with the intracellular staining Ab mix at 4° C. in the dark for 30 min. The cells were subsequently washed and re-suspended in flow buffer until flow cytometry was carried out on a BD Symphony A5 flow cytometer. Compensation was set up using single-stained Ultra comp eBeads for fluorochrome conjugated Abs, and ArC reactive beads for fixable viability dye. Flow cytometry files were analyzed using FlowJo software.

Both IFN-γ and IL-17 are pro-inflammatory cytokines contributing to the pathogenesis of chronic inflammatory and autoimmune diseases, including experimental autoimmune encephalomyelitis (EAE) and multiple sclerosis. Thus, tolerance-inducing construct must reliably induce tolerance without inadvertently sensitizing auto-antigen immune responses, e.g. by inducing pro-inflammatory cytokines that may exacerbate autoimmunity.

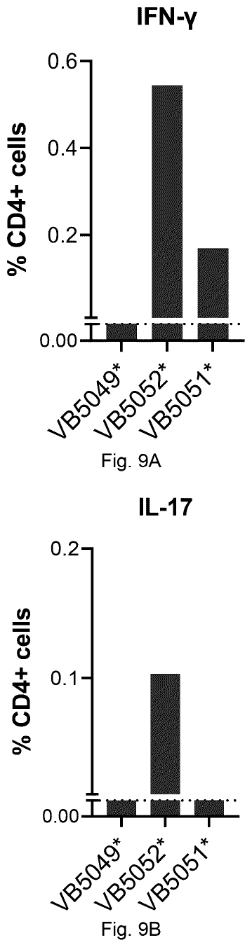

FIGS. 9A and 9B show that the lack of IFN-γ production upon VB5049* administration, as observed in the FluoroSpot assay, was confirmed with flow cytometry. Moreover, the flow cytometry also demonstrated that VB5049* did not induce IL-17 production. Conversely, administration of the pro-inflammatory VB5052* induced production of IFN-γ and IL-17, as may be expected. VB5051* administration induced some IFN-γ production in the splenocytes, but very low levels of IL-17.

The generation of MOG-specific regulatory T cells (Tregs), i.e. T cells that act to suppress and control MOG-specific inflammatory immune response and thereby maintaining self-tolerance, was identified by MOG-specific tetramer staining and flow cytometry (CD4+ MOG(35-55)-tet+Foxp3+ cells).

Briefly, 1 to 2×10⁶ splenocytes pooled from each group were transferred to a 96 well V bottom plate. Tetramers and antibodies were diluted in PBS with 5% FBS before use and protected from light. All steps that required cell wash were performed using PBS with 5% FBS unless otherwise stated. First, the cells were stained with either T-Select MHC Class II Tetramers specific for MOG (35-55) (10 μl per well, H-2 IAb MOG (35-55) Tetramer-PE, TS-M704-1, MBL International Corporation) or ProT2® MHC Class II Tetramers specific for MOG (38-49) (1 μg/ml, H-2 IAb-GWYRSPFSRVVH-ProT2® Tetramer PE, 2958, Proimmune) according to manufacturer instructions. Without washing the cells, FC receptors were blocked on ice for 5 min to prevent non-specific binding of flow cytometry antibodies (Ab) to the Fc receptor (0.25 μg/ml, TruStain FcX™ PLUS (anti-mouse CD16/32) Antibody, 156604, BioLegend). Without washing, the cells were stained 30 min on ice with surface Ab cocktail containing Anti-Mouse CD8 PE-Cy7 (0.25 μg/ml, Clone: 53-6.7, 100721, BD Biosciences), Anti-Mouse CD4 eFluor450 (0.25 μg/ml, Clone: GK1.5, 48-0041-82, Thermo Fischer/eBioscience) and Anti-Mouse CD25 PerCP-Cy5.5 (0.25 μg/ml, Clone: PC61, 102030, BioLegend). Cells were washed twice with PBS. Next, the cells were stained on ice for 10 min with fixable viability dye (150 μl per well, 1:8000 diluted in PBS, Fixable Viability Stain 780, 565388, BD biosciences). The cells were washed twice with PBS with 5% FBS and fixed and permeabilized using Foxp3/Transcription Factor Staining Buffer Set according to manufacturer instruction (200 μl per well, 00-5523-00, Thermo Fischer/eBioscience). The cells were washed and stained for 30 min on ice with intracellular Ab cocktail containing Anti-Mouse FOXP3 eFluor 660 (0.25 μg/ml, Clone: FJK-16s, 50-5773-82, Thermo Fischer/eBioscience) and Anti-Mouse Ki-67 Alexa Fluor 488 (0.25 μg/ml, Clone: Clone: 11F6, 151204, BioLegend). The cells were washed twice, re-suspended in 200 μl of PBS with 5% FBS and analyzed with BD FACSymphony™ A3 Cell Analyzer. The following controls were used as a guide for gating desired population using FlowJo™ v10.8 Software (BD Life Sciences): unstained control (cells were not treated with Ab) and Fluorescence Minus One (FMO) control (samples stained with all the fluorophore-labeled Abs used herein, minus the one fluorophore to be gated).

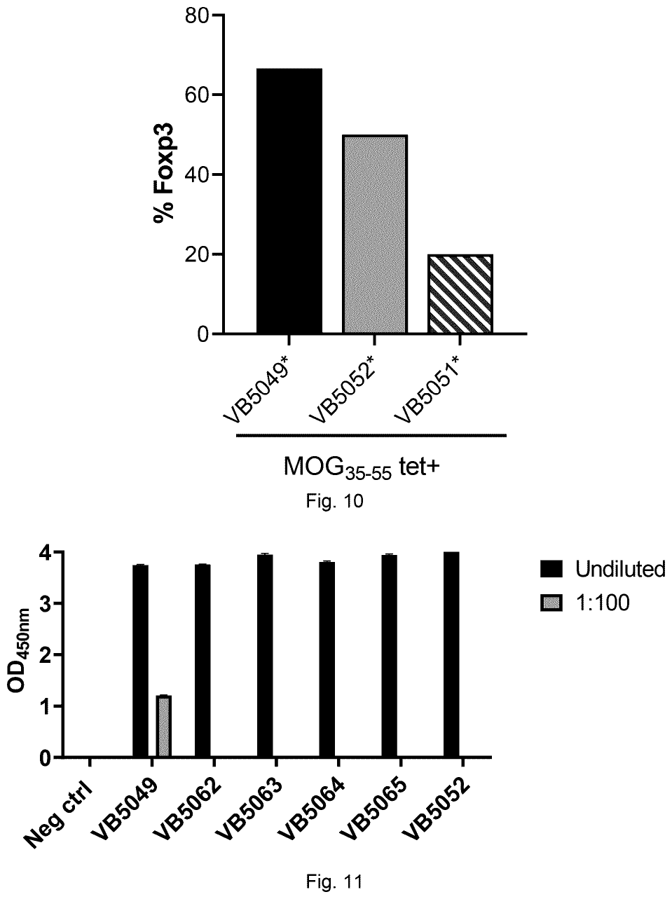

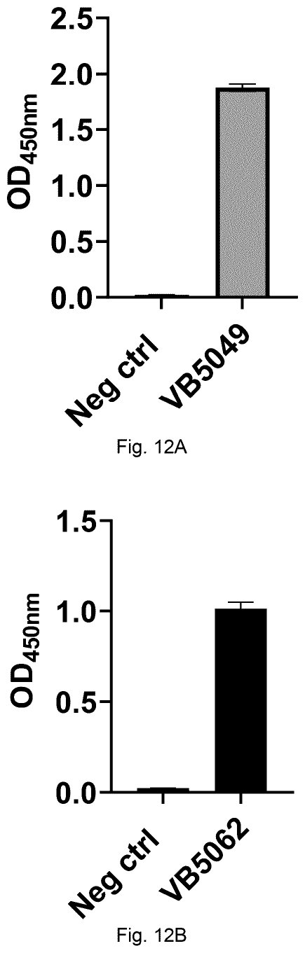

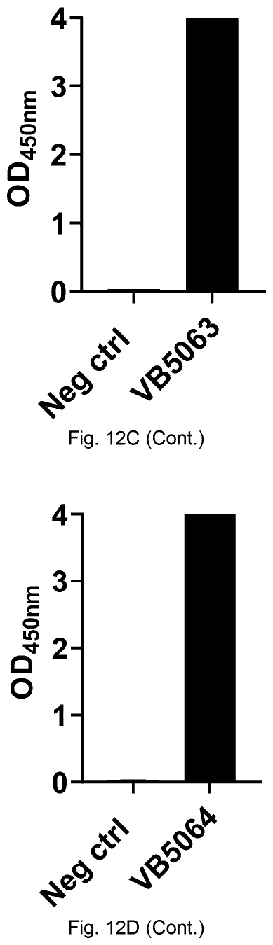

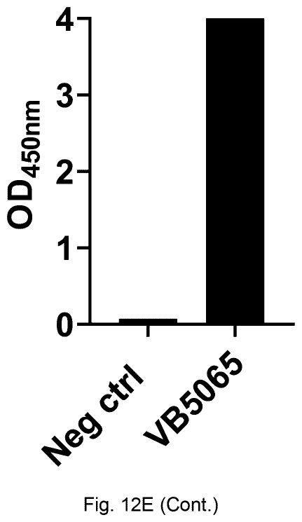

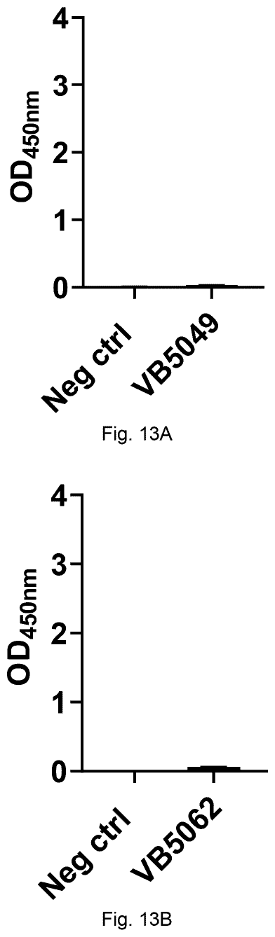

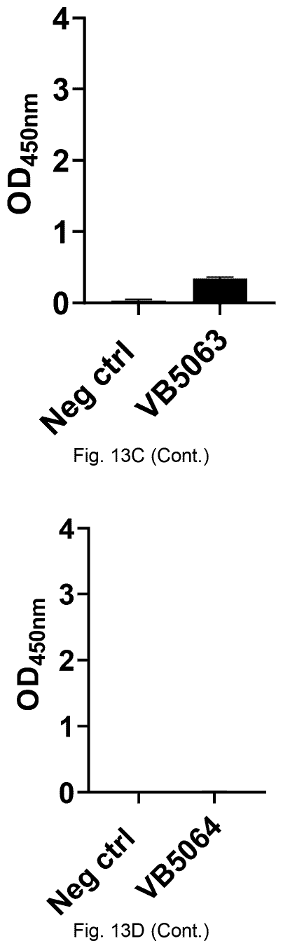

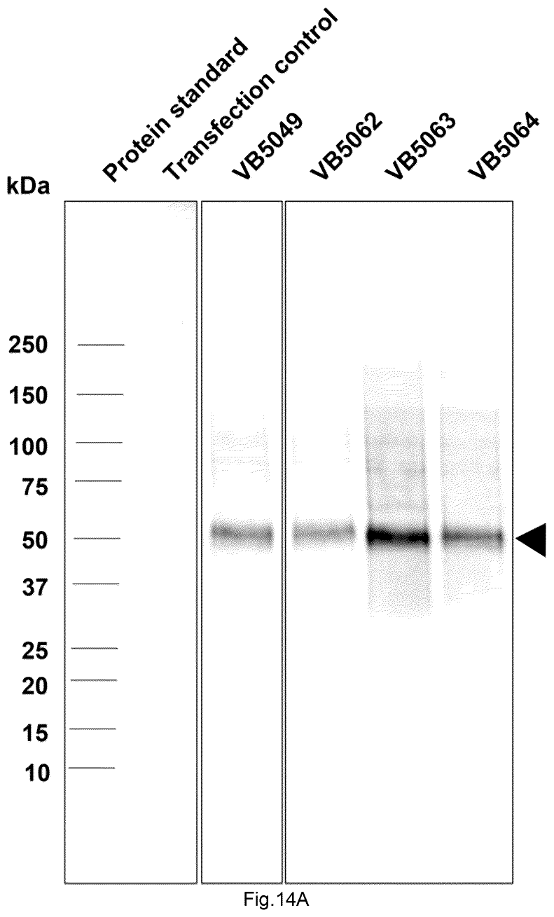

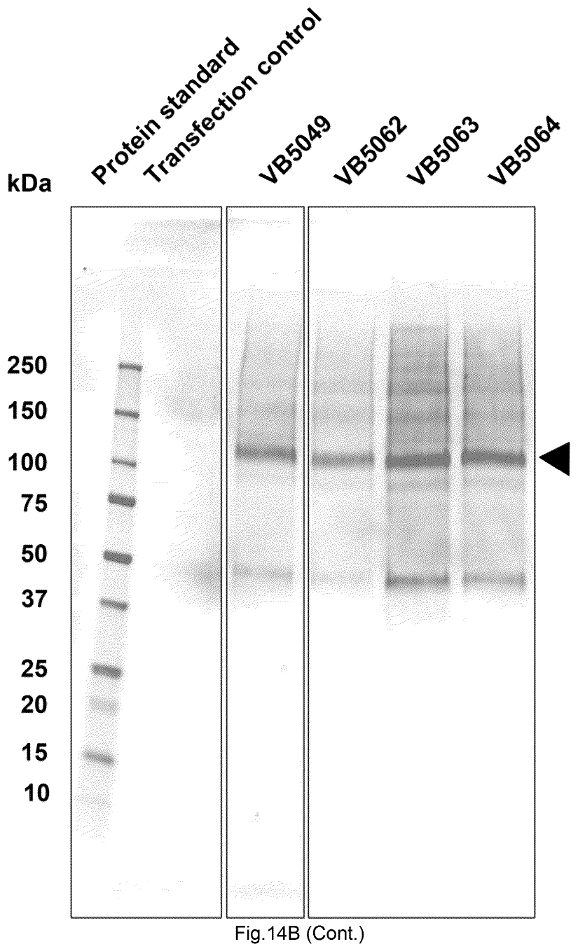

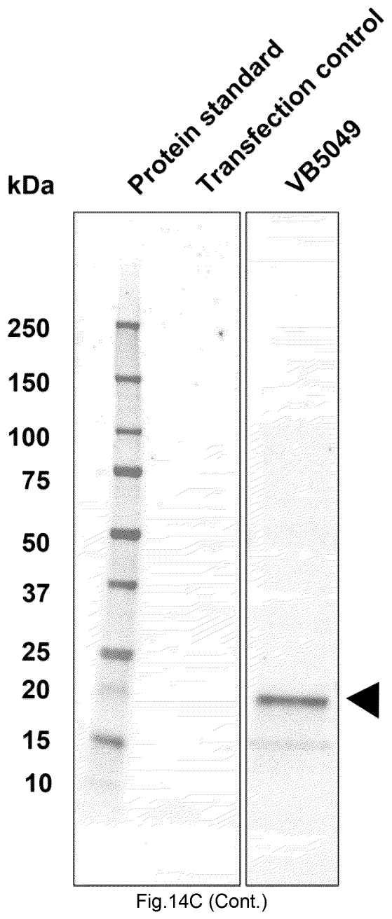

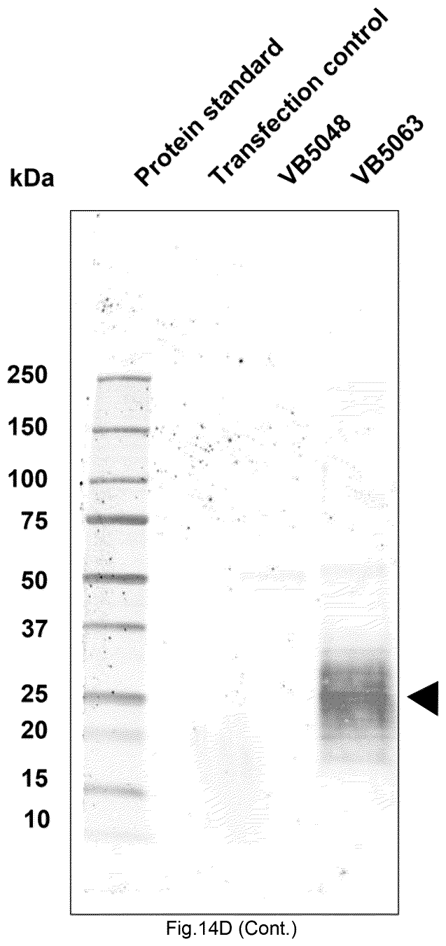

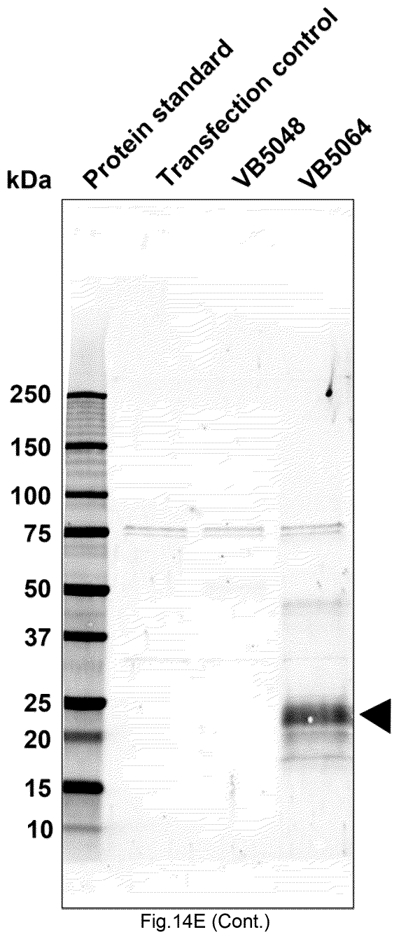

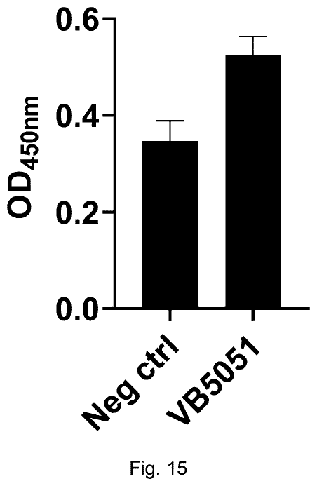

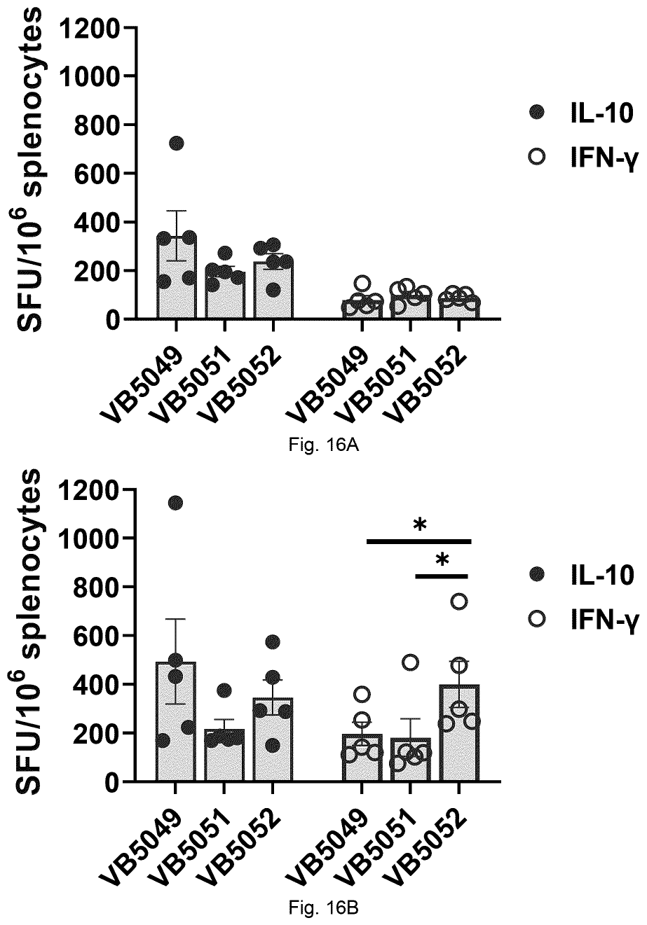

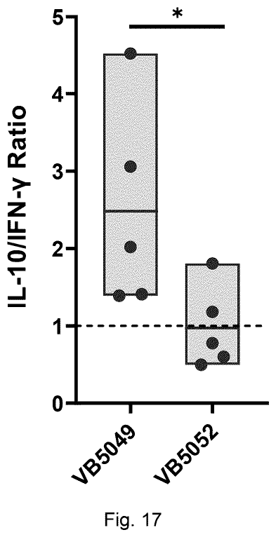

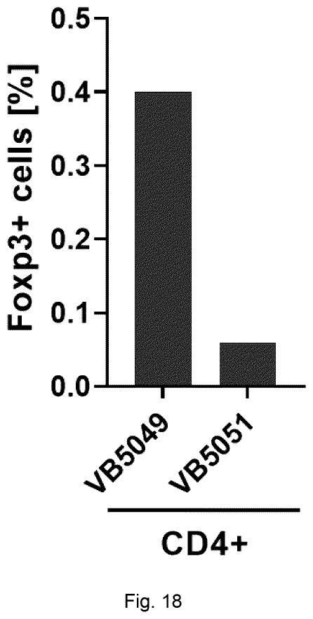

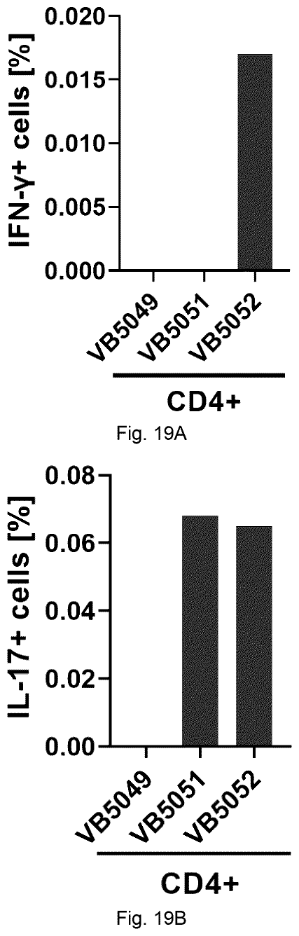

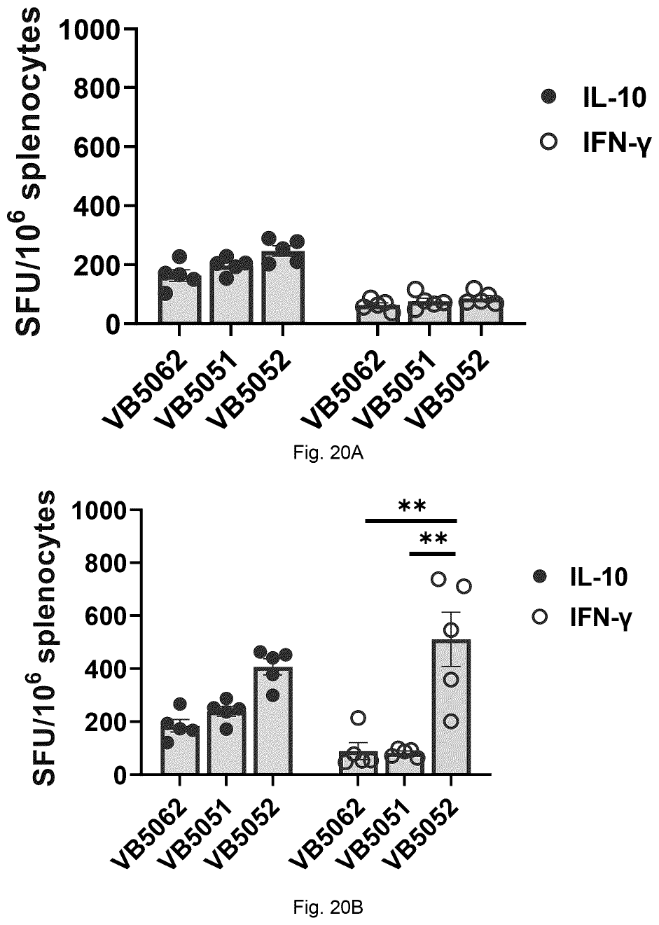

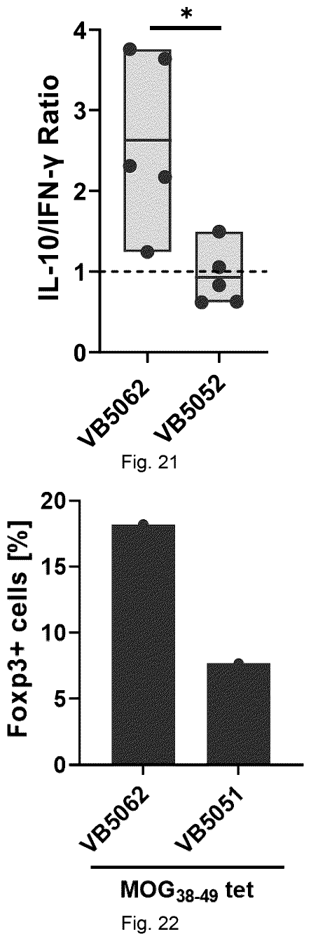

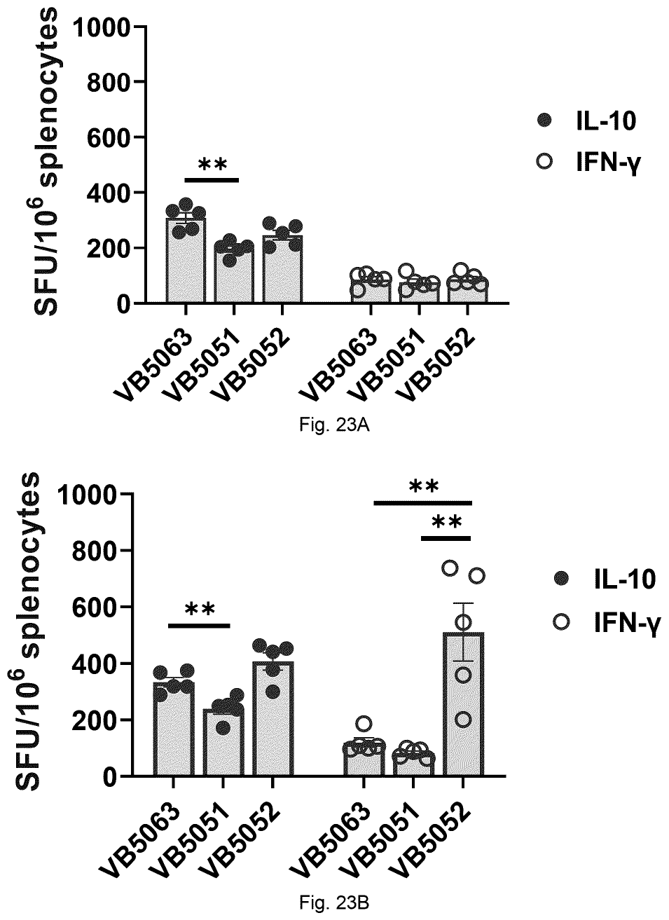

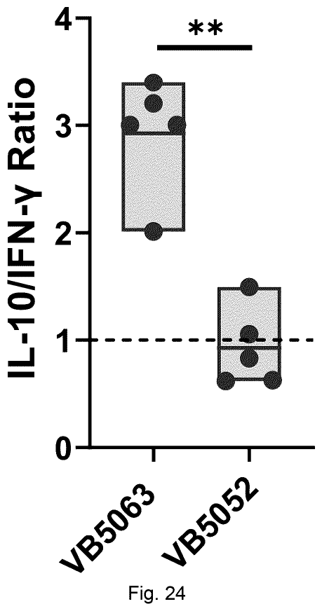

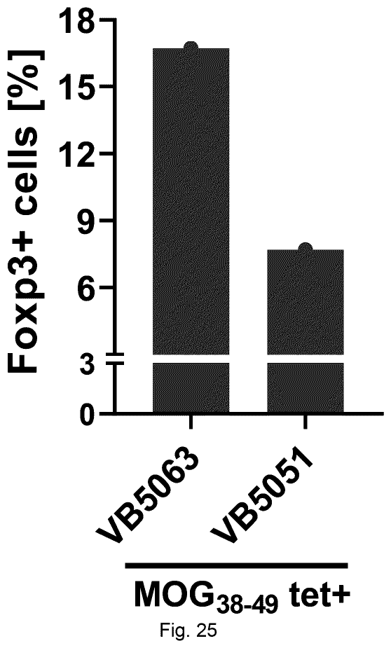

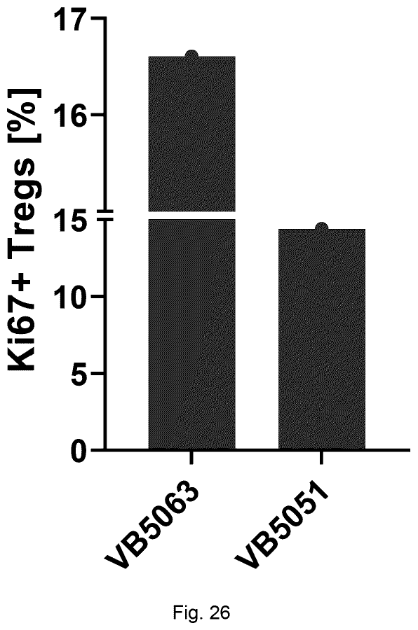

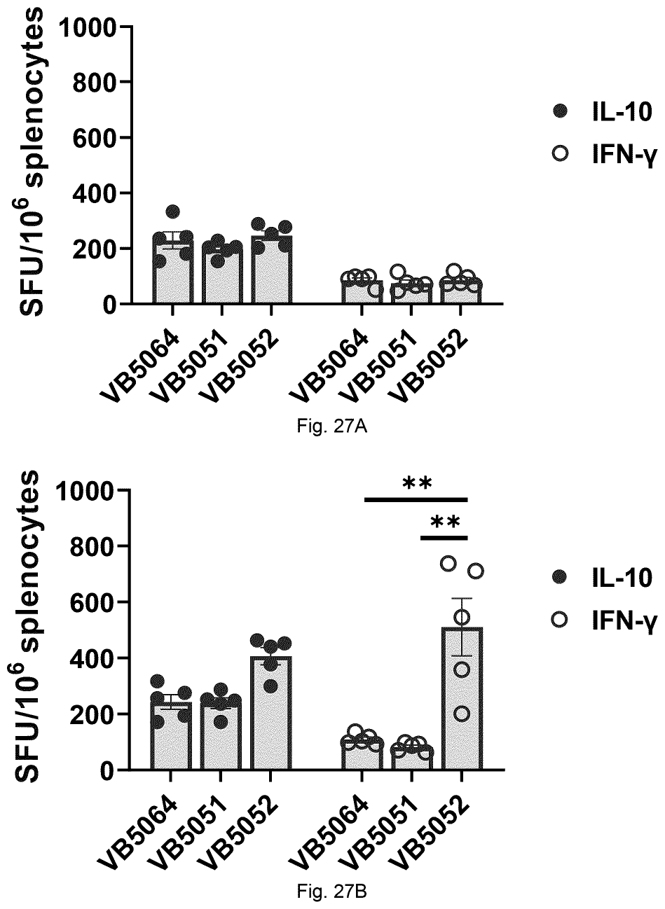

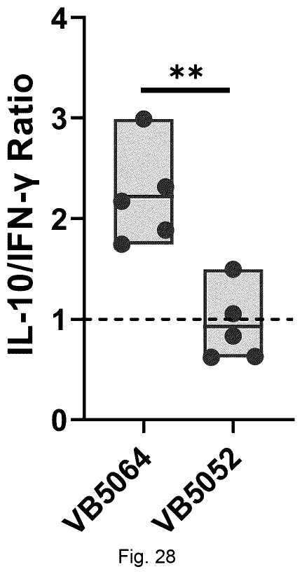

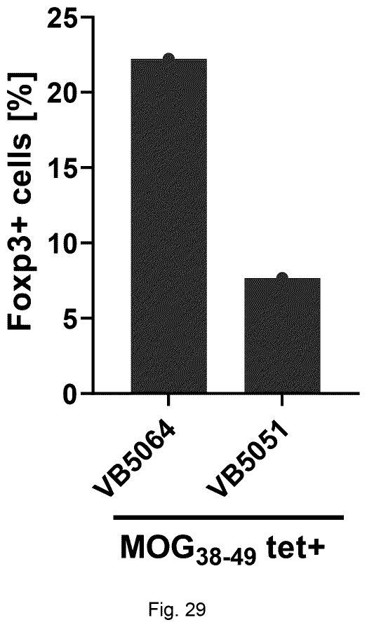

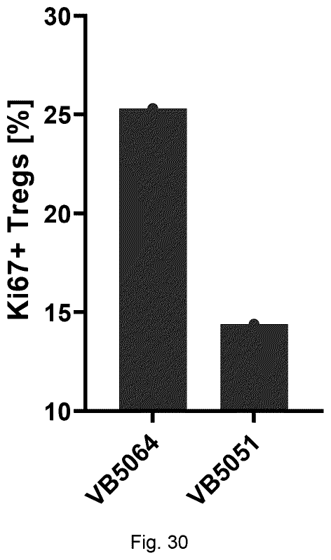

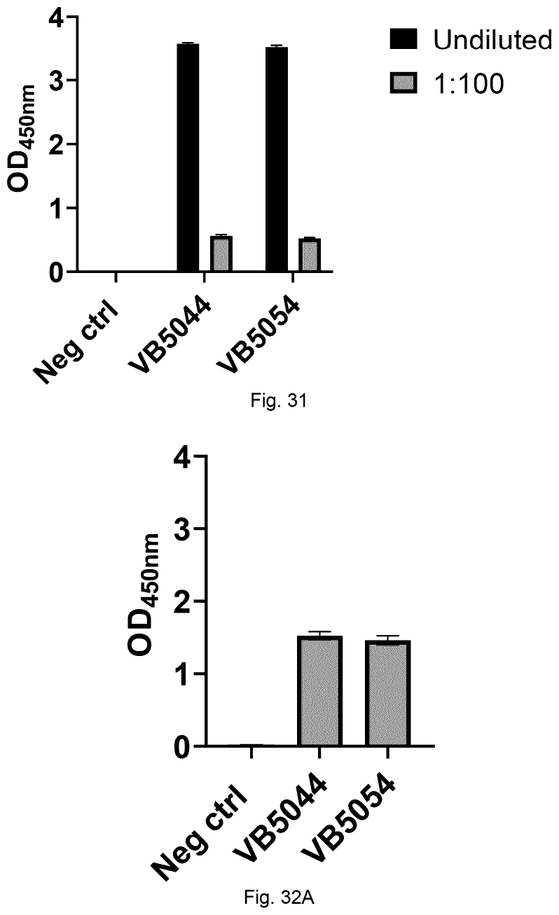

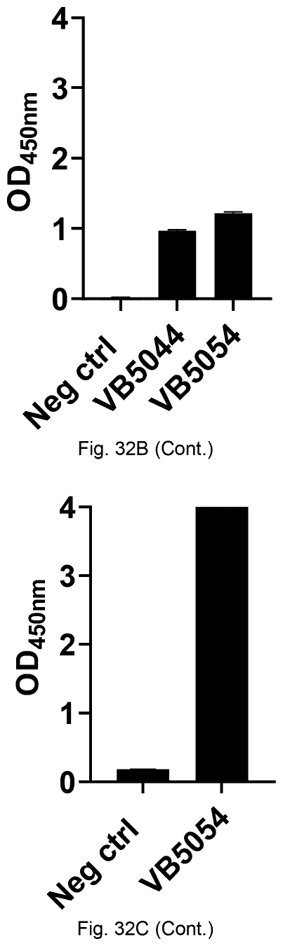

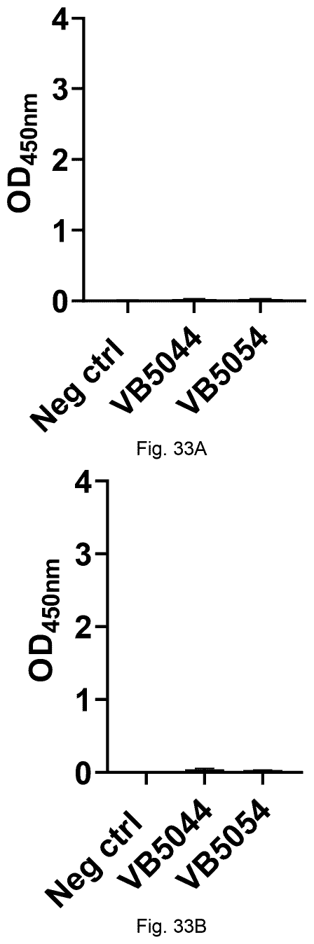

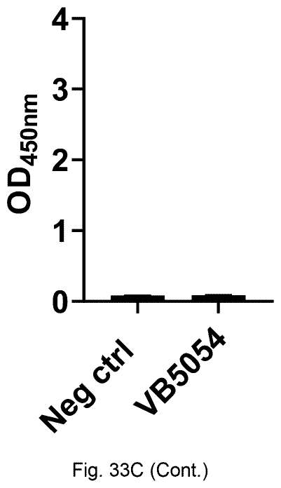

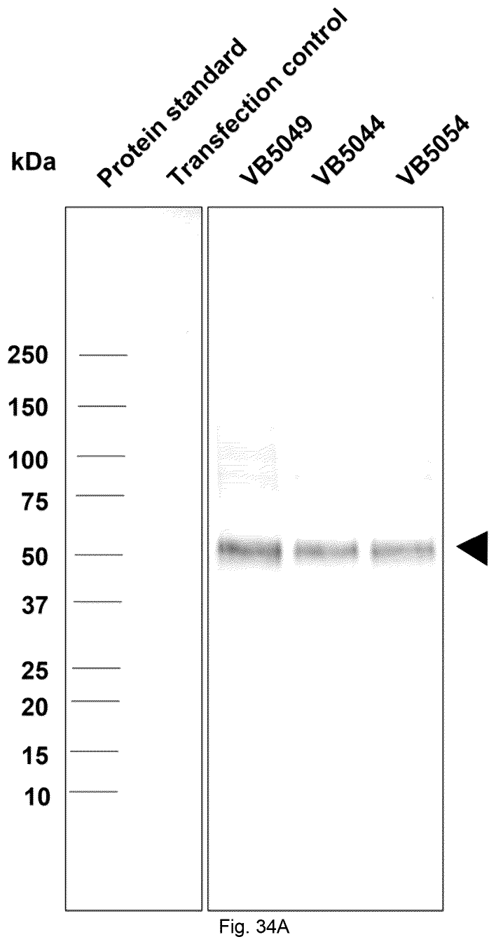

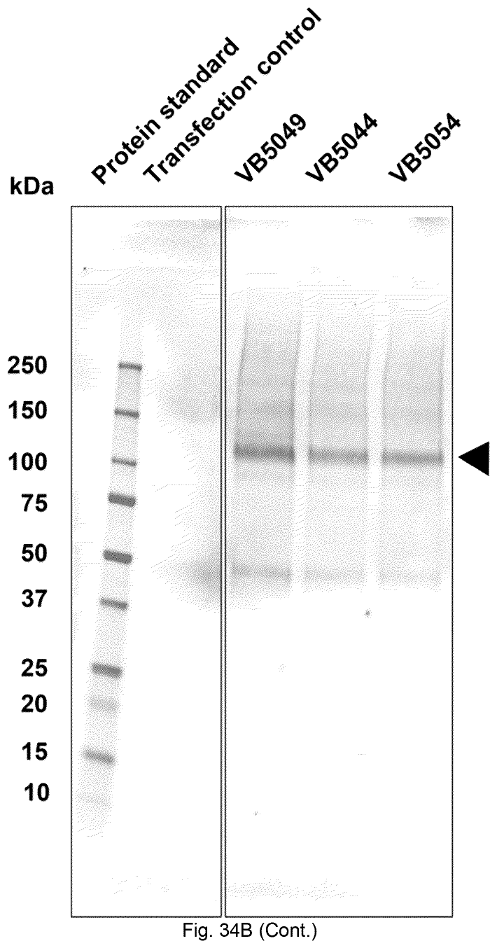

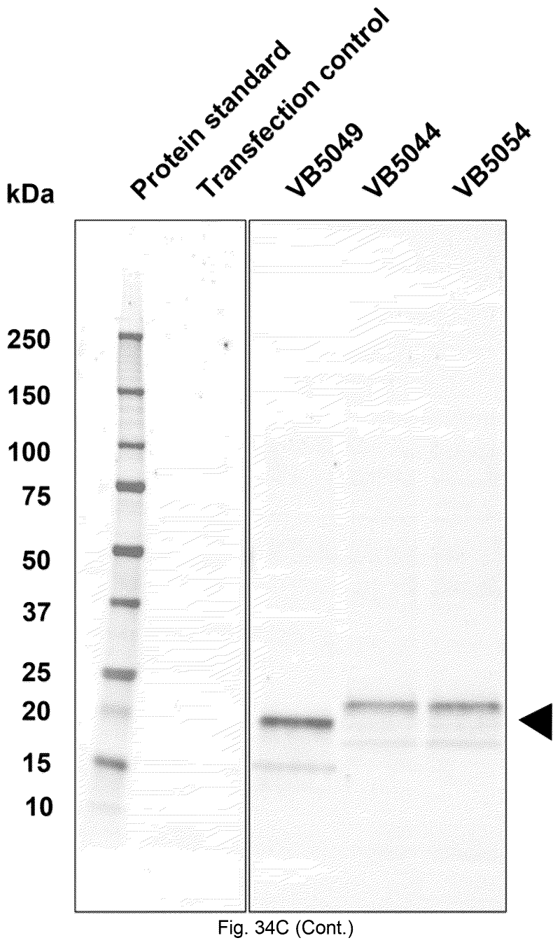

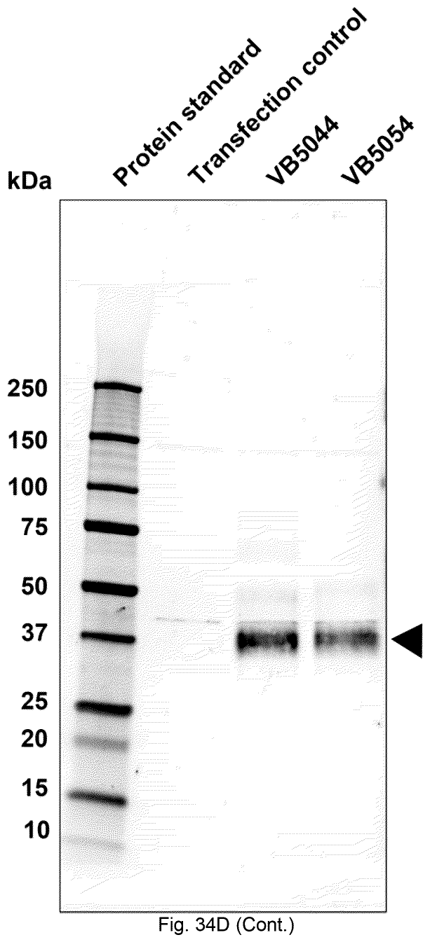

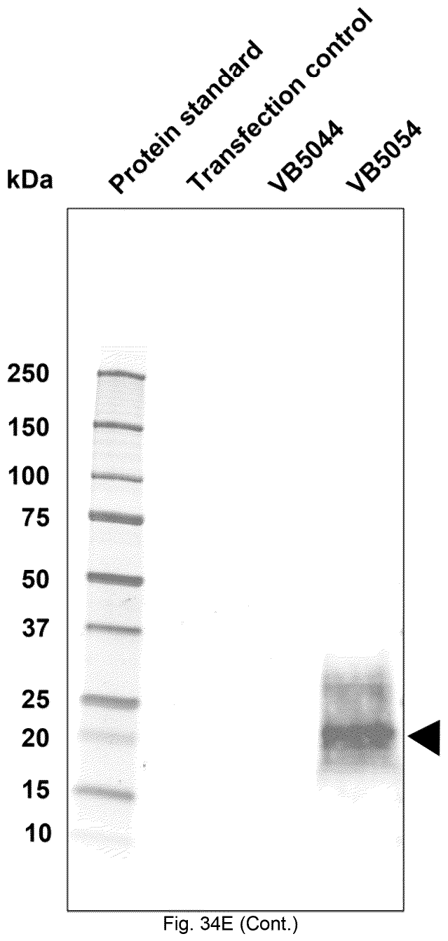

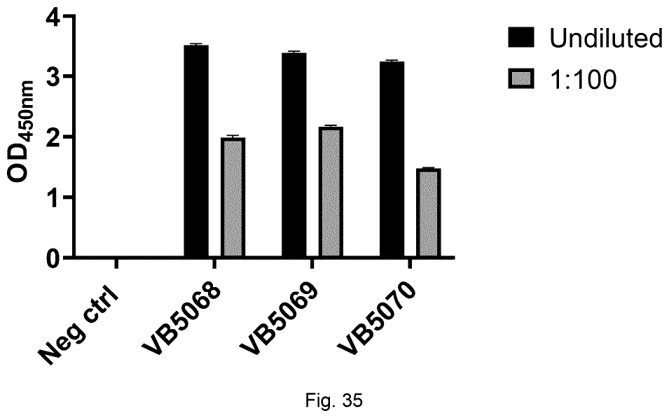

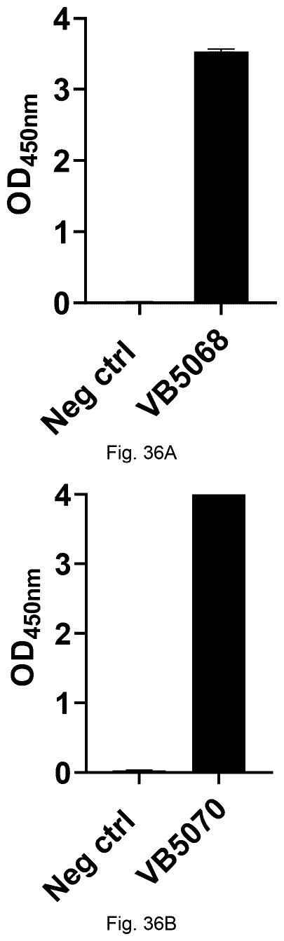

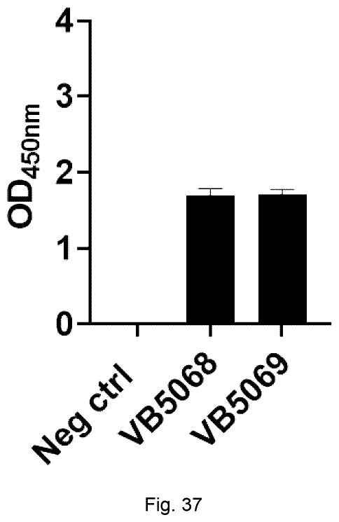

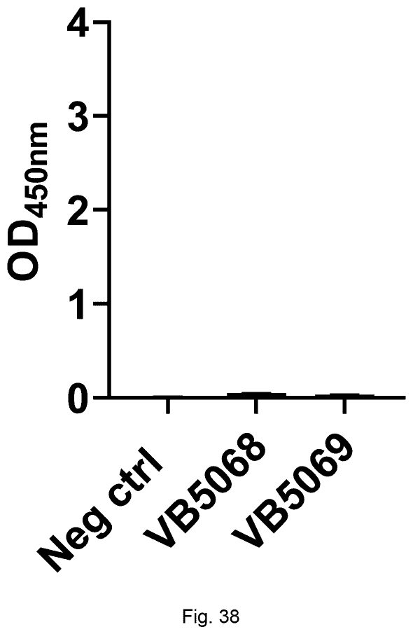

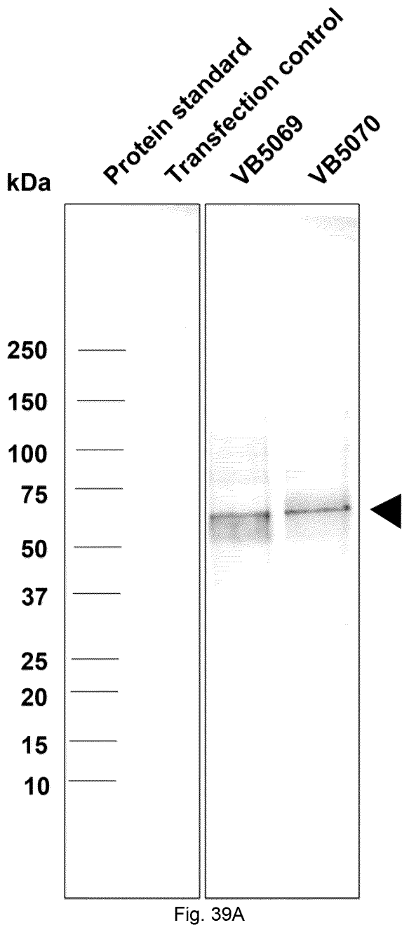

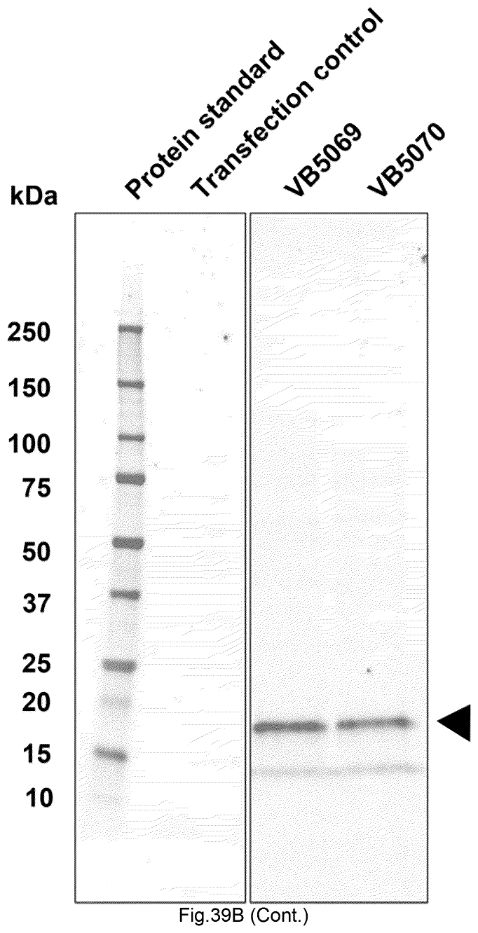

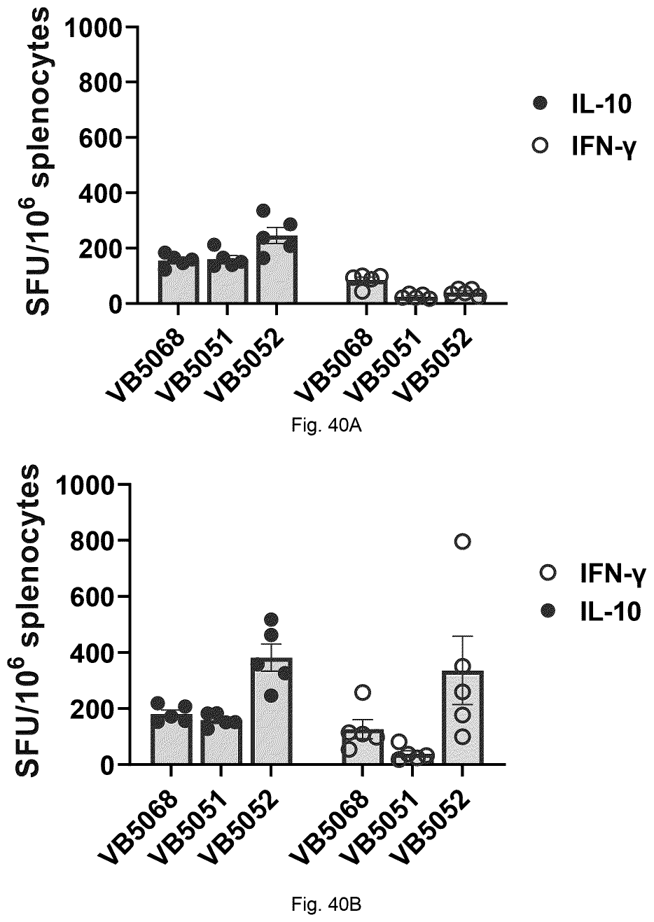

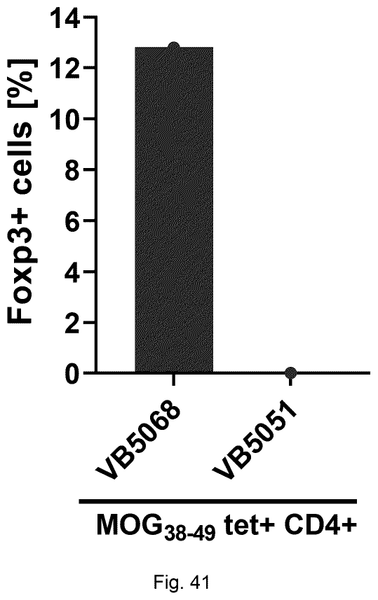

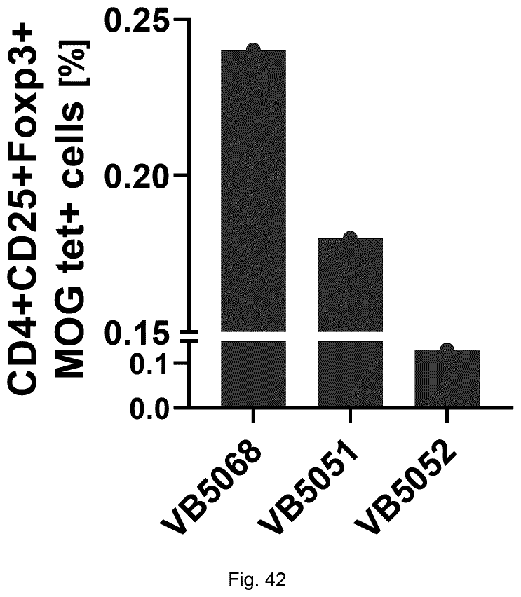

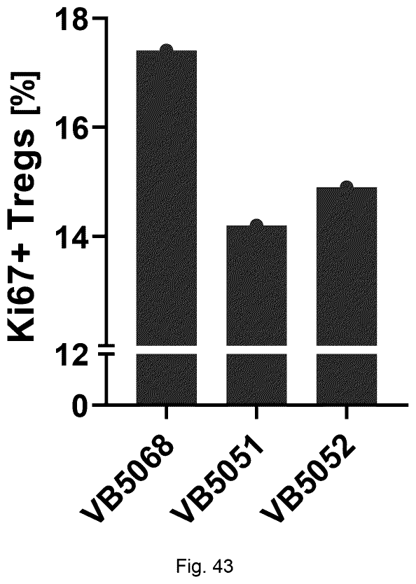

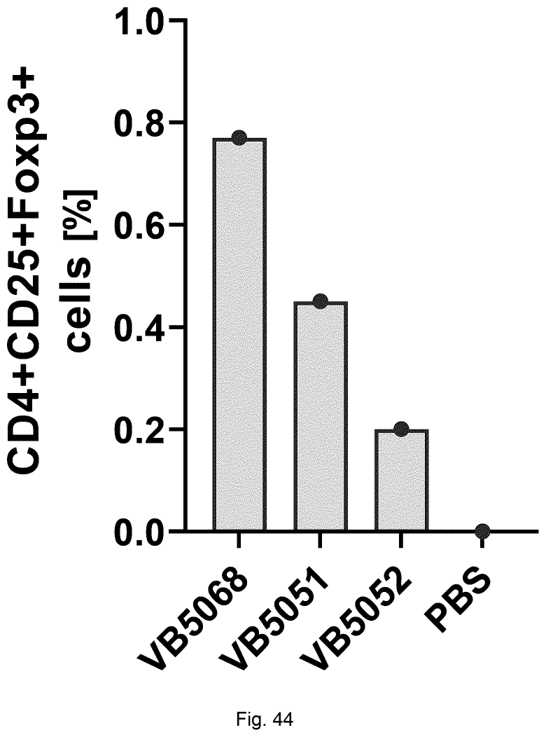

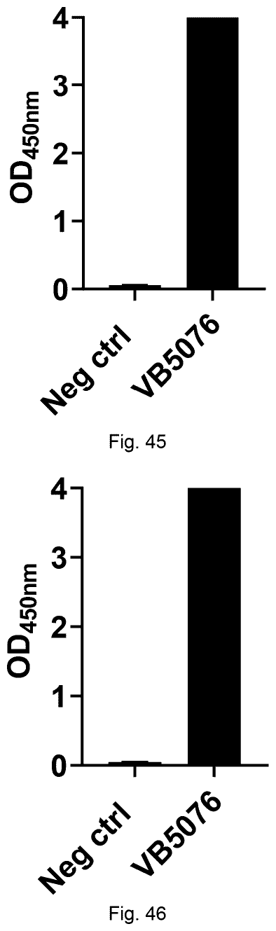

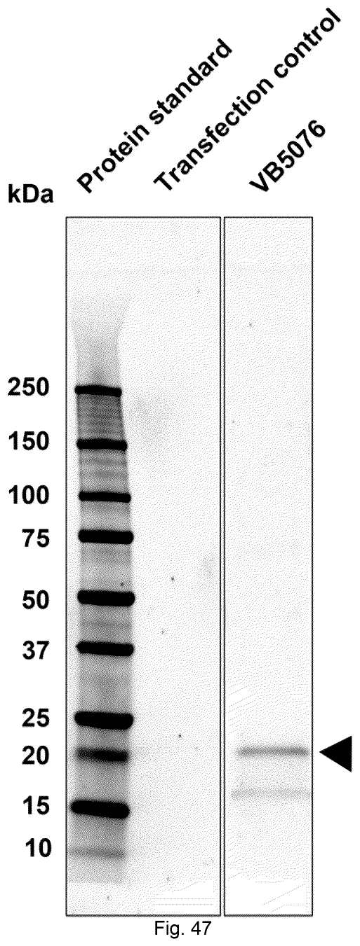

As shown in FIG. 10, a higher percentage of MOG (35-55)-specific Foxp3+ cells was detected following a single administration of VB5049* compared to VB5052* and VB5051*. For VB5052*, the observed MOG-specific Tregs are the result of a natural feedback loop to an inflammatory immune response elicited by VB5052* (as discussed in more detail in Example 5).

Example 3 thus shows that VB5049* induced a higher anti-inflammatory to inflammatory cytokine ratio (IL-10/IFN-γ) and shows a lack of inflammatory IFN-γ production, compared the pro-inflammatory control construct VB5052*. Example 3 further shows that VB5049* induces a higher number of MOG (35-55)-specific Foxp3+ cells compared to VB5052* or VB5051*. Altogether, these results indicate that administration of VB5049* elicited a greater antigen-specific tolerogenic response compared to administration of VB5051* and VB5052*.

### Example 4

DNA vectors were designed and produced, comprising nucleotide sequences encoding MOG (27-63) and further encoding the following elements/units:

TABLE 5

| | VB5062 | VB5063 | VB5064 | VB5065 | VB5048 |
|---|---|---|---|---|---|
| Elements/ | SEQ ID | SEQ ID | SEQ ID | SEQ ID | SEQ ID |
| units | NO: 20 | NO: 21 | NO: 22 | NO: 23 | NO: 35 |
| Signal | As in Table 4 | | | | |
| peptide | | | | | |
| Targeting | As in Table 4 | | | | |
| unit | | | | | |
| Dimeriza- | As in Table 4 | | | | |
| tion unit | | | | | |
| Unit linker | As in Table 4 | | | | |
| Antigenic | MOG (27-63) | | | | |
| unit | SEQ ID NO: 19 | | | | |
| Co- | T2A peptide | | | | NA |
| expression | SEQ ID NO: 6 | | | | |
| element | | | | | |
| Signal | Natural | Natural | Natural | Natural | NA |
| peptide for | leader | leader | leader | leader | |
| immuno- | sequence | sequence | sequence | sequence | |
| inhibitory | murine | murine | murine | murine | |
| compound | TGF-β1 | CTLA-4 | IL-2 | IFN-γ | |
| | SEQ ID | SEQ ID | SEQ ID | SEQ ID | |
| | NO: 24 | NO: 26 | NO: 28 | NO: 30 | |
| Immuno- | Murine | Murine | Murine | Murine | NA |
| inhibitory | TGF-β1 | CTLA-4 | IL-2 | IFN-γ | |
| compound | SEQ ID | extracellular | SEQ ID | SEQ ID | |
| | NO: 25 | domain | NO: 29 | NO: 31 | |
| | | SEQ ID | | | |
| | | NO: 27 | | | |

In addition, DNA vectors VB5049 (SEQ ID NO: 32), VB5052 (SEQ ID NO: 33) and VB501 (SEQ ID NO: 34) were designed and produced. These are identical to VB5049*, VB5052* and VB501* (Table 4) but comprise a nucleotide sequence encoding MOG (27-63) with SEQ ID NO: 18, instead of comprising a nucleotide sequence encoding MOG (27-63)* with SEQ ID NO: 16.

The DNA vectors VB5049, VB5052 and VB5062-VB5065 encode the same first polypeptide comprising an antigenic unit with MOG (27-63):
VB5049 encodes in addition mIL-10, which is expressed as a separate molecule
VB5062 encodes in addition mTGF-β1, which is expressed as a separate molecule
VB5063 encodes in addition the extracellular domain of mCTLA-4, which is expressed as a separate molecule
VB5064 encodes in addition mIL-2, which is expressed as a separate molecule
VB5065 encodes in addition mIFN-γ, which is expressed as a separate molecule
VB5048 encodes only the first polypeptide, but no immunoinhibitory compound. It serves as a comparison to the vectors of the invention
VB5052 is the “pro-inflammatory” version of the afore-listed DNA vectors and serves as comparison to the vectors of the invention
VB5051 only encodes MOG (27-63) and serves as a comparison to the vectors of the invention

### Characterization of Expression and Secretion of the Proteins Encoded by DNA Vectors

Expression and secretion of the proteins encoded by VB5049, VB5052, VB5062, VB5063, VB5064 and VB5065 was characterized as follows:

Briefly, Expi293F cells (1.7×10⁶ cells/ml, 1 ml) were seeded in a 96-well culture plate. The cells were transfected with 0.64 μg/ml plasmid DNA using ExpiFectamine 293 Reagent (Thermo Fisher Sci.), and the plates were incubated on an orbital shaker (3 mm diameter, 900 rpm) in a humidified CO₂ cell incubator (8% CO₂, 37° C.). Supernatants were harvested 72 hours post transfection.

The secreted first polypeptides/dimeric proteins were characterized in a sandwich ELISA of the supernatants using antibodies against MOG (capture antibody, mouse anti-MOG antibody, 0.25 μg/ml, 100 μl/well, sc-73330, Santa Cruz Biotechnology) and hIgG CH3 domain (detection antibody, mouse anti-human IgG Fc secondary antibody, biotin, 0.1 μg/ml, 100 μl/well, 05-4240, Invitrogen). As shown in FIG. 11, all first polypeptides/dimeric proteins were highly expressed and secreted.

The expression and secretion of the encoded immunoinhibitory compounds mIL-10, mTGF-β1, mCTLA-4, mIL-2 and mIFN-γ was characterized by sandwich ELISA using antibodies against mIL-10, hTGF-β1 (also binds to mTGF-β1), mCTLA-4, mIL-2 and mIFN-γ, respectively. The results are shown in FIGS. 12A-12E.

The following antibodies were used:
(A) Capture antibody: 1 μg/ml rat anti-murine IL-10 antibody, 100 μl/well, MAB417, R&D Systems. Detection antibody: 0.2 μg/ml, goat anti-murine IL-10 biotinylated antibody, 100 μl/well, BAF417, R&D Systems.
(B) Capture antibody: 2 μg/ml TGF-β 1 antibody, 100 μl/well, MAB2402, RD Systems. Detection antibody: 0.8 μg/ml chicken anti-human TGF-β1 biotinylated antibody, 100 μl/well, BAF240, RD Systems.
(C) Capture antibody: 0.8 μg/ml goat anti-murine CTLA-4 antibody, 100 μl/well, AF476, RD Systems. Detection antibody: 0.8 μg/ml goat anti-murine CTLA-4 biotinylated antibody, 100 μl/well, BAF476, RD Systems.
(D) Capture antibody: 2 μg/ml rat anti-murine IL-2 antibody, 100 μl/well, 503701, BioLegend. Detection antibody: 2 μg/ml rat anti-murine IL-2 biotinylated antibody, 100 μl/well, 503803, BioLegend.
(E) Capture antibody: 2 μg/ml rat anti-murine IFN-γ antibody, 100 μl/well, 505802, BioLegend. Detection antibody: 2 μg/ml rat anti-murine IFN-γ biotinylated antibody, 100 μl/well, 505704, BioLegend.

As shown in FIGS. 12A-E, all five immunoinhibitory compounds were expressed from their respective vectors and secreted.

The expression and secretion of the first polypeptides/dimeric proteins and the immunoinhibitory compounds as separate proteins was verified by sandwich ELISA with antibodies against MOG and the immunoinhibitory compound mIL-10, mTGF-β1, mCTLA-4 and mIL-2, respectively. The results are shown in FIGS. 13A-13D

The following antibodies were used: Capture antibody: mouse anti-MOG antibody, 0.25 μg/ml, 100 μl/well, sc-73330, Santa Cruz Biotechnology. Detection antibody:
(A) 0.2 μg/ml goat anti-mouse anti-IL-10 biotinylated antibody, 100 μl/well, BAF417, R&D Systems.
(B) 0.8 μg/ml chicken anti-human TGF-β1 biotinylated antibody, 100 μl/well, BAF240, RD Systems.
(C) 0.8 μg/ml goat anti-mouse CTLA-4 biotinylated antibody, 100 μl/well, BAF476, RD Systems.
(D) 2 μg/ml rat anti-mouse IFN-γ biotinylated antibody, 100 μl/well, 505704, BioLegend

As shown in FIGS. 13A-D, the first polypeptide and the immunoinhibitory compound are not expressed as a fusion protein but expressed and secreted as separate molecules.

Characterization of the Intact Proteins Expressed from DNA Vectors

Western blot analysis was performed on supernatant samples from transfected Expi293F cells to further characterize the proteins encoded by VB5049, VB5062, VB5063 and VB5064, encoding identical first polypeptides but different immunoinhibitory compounds. VB5048, encoding the same first polypeptide as the aforementioned DNA vectors but no immunoinhibitory compound was included as comparison.

The samples were prepared by mixing 14 μl supernatant from transfected Expi293F cells with 5 μl 4× Laemmli sample buffer (Bio-Rad) with 1 μl DTT (Cayman Chemical) or 1 μl ultrapure water for reducing and non-reducing conditions, respectively (scale-up of total sample volume with the given ratio). The samples (reduced or non-reduced) were heated at 70° C. for 10 minutes before added to 4%-20% Criterion TGX Stain-Free precast gels (Bio-Rad). SDS-PAGE was performed in 1× Tris/Glycine/SDS running buffer (Bio-Rad) with a Precision Plus Protein All Blue Prestained protein standard (Bio-Rad). Proteins were transferred from the gel onto EtOH activated low fluorescence (LF) 0.45 μm PVDF membranes (Bio-Rad) by using the Trans-Blot Turbo semi-dry transfer system (Bio-Rad). PVDF membranes were blocked in EveryBlot buffer (Bio-Rad) for 5 min and probed with mouse anti-MOG (sc-73330, Santa Cruz Biotechnology), rat anti-murine IL-10 (MAB417, R&D Systems), goat anti-murine CTLA-4 (AF467, R&D Systems) or rat anti-murine IL-2 (503702) to detect MOG, mIL-10, mCTLA-4 or mIL-2, respectively. The membranes were incubated with fluorochrome-conjugated species-specific secondary antibodies for 1 h at room temperature, and then washed and dried. For mIL10 detection in Dylight channel 488, membranes were re-probed with Dylight-488 secondary antibody. Membranes were reactivated in ethanol and TBST. Membranes were blocked, incubated with Dylight 488-conjugated secondary antibodies for 1 h at room temperature, and then washed and dried. Images were acquired by using a ChemiDoc™ MP Imaging System. Results are shown in FIG. 14.

The western blot analysis confirmed the ELISA results, demonstrating that VB5049, VB5062, VB5063 and VB5064 express two proteins: a first polypeptide/dimeric protein (FIGS. 14A and B, reduced and non-reduced conditions) and an immunoinhibitory compound as a second protein, i.e. mIL-10 (FIG. 14C), mCTLA-4 (FIG. 14D) or mIL-2 (FIG. 14E). Importantly, no additional bands were observed for the anti-IL-10, anti-CTLA-4 and anti-IL-2 probed membranes, demonstrating successful ribosome skipping at the T2A sequence, resulting in expression of the first polypeptide and an immunoinhibitory compound as separate proteins from a single DNA plasmid.

Taken together, the ELISA and western blot data demonstrate that intact dimeric proteins, comprising a targeting unit, dimerization unit and antigenic unit, can be co-expressed from a DNA plasmid together with an immunoinhibitory compound by using a T2A peptide as co-expression element.

Characterization of Expression and Secretion of the MOG (27-63) Peptide from the Vector VB5051

Protein expression and secretion of MOG (27-63) encoded by the vector VB5051 was determined as previously described in this Example 4.

The secretion of the MOG (27-63) peptide was characterized by direct ELISA, coating with the supernatant and detection using an antibody against MOG (capture antibody, 100 μl/well, 3.3 μg/ml mouse anti-MOG antibody, sc-73330, Santa Cruz Biotechnology). FIG. 15 shows that the MOG (27-63) peptide is expressed from VB5051 and secreted from mammalian cells transfected with said vector.

### Example 5

The tolerance-inducing ability of VB5049 was assessed in spleens from mice administered with VB5049 (see Example 4) and determined by the IL-10/IFN-γ ratio calculated from the IL-10 (an anti-inflammatory cytokine associated with immune tolerance) and IFN-γ (a marker for inducing an inflammatory immune response) signals produced in a dual color FluoroSpot assay following re-stimulation with MOG (35-55) peptide and by the detection of CD4+Foxp3+ T cells. Further, the absence of IFN-γ and IL-17 production, two pro-inflammatory cytokines known to be involved in MS pathology, was demonstrated. The results obtained were compared to the responses elicited by VB5052 or VB5051 administration (for units/elements and description of these vectors see Example 4).

The study design and method applied was similar to those described for VB5049* in Example 3, except that for VB5049, a different administration schedule was tested to validate the results obtained in Example 3, and to further extend the data beyond a single administration regimen.

Briefly, 50 μg of the DNA vectors VB5049, VB5051 or VB5052 was administered intramuscularly four times (DO, D3, D7 and D10) followed by electroporation, and spleens were harvested 14 days after the first administration. Spleens were mashed in a cell strainer to obtain single cell suspensions, and splenocytes were either re-stimulated for 44 hours with MOG (35-55) peptide or not re-stimulated, before tested for production of IFN-γ and IL-10 in a dual color FluoroSpot assay which was carried out as described in Example 3.

As shown in FIG. 16A, in non-re-stimulated splenocytes harvested from mice administered with VB5049, VB5051 or VB5052, some production of IL-10 was detected, while only low background levels of IFN-γ were observed. Upon MOG (35-55) re-stimulation of the splenocytes, no significant IFN-γ production was detected for the groups of mice which were administered with either VB5049 or VB5051, while in comparison, significantly elevated levels of IFN-γ were detected in splenocytes from mice administered with VB5052 (FIG. 16B). To avoid excess inflammation and for eventual resolution of inflammation, it is important that the production of pro-inflammatory cytokines, such as IFN-γ, is regulated by negative feedback mechanisms, including the production of anti-inflammatory cytokines such as IL-10 (see for instance Sugimoto et al., Front Immunol. 2016, Vol. 7, Article 160). Therefore, the increased level of IL-10 observed in response to VB5052 may be explained by such a feedback mechanism to control the inflammatory response induced. As shown in FIG. 17, a significantly higher IL-10/IFN-γ ratio was detected for VB5049 compared to VB5052, indicating a higher immunosuppressive potential of VB5049 compared to VB5052.

### Detection of Foxp3. IFN-γ and IL-17 by Flow Cytometry:

The presence of CD4+ T cells expressing Foxp3 was identified in the splenocyte population by flow cytometry. CD4+Foxp3+ T cells can suppress effector T cells and inflammatory immune responses, thereby maintaining self-tolerance. Flow cytometry was carried out as described in Example 3.

As shown in FIG. 18, a higher percentage of CD4+Foxp3+ T cells was detected in response to VB5049 administration compared to VB5051 administration, indicating higher levels of suppressive T cells induced by VB5049 compared to VB5051. The pro-inflammatory cytokines IFN-γ and IL-17 were not detected by flow cytometry in response to VB5049 administration as shown in FIGS. 19A and 19B, respectively. Contrary thereto, both pro-inflammatory cytokines were detected in response to administration of the pro-inflammatory comparison vector VB5052.

Example 5 thus shows that administration of VB5049, encoding a first polypeptide comprising an anti-DEC205 targeting unit, a dimerization unit and an antigenic comprising MOG (27-63) and further encoding IL-10, results in a higher anti-inflammatory to inflammatory cytokine ratio (IL-10/IFN-γ) compared to administration of VB5051 encoding only MOG (27-63). Moreover, administration of VB5049 results in a lack of inflammatory IFN-γ and IL-17 cytokine production and a higher percentage of CD4+Foxp3+ T cells, compared to VB5051. Altogether, these results indicate that VB5049 can elicit a greater antigen-specific tolerogenic response compared to the antigen alone (VB5051) in an anti-inflammatory manner as opposed to its pro-inflammatory version, VB5052. These results also validate the results of Example 3 and show that the tolerogenic properties of VB5049 are preserved following repetitive administration to mice.

### Example 6

The tolerance-inducing ability of VB5062 was assessed in spleens from mice administered with VB5062 (see Table 5/Example 4) and determined by the IL-10/IFN-γ ratio calculated from the IL-10 (an anti-inflammatory cytokine associated with immune tolerance) and IFN-γ (a marker for inducing an inflammatory immune response) signals produced in a dual color FluoroSpot assay following re-stimulation with MOG (35-55) peptide, and by the detection of Foxp3+ MOG(38-49)-specific regulatory T cells (Treg) detected ex vivo by use of MOG (38-49)-specific tetramers. The results obtained were compared to the immunogenicity elicited by VB5052 and tolerance-inducing ability of VB5051 (for units/elements and description of these vectors see Example 4).

Briefly, 50 μg of the DNA vectors VB5062, VB5051 or VB5052 was administered to mice (5 per group) intramuscularly followed by electroporation. Spleens were harvested 7 days after administration and mashed in a cell strainer to obtain single cell suspension. Splenocytes were either re-stimulated for 44 hours with MOG (35-55) peptide or not re-stimulated, before tested for production of IFN-γ and IL-10 in a dual color FluoroSpot assay which was carried out as described in Example 3.

As shown in FIG. 20A, in non-re-stimulated splenocytes harvested from mice administered with VB5062, VB5051 or VB5052, IL-10 production was detected, while only low background levels of IFN-γ were observed. Upon MOG (35-55) re-stimulation of the splenocytes, no significant IFN-γ production was detected for groups of mice which were administered with either VB5062 or VB5051 groups, while in comparison, significantly elevated levels of IFN-γ were detected in splenocytes from mice administered with VB5052 (FIG. 20B). The increased level of IL-10 observed in response to VB5052 may be explained by a potential feedback mechanism to control the inflammatory response induced, as described in Example 5. As shown in FIG. 21, a significantly higher IL-10/IFN-γ ratio was detected for VB5062 compared to VB5052, indicating a higher immunosuppressive potential of VB5062 compared to VB5052.

MOG (38-49) tetramer staining and flow cytometry was carried out as described in Example 3. Although similar IL-10 levels were detected for VB5062 and VB5051 in the dual color FluoroSpot assay (FIGS. 20A-B), a further single cell analysis showed increased percentage of CD4+MOG (38-49)-specific Foxp3+ cells in mice administered with VB5062 compared to mice administered with VB5051, as shown in FIG. 22.

Example 6 thus shows that administration of VB5062, encoding a first polypeptide comprising an anti-DEC205 targeting unit, a dimerization unit and an antigenic unit comprising MOG (27-63) and further encoding the immunoinhibitory compound TGFβ1, results in a higher anti-inflammatory to inflammatory cytokine ratio (IL-10/IFN-γ). Moreover, splenocytes from mice administered with VB5062 showed a lack of inflammatory IFN-γ cytokine production, compared to those obtained from mice administered with the pro-inflammatory version VB5052. Administration of VB5062 induced a higher percentage of MOG (38-49)-specific Foxp3+ cells compared to VB5051. Altogether, these results indicate that VB5062 can elicit a greater antigen-specific tolerogenic response compared to the antigen alone (VB5051) in an anti-inflammatory manner as opposed to its pro-inflammatory version, VB5052.

### Example 7

The tolerance-inducing ability of VB5063 was determined and compared to immunogenicity of VB5052 and tolerance-inducing ability of VB5051 (for units/elements and description of these vectors see Table 5/Example 4) as described in Example 6.

As shown in FIG. 23A, in non-re-stimulated splenocytes harvested from mice administered with VB5062, VB5051 or VB5052, production of IL-10 was detected, while only low background levels of IFN-γ were observed. Upon MOG (35-55) re-stimulation of the splenocytes, no significant IFN-γ production was detected for groups of mice which were administered with either VB5063 or VB5051, while in comparison, significantly elevated levels of IFN-γ were detected in splenocytes from mice administered with VB5052 (FIG. 23B). Significantly higher IL-10 levels were detected both in non-stimulated and MOG (35-55)-re-stimulated splenocytes harvested from mice administered with VB5063 compared to the levels detected in mice administered with VB5051. The increased level of IL-10 observed in response to VB5052 may be explained by a potential feedback mechanism to control the inflammatory response, as described in Example 5. As shown in FIG. 24, a significantly higher IL-10/IFN-γ ratio was detected for VB5063 compared to VB5052, indicating a higher immunosuppressive potential of VB5063 compared to VB5052.

As shown in FIG. 25, a higher percentage of MOG (38-49)-specific Foxp3+ cells was detected in response to VB5063 compared to VB5051, indicating increased generation of immunosuppressive Tregs in response to VB5063 administration.

Further, to determine the percentage of actively proliferating Treg cells induced in response to administration of VB5063, the expression of Ki67 (a nuclear marker strictly associated with dividing cells) was analyzed in splenocytes harvested from mice administered with VB5063 or VB5051. As shown in FIG. 26, a higher percentage of Ki67+ cells within the Treg (CD4+CD25+Foxp3+) population was detected ex vivo in mice administered with VB5063 compared to VB5051.

Example 7 thus shows that administration with VB5063, encoding a first polypeptide comprising an anti-DEC205 targeting unit, a dimerization unit and an antigenic unit comprising MOG (27-63) and further encoding the immunoinhibitory compound CTLA-4, results in a higher anti-inflammatory to inflammatory cytokine ratio (IL-10/IFN-γ). Moreover, splenocytes from mice administered with VB5063 showed a lack of inflammatory IFN-γ cytokine production compared to the pro-inflammatory version VB5052. Further, VB5063 induced a higher percentage of both MOG (38-49)-specific Foxp3+ and CD4+CD5+Foxp3+Ki67+ proliferating Treg cells compared to VB5051. Altogether, these results indicate that VB5063 can elicit a greater antigen-specific tolerogenic response compared to the antigen alone (VB5051) in an anti-inflammatory manner as opposed to its pro-inflammatory version, VB5052.

### Example 8

The tolerance-inducing ability of VB5064 was determined and compared to immunogenicity of VB5052 and tolerance-inducing ability of VB5051 (for units/elements and description of these vectors see Table 5/Example 4) as described in Example 6.

As shown in FIG. 27A, in non-re-stimulated splenocytes harvested from mice administered with VB5064, VB5051 or VB5052, production of IL-10 was detected, while only low background levels of IFN-γ were observed. Upon MOG (35-55) re-stimulation of the splenocytes, no significant IFN-γ production was detected for groups of mice which were administered with either VB5064 or VB5051, while in comparison, significantly elevated levels of IFN-γ were detected in splenocytes from mice administered with VB5052 (FIG. 27B). The increased level of IL-10 observed in response to VB5052 may be explained by a potential feedback mechanism to control the inflammatory response, as described in Example 5. As shown in FIG. 28, a significantly higher IL-10/IFN-γ ratio was detected for VB5064 compared to VB5052, indicating a higher immunosuppressive potential of VB5064 compared to VB5052.

Although similar IL-10 levels were detected for VB5064 and VB5051 in the dual color FluoroSpot assay (FIG. 27A-B), a higher percentage of MOG (38-49)-specific Foxp3+ cells was detected in response to VB5064 compared to VB5051 (FIG. 29).

Further, to determine the percentage of actively proliferating Treg cells induced in response to administration of VB5064, the expression of Ki67 (a nuclear marker strictly associated with dividing cells) was analyzed in splenocytes harvested from mice administered with VB5064 or VB5051. As shown in FIG. 30, a higher percentage of Ki67+ cells within the Treg (CD4+CD25+Foxp3+) population was detected ex vivo in the splenocytes of mice administered with VB5064 compared to VB5051.

Example 8 thus shows that administration with VB5064, encoding a first polypeptide comprising an anti-DEC205 targeting unit, a dimerization unit and an antigenic unit comprising MOG (27-63) and further encoding the immunoinhibitory compound IL-2, results in a higher anti-inflammatory to inflammatory cytokine ratio (IL-10/IFN-γ). Moreover, splenocytes from mice administered with VB5064 showed a lack of inflammatory IFN-γ cytokine production, compared to those obtained from mice administered with the pro-inflammatory version VB5052. Further, VB5064 induced a higher percentage of both MOG (38-49)-specific Foxp3+ and CD4+CD5+Foxp3+Ki67+ proliferating Treg cells compared to VB5051. Altogether, these results indicate that VB5064 can elicit a greater antigen-specific tolerogenic response compared to the antigen alone (VB5051) in an anti-inflammatory manner as opposed to its pro-inflammatory version, VB5052.

### Example 9

DNA vectors were designed and produced, comprising nucleotide sequences encoding MOG (27-63) and further encoding the following elements/units:

TABLE 6

| | VB5049 | VB5044 | VB5054 |
|---|---|---|---|
| | SEQ ID | SEQ ID | SEQ ID |
| Elements/units | NO: 32 | NO: 36 | NO: 37 |
| Signal peptide | As in Table 4 | | |
| Targeting unit | As in Table 4 | | |
| Dimerization unit | As in Table 4 | | |
| Unit linker | As in Table 4 | | |
| Antigenic unit | MOG (27-63) | | |
| | SEQ ID NO: 19 | | |
| Co-expression | T2A | | |
| element | SEQ ID NO: 6 | | |
| Signal peptide for | Natural leader sequence for mouse IL-10 | | |
| immuno-inhibitory | SEQ ID NO: 17 | | |
| compound 1 | | | |
| Immunoinhibitory | Mouse IL-10 | | |
| compound 1 | SEQ ID NO: 18 | | |
| Co-expression | NA | P2A | |
| element | | SEQ ID NO: 7 | |
| Signal peptide for | NA | Natural leader sequence for mouse | |
| immuno-inhibitory | | TGF-β1 SEQ ID NO: 24 | |
| compound 2 | | | |
| Immunoinhibitory | NA | Mouse TGF-β1 | |
| compound 2 | | SEQ ID NO: 25 | |
| Co-expression | NA | NA | T2A |
| element | | | SEQ ID NO: 6 |
| Signal peptide for | NA | NA | Natural leader |
| immuno-inhibitory | | | sequence for |
| compound 3 | | | mouse GM-CSF |
| | | | SEQ ID NO: 38 |
| Immunoinhibitory | NA | NA | Mouse GM-CSF |
| compound 3 | | | SEQ ID NO: 39 |

The DNA vectors VB5049, VB5044 and VB5054 encode the same first polypeptide comprising an antigenic unit with MOG (27-63) and further encode the following immunoinhibitory compounds which are expressed as separate molecules, due to the presence of the co-expression elements listed in Table 6:
VB5049: mIL-10 (serves as a control in the Western blot analysis)
VB 5044: mIL-10 and mTGF-β1
VB 5054: mIL-10 and mTGF-β1 and mGM-CSF

### Characterization of Expression and Secretion of the Proteins Encoded by DNA Vectors

Expression and secretion of the proteins encoded by VB5044 and VB5054 was characterized as described in Example 4. As shown in FIG. 31, the first polypeptide/dimeric protein from both vectors were highly expressed and secreted.

The expression and secretion of the encoded immunoinhibitory compounds mIL-10, mTGF-β1 and mGM-CSF was characterized by sandwich ELISA using antibodies against mIL-10, hTGF-β1 and mGM-CSF respectively. Results are shown in FIGS. 32A-C, which shows that all immunoinhibitory compounds encoded by VB5044 or VB5054 were expressed and secreted. Even a third immunoinhibitory compound encoded by the vector VB5054, mGM-CSF, was highly expressed and secreted (FIG. 32C).

The expression and secretion of the first polypeptide/dimeric protein and the immunoinhibitory compounds as separate proteins was verified by sandwich ELISA using antibodies against MOG and the immunoinhibitory compounds mIL-10, mTGF-β1 and mGM-CSF. The results show that the ribosomal skipping peptides T2A and P2A are highly effective, and that all first polypeptides/dimeric proteins and immunoinhibitory compounds were expressed and secreted as separate proteins (FIG. 33A-C).

Characterization of the Intact Proteins Expressed from VB5044 and VB5054 by Western blot

Western blot (WB) analysis was performed on supernatant from transfected Expi293F cells to further characterize the proteins encoded by VB5044 and VB5054. VB5049, encoding the same first polypeptide and the immunoinhibitory compound IL-10, was included as a control.

WB was performed as described in Example 4. PVDF membranes were probed with mouse anti-MOG (sc-73330, Santa Cruz Biotechnology), rat anti-murine IL-10 (MAB417, R&D Systems), rabbit anti-TGFβ1 (USB1042777-Biotin, United States Biological) or goat anti-murine GM-CSF (BAF415, R&D Systems), to detect the first polypeptide/dimeric protein, mIL-10, mTGF-β1 and mGM-CSF, respectively. Results are shown in FIGS. 34A-E.

The WB analysis confirmed the ELISA results, demonstrating that VB5044 and VB5054 express three and four proteins, respectively: a first polypeptide/dimeric protein (FIGS. 34A and B, reduced and non-reduced conditions) and two or three immunoinhibitory compounds, i.e. mIL-10 (FIG. 34C), mTGF-β1 (FIG. 34D) and mGM-CSF (FIG. 34E). Importantly, no additional bands were observed for the anti-IL-10, anti-TGF-β1 and anti-GM-CSF probed membranes, demonstrating successful ribosome skipping at the 2A sequences, resulting in expression of the first polypeptide and immunoinhibitory compounds as separate proteins from a single DNA plasmid.

Taken together, the ELISA and WB data demonstrate that intact dimeric proteins, comprising a targeting unit, dimerization unit and antigenic unit, can be co-expressed from a DNA vector together with several immunoinhibitory compounds by using different 2A peptides as co-expression elements.

### Example 10

DNA vectors were designed and produced, comprising nucleotide sequences encoding MOG (27-63) and further encoding the following elements/units:

TABLE 7

| | VB5068 | VB5069 | VB5070 |
|---|---|---|---|
| Elements/ | SEQ ID | SEQ ID | SEQ ID |
| units | NO: 40 | NO: 41 | NO:42 |
| Signal | Natural leader | Natural leader | Natural leader |
| peptide | sequence for | sequence for | sequence for |
| | mouse | mouse | mouse |
| | SCGB3A2 | VSIG-3 | PD-1 |
| | SEQ ID NO: 43 | SEQ ID NO: 45 | SEQ ID NO: 47 |
| Targeting | Mouse | Mouse VSIG-3 | Mouse PD-1 |
| unit | SCGB3A2 | extracellular | extracellular |
| | SEQ ID NO: 44 | domain | domain |
| | | SEQ ID NO: 46 | SEQ ID NO: 48 |

| | |
|---|---|
| Dimerization | As in Table 4 |
| unit | |
| Unit linker | As in Table 4 |
| Antigenic unit | MOG (27-63) |
| | SEQ ID NO: 19 |
| Co-expression | T2A peptide |
| element | SEQ ID NO: 6 |
| Signal peptide | Natural leader sequence murine IL-10 |
| for immuno- | SEQ ID NO: 17 |
| inhibitory | |
| compound | |
| Immuno- | Murine IL-10 |
| inhibitory | SEQ ID NO: 18 |
| compound | |

The DNA vectors VB5068, VB5069 and VB5070 encode a first polypeptide comprising an antigenic unit with MOG (27-63) and further encode the immunoinhibitory compound IL-10 which are expressed as separate molecules, due to the presence of the T2A peptide co-expression element. The first polypeptides encoded by these vectors comprise different targeting units:
VB5068: mSCGB3A2
VB5069: mVSIG3 extracellular domain
VB5070: mPD-1 extracellular domain

### Characterization of Expression and Secretion of the Proteins Encoded by DNA Vectors

Expression and secretion of the proteins encoded by VB5068, VB5069 and VB5070 was characterized as described in Example 4. As shown in FIG. 35, the first polypeptide/dimeric protein from all three vectors were highly expressed and secreted.

The expression and secretion of full-length first polypeptides/dimeric proteins encoded by VB5068 and VB5070 was verified by sandwich ELISA with antibodies against MOG and the respective targeting unit, mSCGB3A2 and mPD-1. FIGS. 36A and B show that the full-length proteins were highly expressed and secreted from both vectors.

Combined, these results demonstrate that the cells transfected with the DNA vectors VB5068 and VB5070 express and secrete proteins comprising a targeting unit (mSCGB3A2 or mPD-1), a CH3 containing dimerization unit, and the mouse MOG (27-63) antigen.

The secretion of the immunoinhibitory compound mIL-10 encoded by VB5068 and VB5069 was characterized by sandwich ELISA using antibodies against mouse IL-10. FIG. 37 shows that mIL-10 was highly expressed and secreted from both vectors.

The expression and secretion of the first polypeptide/dimeric protein and the immunoinhibitory compound IL-10 as separate proteins was verified by sandwich ELISA with antibodies against MOG and murine IL-10. The results show that the ribosomal skipping peptide T2A is highly effective, and that the first polypeptide/dimeric protein and the mIL-10 were expressed and secreted as separate proteins (FIG. 38).

Characterization of the Intact Proteins Expressed from VB5069 and VB5070

Western blot analysis was performed on supernatant from transfected Expi293F cells to further characterize the proteins encoded by the DNA vectors VB5069 and VB5070.

Western blot was performed as described in Example 4. PVDF membranes were probed with mouse anti-MOG (sc-73330, Santa Cruz Biotechnology) to detect the first polypeptide/dimeric protein and with rat anti-murine IL-10 (MAB417, R&D Systems) to detect the immunoinhibitory compound mIL-10. Results are shown in FIGS. 39A and B.

The western blot analysis confirmed the ELISA results, demonstrating that VB5069 and VB5070 express two proteins: a first polypeptide (FIG. 39A, reduced supernatant samples) and mIL-10 (FIG. 39B). Importantly, no additional bands were observed for the anti-IL-10-probed membrane, demonstrating successful ribosome skipping at the T2A sequence, resulting in expression of two separate proteins from a single DNA vector.

Taken together and in view of the previous Examples disclosed herein, the ELISA and WB data demonstrate that intact first polypeptides, comprising different targeting units than an scFv with specificity for murine CD205, can be co-expressed from a DNA vectors together with an immunoinhibitory compound (mIL-10) by using a T2A peptide as co-expression element.

### Example 11

The tolerance-inducing ability of VB5068 (see Table 7) was determined and compared to immunogenicity of VB5052 (pro-inflammatory version of VB5068) and tolerance-inducing ability of VB5051 (for units/elements and description of these vectors see Example 4) as described in Example 6.

As shown in FIG. 40A, in non-re-stimulated splenocytes harvested from mice administered with VB5068, VB5051 or VB5052, production of IL-10 was detected, while only low background levels of IFN-γ were observed. Upon MOG (35-55) re-stimulation of the splenocytes, no significant IFN-γ production was detected for groups of mice which were administered with VB5068 or VB5051, while in comparison, elevated levels of IFN-γ were detected in splenocytes from mice administered with VB5052 (FIG. 40B). The increased level of IL-10 observed in response to VB5052 may be explained by a potential feedback mechanism to control the inflammatory response, as described in Example 5.

Although similar IL-10 levels were detected in splenocytes from mice administered with either VB5068 or VB5051 in the dual color FluoroSpot assay (FIGS. 40A/B), a higher percentage of MOG (38-49)-specific Foxp3+ cells was detected in response to VB5068 compared to VB5051 by flow cytometry (FIG. 41).

The observed higher percentage of MOG (38-49)-specific Foxp3+ cells, indicating Tregs, detected in response to VB5068 (FIG. 41) was validated and confirmed by including CD25, an additional phenotypic marker to identify Tregs. Splenocytes harvested from mice administered with VB5068 and analyzed by flow cytometry showed higher levels of CD4+CD25+Foxp3+MOG (38-49)-tet+ Tregs compared to the levels detected in spleens from mice which were administered with either VB5051 or VB5052 (FIG. 42).

Further, to determine the percentage of actively proliferating Treg cells induced in response to administration of VB5064, the expression of Ki67 (a nuclear marker strictly associated with dividing cells) was analyzed in splenocytes harvested from mice administered with VB5068 or VB5051. As shown in FIG. 43, a higher percentage of Ki67+ cells within the Treg (CD4+CD25+Foxp3+) population was detected ex vivo in mice administered with VB5068 compared to VB5051 and VB5052.

To evaluate and confirm the percentage of Tregs induced and detected ex vivo (FIGS. 41 and 42) after administration of VB5068, the induction of Tregs was also assessed following MOG (35-55)-re-stimulation of the splenocytes. Splenocytes from mice administered with VB5068, VB5051 or VB5052 were re-stimulated with MOG (35-55) peptide for 16 h and analyzed by flow cytometry in an additional experiment. As shown in FIG. 44, a higher percentage of CD4+CD25+Foxp3+ Tregs was detected in splenocytes harvested from mice administered with VB5068 compared to those detected in spleens from mice administered with either VB5051 or VB5052.

Example 11 shows that administrating mice with VB5068, encoding a first polypeptide comprising an SCGB3A2 targeting unit, a dimerization unit and an antigenic unit comprising MOG (27-63) and further encoding the immunoinhibitory compound IL-10, results in a higher anti-inflammatory to inflammatory cytokine ratio (IL-10/IFN-γ). Moreover, splenocytes from mice administered with VB5068 showed a lack of inflammatory IFN-γ cytokine production compared to splenocytes from mice administered with the pro-inflammatory version VB5052. Further, VB5068 induced a higher percentage of both MOG (38-49)-specific Foxp3+ Tregs and CD4+CD5+Foxp3+Ki67+ proliferating Treg cells compared to both VB5051 and VB5052. Altogether, these results indicate that VB5064 can elicit a greater antigen-specific tolerogenic response compared to the antigen alone (VB5051) in an anti-inflammatory manner as opposed to its pro-inflammatory version, VB5052. Further, the data presented in Example 11 show the versatility of the vectors of the invention, demonstrating that various targeting units (anti-CD205 and SCGB3A2 targeting units) allow the targeting of the antigen comprised in the antigenic unit to APCs in a tolerogenic manner.

### Example 12

DNA vectors were designed and produced, comprising nucleotide sequences encoding T cell epitopes of the shrimp allergen tropomyosin from the species Metapenaeus ensis and further encoding the following elements/units:

TABLE 8

| | VB5076 |
|---|---|
| Elements/units | SEQ ID NO: 49 |
| Signal peptide | Murine Ig VH signal peptide |
| | SEQ ID NO: 2 |
| Targeting unit | scFv with specificity for murine CD205 |
| | SEQ ID NO: 15 |
| Dimerization unit | As in Table 4 |
| Unit linker | As in Table 4 |
| Antigenic unit | Met e 1 (241-260), (210-230), (136-155), |
| | (76-95), (46-65), and (16-35) |
| | SEQ ID NO: 50 |
| Co-expression | T2A peptide |
| element | SEQ ID NO: 6 |
| Signal peptide for | Natural leader sequence murine IL-10 |
| immunoinhibitory | SEQ ID NO: 17 |
| compound | |
| Immunoinhibitory | Murine IL-10 |
| compound | SEQ ID NO: 18 |

Tropomyosin is the major allergen in shellfish. Six major T cell epitopes were identified for tropomyosin from the shrimp species Metapenaeus ensis (Met e 1 allergen) in a Balb/c mouse model of Met e 1 hypersensitivity. Oral immunotherapy with peptides of the six T cell epitopes effectively reduced allergic responses towards shrimp tropomyosin (Wai et al., Int J Mol Sci 20(18), 4656, 2015).

The DNA vector VB5076 encodes a first polypeptide comprising an antigenic unit comprising T cell epitopes from Met e 1. The six T cell epitopes ((241-260), (210-230), (136-155), (76-95), (46-65), and (16-35)) are separated from each other by the T cell epitope linker GGGGSGGGGS. VB5076 further encodes the immunoinhibitory compound mIL 10.

### Characterization of Expression and Secretion of the Proteins Encoded by DNA Vector Vb5076

Expression and secretion of the proteins encoded by VB5076 was characterized as described in Example 4. As shown in FIG. 45, the first polypeptide/dimeric protein from VB5076 was expressed and secreted at a high level.

The secretion of the immunoinhibitory compound mIL-10 encoded by VB5076 was characterized by sandwich ELISA using antibodies against mouse IL-10. FIG. 46 shows that mIL-10 was highly expressed and secreted from VB5076.

Characterization of the Intact Proteins Expressed from VB5076

Western blot analysis was performed on supernatant from transfected Expi293F cells to further characterize the proteins encoded by VB5076. No commercially available antibody against the first polypeptide/dimeric protein expressed by VB5076 that is compatible with Western blotting could be identified; therefore, only the immunoinhibitory compound mIL-10 could be detected.

Western blot was performed as described in Example 4. The PVDF membrane was probed with rat anti-murine IL-10 (MAB417, R&D Systems) to detect the immunoinhibitory compound mIL-10. The result is shown in FIG. 47.

One major band at the expected molecular weight of IL-10 was observed for the anti-IL-10-probed membrane, demonstrating successful ribosome skipping at the T2A sequence, resulting in expression of mIL10 as a separate molecule from VB5076.

Taken together, the ELISA and western blot data demonstrate that first polypeptides/dimeric proteins comprising an antigenic unit with several T cell epitopes can be co-expressed from a DNA plasmid together with another protein (immunoinhibitory compound) by using a T2A peptide as co-expression element.

Sequence overview

SEQ ID NO: 1
Amino acid sequence of an embodiment of a dimerization unit consisting of hinge
region 1 from human IgG3 (amino acids 1-12), hinge region 4 from human IgG3 (amino
acids 13-27), glycine-serine linker (amino acids 28-37), CH3 domain from human lgG3
(amino acids 38-144)
ELKTPLGDTTHTEPKSCDTPPPCPRCPGGGSSGGGSGGQPREPQVYTLPPSREEMT
KNQVSLTCLVKGFYPSDIAVEWESSGQPENNYNTTPPMLDSDGSFFLYSKLTVDKSR
WQQGNIFSCSVMHEALHNRFTQKSLSLSPGK
SEQ ID NO: 2
Signal peptide of mIg VH
MNFGLRLIFLVLTLKGVQC
SEQ ID NO: 3
Amino acid sequence of VB5049*
Amino acid 1-19: mouse immunoglobulin heavy chain signal peptide; amino acid 20-265:
mouse single chain variable fragment (scFv) anti-DEC205; amino acid 266-409:
dimerization unit (SEQ ID NO: 1): hinge region 1 from human IgG3, hinge region 4 from
human IgG3, glycine-serine linker, CH3 domain from human IgG3; amino acid 410-414:
unit linker; amino acid 415-451 murine MOG 27-63 (MOG 35-55 underlined),
amino acid 452-454: linker; amino acid 455-472 co-expression element T2A peptide;
amino acid 473-490: signal peptide murine IL-10, natural leader sequence; amino acid
491-650: murine IL-10
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGGGSSGGGSGGQPREPQVYTLPPSREEMTKNQVSLTCLVKGFYPSDIAV
EWESSGQPENNYNTTPPMLDSDGSFFLYSKLTVDKSRWQQGNIFSCSVMHEALHNR
FTQKSLSLSPGKGLGGLSPGKNATGMEVGWYRSPFSRVVHLYRNGKDQDAEAQPG
SGEGRGSLLTCGDVEENPGPMPGSALLCCLLLLTGMRISRGQYSREDNNCTHFPVG
QSHMLLELRTAFSQVKTFFQTKDQLDNILLTDSLMQDFKGYLGCQALSEMIQFYLVEV
MPQAEKHGPEIKEHLNSLGEKLKTLRMRLRRCHRFLPCENKSKAVEQVKSDFNKLQD
QGVYKAMNEFDIFINCIEAYMMIKMKS
SEQ ID NO: 4
Amino acid sequence of VB5052*
Amino acid 1-23: signal peptide human CCL3L1; amino acid 24-93: human CCL3L1;
amino acid 94-237: dimerization unit (SEQ ID NO: 1): hinge region 1 from human IgG3,
hinge region 4 from human IgG3, glycine-serine linker, CH3 domain from human IgG3;
amino acid 238-242: unit linker; amino acid 243-279: murine MOG 27-63 (MOG 35-55
underlined)
MQVSTAALAVLLCTMALCNQVLSAPLAADTPTACCFSYTSRQIPQNFIADYFETSSQC
SKPSVIFLTKRGRQVCADPSEEWAVQKYVSDLELSAELKTPLGDTTHTEPKSCDTPPP
CPRCPGGGSSGGGSGGQPREPQVYTLPPSREEMTKNQVSLTCLVKGFYPSDIAVEW
ESSGQPENNYNTTPPMLDSDGSFFLYSKLTVDKSRWQQGNIFSCSVMHEALHNRFT
QKSLSLSPGKGLGGLSPGKNATGMEVGWYRSPFSRVVHLYRNGKDQDAEAQP
SEQ ID NO: 5
Amino acid sequence of VB5051*
Amino acid 1-19: mouse immunoglobulin heavy chain signal peptide; amino acid 20-56:
murine MOG 27-63 (MOG 35-55 underlined)
MNFGLRLIFLVLTLKGVQCSPGKNATGMEVGWYRSPFSRVVHLYRNGKDQDAEAQP
SEQ ID NO: 6
T2A
EGRGSLLTCGDVEENPGP
SEQ ID NO: 7
P2A
ATNFSLLKQAGDVEENPGP
SEQ ID NO: 8
E2A
QCTNYALLKLAGDVESNPGP
SEQ ID NO: 9
F2A
VKQTLNFDLLKLAGDVESNPGP
SEQ ID NO: 10
Nucleotide sequence encoding amino acids 1-27 of SEQ ID NO: 1
GAGCTCAAAACCCCACTTGGTGACACAACTCACACAGAGCCCAAATCTTGTGACA
CACCTCCCCCGTGCCCAAGGTGCCCA
SEQ ID NO: 11:
Nucleotide sequence encoding amino acids 38-144 of SEQ ID NO: 1
GGACAGCCCCGAGAACCACAGGTGTACACCCTGCCCCCATCCCGGGAGGAGATG
ACCAAGAACCAGGTCAGCCTGACCTGCCTGGTCAAAGGCTTCTACCCCAGCGAC
ATCGCCGTGGAGTGGGAGAGCAGCGGGCAGCCGGAGAACAACTACAACACCAC
GCCTCCCATGCTGGACTCCGACGGCTCCTTCTTCCTCTACAGCAAGCTCACCGTG
GACAAGAGCAGGTGGCAGCAGGGGAACATCTTCTCATGCTCCGTGATGCATGAG
GCTCTGCACAACCGCTTCACGCAGAAGAGCCTCTCCCTGTCTCCGGGTAAA
SEQ ID NO: 12:
Nucleotide sequence encoding the amino acid sequence of SEQ ID NO: 1
GAGCTCAAAACCCCACTTGGTGACACAACTCACACAGAGCCCAAATCTTGTGACA
CACCTCCCCCGTGCCCAAGGTGCCCAGGCGGTGGAAGCAGCGGAGGTGGAAGT
GGAGGACAGCCCCGAGAACCACAGGTGTACACCCTGCCCCCATCCCGGGAGGA
GATGACCAAGAACCAGGTCAGCCTGACCTGCCTGGTCAAAGGCTTCTACCCCAG
CGACATCGCCGTGGAGTGGGAGAGCAGCGGGCAGCCGGAGAACAACTACAACA
CCACGCCTCCCATGCTGGACTCCGACGGCTCCTTCTTCCTCTACAGCAAGCTCAC
CGTGGACAAGAGCAGGTGGCAGCAGGGGAACATCTTCTCATGCTCCGTGATGCA
TGAGGCTCTGCACAACCGCTTCACGCAGAAGAGCCTCTCCCTGTCTCCGGGTAAA
SEQ ID NO: 13
Signal peptide hCCL3L1
MQVSTAALAVLLCTMALCNQVLS
SEQ ID NO: 14
hCCL3L1
APLAADTPTACCFSYTSRQIPQNFIADYFETSSQCSKPSVIFLTKRGRQVCADPSEEW
VQKYVSDLELSA
SEQ ID NO: 15
scFv with specificity for murine CD205
DIQMTQSPSFLSTSLGNSITITCHASQNIKGWLAWYQQKSGNAPQLLIYKASSLQSGV
PSRFSGSGSGTDYIFTISNLQPEDIATYYCQHYQSFPWTFGGGTKLELKGGGGSGGG
GSGGGGSEVKLLESGGGLVQPGGSLRLSCAASGFTFNDFYMNWIRQPPGQAPEWL
GVIRNKGNGYTTEVNTSVKGRFTISRDNTQNILYLQMNSLRAEDTAIYYCARGGPYYY
SGDDAPYWGQGVMVTVSS
SEQ ID NO: 16
MOG (27-63)*
SPGKNATGMEVGWYRSPFSRVVHLYRNGKDQDAEAQP
SEQ ID NO: 17
Signal peptide mIL-10
MPGSALLCCLLLLTGMRI
SEQ ID NO: 18
mIL-10
SRGQYSREDNNCTHFPVGQSHMLLELRTAFSQVKTFFQTKDQLDNILLTDSLMQDFK
GYLGCQALSEMIQFYLVEVMPQAEKHGPEIKEHLNSLGEKLKTLRMRLRRCHRFLPC
ENKSKAVEQVKSDFNKLQDQGVYKAMNEFDIFINCIEAYMMIKMKS
SEQ ID NO: 19
MOG (27-63)
SPGKNATGMEVGWYRSPFSRVVHLYRNGKDQDAEQAP
SEQ ID NO: 20
VB5062
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGGGSSGGGSGGQPREPQVYTLPPSREEMTKNQVSLTCLVKGFYPSDIAV
EWESSGQPENNYNTTPPMLDSDGSFFLYSKLTVDKSRWQQGNIFSCSVMHEALHNR
FTQKSLSLSPGKGLGGLSPGKNATGMEVGWYRSPFSRVVHLYRNGKDQDAEQAPG
SGEGRGSLLTCGDVEENPGPMPPSGLRLLPLLLPLPWLLVLTPGRPAAGLSTCKTIDM
ELVKRKRIEAIRGQILSKLRLASPPSQGEVPPGPLPEAVLALYNSTRDRVAGESADPEP
EPEADYYAKEVTRVLMVDRNNAIYEKTKDISHSIYMFFNTSDIREAVPEPPLLSRAELR
LQRLKSSVEQHVELYQKYSNNSWRYLGNRLLTPTDTPEWLSFDVTGVVRQWLNQGD
GIQGFRFSAHCSCDSKDNKLHVEINGISPKRRGDLGTIHDMNRPFLLLMATPLERAQH
LHSSRHRRALDTNYCFSSTEKNCCVRQLYIDFRKDLGWKWIHEPKGYHANFCLGPCP
YIWSLDTQYSKVLALYNQHNPGASASPCCVPQALEPLPIVYYVGRKPKVEQLSNMIVR
SCKCS
SEQ ID NO: 21
VB5063
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGGGSSGGGSGGQPREPQVYTLPPSREEMTKNQVSLTCLVKGFYPSDIAV
EWESSGQPENNYNTTPPMLDSDGSFFLYSKLTVDKSRWQQGNIFSCSVMHEALHNR
FTQKSLSLSPGKGLGGLSPGKNATGMEVGWYRSPFSRVVHLYRNGKDQDAEQAPG
SGEGRGSLLTCGDVEENPGPMACLGLRRYKAQLQLPSRTWPFVALLTLLFIPVFSEAI
QVTQPSVVLASSHGVASFPCEYSPSHNTDEVRVTVLRQTNDQMTEVCATTFTEKNTV
GFLDYPFCSGTFNESRVNLTIQGLRAVDTGLYLCKVELMYPPPYFVGMGNGTQIYVID
PEPCPDSD
SEQ ID NO: 22
VB5064
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGGGSSGGGSGGQPREPQVYTLPPSREEMTKNQVSLTCLVKGFYPSDIAV
EWESSGQPENNYNTTPPMLDSDGSFFLYSKLTVDKSRWQQGNIFSCSVMHEALHNR
FTQKSLSLSPGKGLGGLMYSMQLASCVTLTLVLLVNSAPTSSSTSSSTAEAQQQQQQ
QQQQQQHLEQLLMDLQELLSRMENYRNLKLPRMLTFKFYLPKQATELKDLQCLEDEL
GPLRHVLDLTQSKSFQLEDAENFISNIRVTVVKLKGSDNTFECQFDDESATVVDFLRR
WIAFCQSIISTSPQ
SEQ ID NO: 23
VB5065
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGGGSSGGGSGGQPREPQVYTLPPSREEMTKNQVSLTCLVKGFYPSDIAV
EWESSGQPENNYNTTPPMLDSDGSFFLYSKLTVDKSRWQQGNIFSCSVMHEALHNR
FTQKSLSLSPGKGLGGLMNATHCILALQLFLMAVSGCYCHGTVIESLESLNNYFNSSGI
DVEEKSLFLDIWRNWQKDGDMKILQSQIISFYLRLFEVLKDNQAISNNISVIESHLITTFF
SNSKAKKDAFMSIAKFEVNNPQVQRQAFNELIRVVHQLLPESSLRKRKRSRC
SEQ ID NO: 24
Signal peptide mTGF-ß1
MPPSGLRLLPLLLPLPWLLVLTPGRPAAG
SEQ ID NO: 25
mTGF-ß1
LSTCKTIDMELVKRKRIEAIRGQILSKLRLASPPSQGEVPPGPLPEAVLALYNSTRDRV
AGESADPEPEPEADYYAKEVTRVLMVDRNNAIYEKTKDISHSIYMFFNTSDIREAVPEP
PLLSRAELRLQRLKSSVEQHVELYQKYSNNSWRYLGNRLLTPTDTPEWLSFDVTGVV
RQWLNQGDGIQGFRFSAHCSCDSKDNKLHVEINGISPKRRGDLGTIHDMNRPFLLLM
ATPLERAQHLHSSRHRRALDTNYCFSSTEKNCCVRQLYIDFRKDLGWKWIHEPKGYH
ANFCLGPCPYIWSLDTQYSKVLALYNQHNPGASASPCCVPQALEPLPIVYYVGRKPKV
EQLSNMIVRSCKCS
SEQ ID NO: 26
Signal peptide mCLTA-4
MACLGLRRYKAQLQLPSRTWPFVALLTLLFIPVFS
SEQ ID NO: 27
mCLTA-4
EAIQVTQPSVVLASSHGVASFPCEYSPSHNTDEVRVTVLRQTNDQMTEVCATTFTEK
NTVGFLDYPFCSGTFNESRVNLTIQGLRAVDTGLYLCKVELMYPPPYFVGMGNGTQIY
VIDPEPCPDSD
SEQ ID NO: 28
Signal peptide mIL-2
MYSMQLASCVTLTLVLLVNS
SEQ ID NO: 29
mIL-2
APTSSSTSSSTAEAQQQQQQQQQQQQHLEQLLMDLQELLSRMENYRNLKLPRMLTF
KFYLPKQATELKDLQCLEDELGPLRHVLDLTQSKSFQLEDAENFISNIRVTVVKLKGSD
NTFECQFDDESATVVDFLRRWIAFCQSIISTSPQ
SEQ ID NO: 30
Signal peptide mIFN-γ
MNATHCILALQLFLMAVSGCYC
SEQ ID NO: 31
mIFN-γ
HGTVIESLESLNNYFNSSGIDVEEKSLFLDIWRNWQKDGDMKILQSQIISFYLRLFEVLK
DNQAISNNISVIESHLITTFFSNSKAKKDAFMSIAKFEVNNPQVQRQAFNELIRVVHQLL
PESSLRKRKRSRC
SEQ ID NO: 32
VB5049
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGGGSSGGGSGGQPREPQVYTLPPSREEMTKNQVSLTCLVKGFYPSDIAV
EWESSGQPENNYNTTPPMLDSDGSFFLYSKLTVDKSRWQQGNIFSCSVMHEALHNR
FTQKSLSLSPGKGLGGLSPGKNATGMEVGWYRSPFSRVVHLYRNGKDQDAEQAPG
SGEGRGSLLTCGDVEENPGPMPGSALLCCLLLLTGMRISRGQYSREDNNCTHFPVG
QSHMLLELRTAFSQVKTFFQTKDQLDNILLTDSLMQDFKGYLGCQALSEMIQFYLVEV
MPQAEKHGPEIKEHLNSLGEKLKTLRMRLRRCHRFLPCENKSKAVEQVKSDFNKLQD
QGVYKAMNEFDIFINCIEAYMMIKMKS
SEQ ID NO: 33
VB5052
MQVSTAALAVLLCTMALCNQVLSAPLAADTPTACCFSYTSRQIPQNFIADYFETSSQC
SKPSVIFLTKRGRQVCADPSEEWAVQKYVSDLELSAELKTPLGDTTHTEPKSCDTPPP
CPRCPGGGSSGGGSGGQPREPQVYTLPPSREEMTKNQVSLTCLVKGFYPSDIAVEW
ESSGQPENNYNTTPPMLDSDGSFFLYSKLTVDKSRWQQGNIFSCSVMHEALHNRFT
QKSLSLSPGKGLGGLSPGKNATGMEVGWYRSPFSRVVHLYRNGKDQDAEQAP
SEQ ID NO: 34
VB5051
MNFGLRLIFLVLTLKGVQCSPGKNATGMEVGWYRSPFSRVVHLYRNGKDQDAEQAP
SEQ ID NO: 35
VB5048
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGGGSSGGGSGGQPREPQVYTLPPSREEMTKNQVSLTCLVKGFYPSDIAV
EWESSGQPENNYNTTPPMLDSDGSFFLYSKLTVDKSRWQQGNIFSCSVMHEALHNR
FTQKSLSLSPGKGLGGLSPGKNATGMEVGWYRSPFSRVVHLYRNGKDQDAEQAP
SEQ ID NO: 36
VB5044
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGGGSSGGGSGGQPREPQVYTLPPSREEMTKNQVSLTCLVKGFYPSDIAV
EWESSGQPENNYNTTPPMLDSDGSFFLYSKLTVDKSRWQQGNIFSCSVMHEALHNR
FTQKSLSLSPGKGLGGLSPGKNATGMEVGWYRSPFSRVVHLYRNGKDQDAEQAPG
SGEGRGSLLTCGDVEENPGPMPGSALLCCLLLLTGMRISRGQYSREDNNCTHFPVG
QSHMLLELRTAFSQVKTFFQTKDQLDNILLTDSLMQDFKGYLGCQALSEMIQFYLVEV
MPQAEKHGPEIKEHLNSLGEKLKTLRMRLRRCHRFLPCENKSKAVEQVKSDFNKLQD
QGVYKAMNEFDIFINCIEAYMMIKMKSGSGATNFSLLKQAGDVEENPGPMPPSGLRLL
PLLLPLPWLLVLTPGRPAAGLSTCKTIDMELVKRKRIEAIRGQILSKLRLASPPSQGEVP
PGPLPEAVLALYNSTRDRVAGESADPEPEPEADYYAKEVTRVLMVDRNNAIYEKTKDI
SHSIYMFFNTSDIREAVPEPPLLSRAELRLQRLKSSVEQHVELYQKYSNNSWRYLGNR
LLTPTDTPEWLSFDVTGVVRQWLNQGDGIQGFRFSAHCSCDSKDNKLHVEINGISPK
RRGDLGTIHDMNRPFLLLMATPLERAQHLHSSRHRRALDTNYCFSSTEKNCCVRQLYI
DFRKDLGWKWIHEPKGYHANFCLGPCPYIWSLDTQYSKVLALYNQHNPGASASPCC
VPQALEPLPIVYYVGRKPKVEQLSNMIVRSCKCS
SEQ ID NO: 37
VB5054
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGGGSSGGGSGGQPREPQVYTLPPSREEMTKNQVSLTCLVKGFYPSDIAV
EWESSGQPENNYNTTPPMLDSDGSFFLYSKLTVDKSRWQQGNIFSCSVMHEALHNR
FTQKSLSLSPGKGLGGLSPGKNATGMEVGWYRSPFSRVVHLYRNGKDQDAEQAPG
SGEGRGSLLTCGDVEENPGPMPGSALLCCLLLLTGMRISRGQYSREDNNCTHFPVG
QSHMLLELRTAFSQVKTFFQTKDQLDNILLTDSLMQDFKGYLGCQALSEMIQFYLVEV
MPQAEKHGPEIKEHLNSLGEKLKTLRMRLRRCHRFLPCENKSKAVEQVKSDFNKLQD
QGVYKAMNEFDIFINCIEAYMMIKMKSGSGATNFSLLKQAGDVEENPGPMPPSGLRLL
PLLLPLPWLLVLTPGRPAAGLSTCKTIDMELVKRKRIEAIRGQILSKLRLASPPSQGEVP
PGPLPEAVLALYNSTRDRVAGESADPEPEPEADYYAKEVTRVLMVDRNNAIYEKTKDI
SHSIYMFFNTSDIREAVPEPPLLSRAELRLQRLKSSVEQHVELYQKYSNNSWRYLGNR
LLTPTDTPEWLSFDVTGVVRQWLNQGDGIQGFRFSAHCSCDSKDNKLHVEINGISPK
RRGDLGTIHDMNRPFLLLMATPLERAQHLHSSRHRRALDTNYCFSSTEKNCCVRQLYI
DFRKDLGWKWIHEPKGYHANFCLGPCPYIWSLDTQYSKVLALYNQHNPGASASPCC
VPQALEPLPIVYYVGRKPKVEQLSNMIVRSCKCSGSGEGRGSLLTCGDVEENPGPM
WLQNLLFLGIVVYSLSAPTRSPITVTRPWKHVEAIKEALNLLDDMPVTLNEEVEVVSNE
FSFKKLTCVQTRLKIFEQGLRGNFTKLKGALNMTASYYQTYCPPTPETDCETQVTTYA
DFIDSLKTFLTDIPFECKKPVQK
SEQ ID NO: 38
Signal peptide mGM-CSF
MWLQNLLFLGIVVYSLS
SEQ ID NO: 39
mGM-CSF
APTRSPITVTRPWKHVEAIKEALNLLDDMPVTLNEEVEVVSNEFSFKKLTCVQTRLKIF
EQGLRGNFTKLKGALNMTASYYQTYCPPTPETDCETQVTTYADFIDSLKTFLTDIPFEC
KKPVQK
SEQ ID NO: 40
VB5068
MKLVSIFLLVTIGICGYSATALLINRLPVVDKLPVPLDDIIPSFDPLKMLLKTLGISVEHLVT
GLKKCVDELGPEASEAVKKLLEALSHLVELKTPLGDTTHTEPKSCDTPPPCPRCPGG
GSSGGGSGGQPREPQVYTLPPSREEMTKNQVSLTCLVKGFYPSDIAVEWESSGQPE
NNYNTTPPMLDSDGSFFLYSKLTVDKSRWQQGNIFSCSVMHEALHNRFTQKSLSLSP
GKGLGGLSPGKNATGMEVGWYRSPFSRVVHLYRNGKDQDAEQAPGSGEGRGSLLT
CGDVEENPGPMPGSALLCCLLLLTGMRISRGQYSREDNNCTHFPVGQSHMLLELRTA
FSQVKTFFQTKDQLDNILLTDSLMQDFKGYLGCQALSEMIQFYLVEVMPQAEKHGPEI
KEHLNSLGEKLKTLRMRLRRCHRFLPCENKSKAVEQVKSDFNKLQDQGVYKAMNEF
DIFINCIEAYMMIKMKS
SEQ ID NO: 41
VB5069
MTRRRSAPASWLLVSLLGVATSLEVSESPGSVQVARGQTAVLPCAFSTSAALLNLNVI
WMVIPLSNANQPEQVILYQGGQMFDGALRFHGRVGFTGTMPATNVSIFINNTQLSDT
GTYQCLVNNLPDRGGRNIGVTGLTVLVPPSAPQCQIQGSQDLGSDVILLCSSEEGIPR
PTYLWEKLDNTLKLPPTATQDQVQGTVTIRNISALSSGLYQCVASNAIGTSTCLLDLQV
ISPQPRSVELKTPLGDTTHTEPKSCDTPPPCPRCPGGGSSGGGSGGQPREPQVYTL
PPSREEMTKNQVSLTCLVKGFYPSDIAVEWESSGQPENNYNTTPPMLDSDGSFFLYS
KLTVDKSRWQQGNIFSCSVMHEALHNRFTQKSLSLSPGKGLGGLSPGKNATGMEVG
WYRSPFSRVVHLYRNGKDQDAEQAPGSGEGRGSLLTCGDVEENPGPMPGSALLCC
LLLLTGMRISRGQYSREDNNCTHFPVGQSHMLLELRTAFSQVKTFFQTKDQLDNILLT
DSLMQDFKGYLGCQALSEMIQFYLVEVMPQAEKHGPEIKEHLNSLGEKLKTLRMRLR
RCHRFLPCENKSKAVEQVKSDFNKLQDQGVYKAMNEFDIFINCIEAYMMIKMKS
SEQ ID NO: 42
VB5070
MWVRQVPWSFTWAVLQLSWQSGWLLEVPNGPWRSLTFYPAWLTVSEGANATFTCS
LSNWSEDLMLNWNRLSPSNQTEKQAAFCNGLSQPVQDARFQIIQLPNRHDFHMNILD
TRRNDSGIYLCGAISLHPKAKIEESPGAELVVTERILETSTRYPSPSPKPEGRFQGMEL
KTPLGDTTHTEPKSCDTPPPCPRCPGGGSSGGGSGGQPREPQVYTLPPSREEMTK
NQVSLTCLVKGFYPSDIAVEWESSGQPENNYNTTPPMLDSDGSFFLYSKLTVDKSRW
QQGNIFSCSVMHEALHNRFTQKSLSLSPGKGLGGLSPGKNATGMEVGWYRSPFSRV
VHLYRNGKDQDAEQAPGSGEGRGSLLTCGDVEENPGPMPGSALLCCLLLLTGMRIS
RGQYSREDNNCTHFPVGQSHMLLELRTAFSQVKTFFQTKDQLDNILLTDSLMQDFKG
YLGCQALSEMIQFYLVEVMPQAEKHGPEIKEHLNSLGEKLKTLRMRLRRCHRFLPCEN
KSKAVEQVKSDFNKLQDQGVYKAMNEFDIFINCIEAYMMIKMKS
SEQ ID NO: 43
Signal peptide mSCGB3A2
MKLVSIFLLVTIGICGYSATA
SEQ ID NO: 44
mSCGB3A2
LLINRLPVVDKLPVPLDDIIPSFDPLKMLLKTLGISVEHLVTGLKKCVDELGPEASEAVKK
LLEALSHLV
SEQ ID NO: 45
Signal peptide mVSIG3
MTRRRSAPASWLLVSLLGVATS
SEQ ID NO: 46
mVSIG3 extracellular domain
LEVSESPGSVQVARGQTAVLPCAFSTSAALLNLNVIWMVIPLSNANQPEQVILYQGGQ
MFDGALRFHGRVGFTGTMPATNVSIFINNTQLSDTGTYQCLVNNLPDRGGRNIGVTG
LTVLVPPSAPQCQIQGSQDLGSDVILLCSSEEGIPRPTYLWEKLDNTLKLPPTATQDQV
QGTVTIRNISALSSGLYQCVASNAIGTSTCLLDLQVISPQPRSV
SEQ ID NO: 47
Signal peptide mPD-1
MWVRQVPWSFTWAVLQLSWQSGWL
SEQ ID NO: 48
mPD-1 extracellular domain
LEVPNGPWRSLTFYPAWLTVSEGANATFTCSLSNWSEDLMLNWNRLSPSNQTEKQA
AFCNGLSQPVQDARFQIIQLPNRHDFHMNILDTRRNDSGIYLCGAISLHPKAKIEESPG
AELVVTERILETSTRYPSPSPKPEGRFQGM
SEQ ID NO: 49
VB5076
MNFGLRLIFLVLTLKGVQCDIQMTQSPSFLSTSLGNSITITCHASQNIKGWLAWYQQKS
GNAPQLLIYKASSLQSGVPSRFSGSGSGTDYIFTISNLQPEDIATYYCQHYQSFPWTF
GGGTKLELKGGGGSGGGGSGGGGSEVKLLESGGGLVQPGGSLRLSCAASGFTFND
FYMNWIRQPPGQAPEWLGVIRNKGNGYTTEVNTSVKGRFTISRDNTQNILYLQMNSL
RAEDTAIYYCARGGPYYYSGDDAPYWGQGVMVTVSSELKTPLGDTTHTEPKSCDTP
PPCPRCPGGGSSGGGSGGQPREPQVYTLPPSREEMTKNQVSLTCLVKGFYPSDIAV
EWESSGQPENNYNTTPPMLDSDGSFFLYSKLTVDKSRWQQGNIFSCSVMHEALHNR
FTQKSLSLSPGKGLGGLKEVDRLEDELVNEKEKYKSIGGGGSGGGGSAYKEQIKTLT
NKLKAAEARAEGGGGSGGGGSNQLKEARFLAEEADRKYDEVGGGGSGGGGSAALN
RRIQLLEEDLERSEERGGGGSGGGGSDLDQVQESLLKANNQLVEKDGGGGSGGGG
SEQQNKEANNRAEKSEEEVHNGSGEGRGSLLTCGDVEENPGPMPGSALLCCLLLLT
GMRISRGQYSREDNNCTHFPVGQSHMLLELRTAFSQVKTFFQTKDQLDNILLTDSLM
QDFKGYLGCQALSEMIQFYLVEVMPQAEKHGPEIKEHLNSLGEKLKTLRMRLRRCHR
FLPCENKSKAVEQVKSDFNKLQDQGVYKAMNEFDIFINCIEAYMMIKMKS
SEQ ID NO: 50
Met e 1
KEVDRLEDELVNEKEKYKSIGGGGSGGGGSAYKEQIKTLTNKLKAAEARAEGGGGSG
GGGSNQLKEARFLAEEADRKYDEVGGGGSGGGGSAALNRRIQLLEEDLERSEERGG
GGSGGGGSDLDQVQESLLKANNQLVEKDGGGGSGGGGSEQQNKEANNRAEKSEE
EVHN
SEQ ID NO: 51
hCTLA4 extracellular domain, signal peptide underlined
MACLGFQRHKAQLNLATRTWPCTLLFFLLFIPVFCKAMHVAQPAVVLASSRGIASFVC
EYASPGKATEVRVTVLRQADSQVTEVCAATYMMGNELTFLDDSICTGTSSGNQVNLTI
QGLRAMDTGLYICKVELMYPPPYYLGIGNGTQIYVIDPEPCPDSD
SEQ ID NO: 52
hPD-1 extracellular domain, signal peptide underlined
MQIPQAPWPVVWAVLQLGWRPGWFLDSPDRPWNPPTFSPALLVVTEGDNATFTCSF
SNTSESFVLNWYRMSPSNQTDKLAAFPEDRSQPGQDCRFRVTQLPNGRDFHMSVV
RARRNDSGTYLCGAISLAPKAQIKESLRAELRVTERRAEVPTAHPSPSPRPAGQFQTL
V
SEQ ID NO: 53
hIL-10, signal peptide underlined
MHSSALLCCLVLLTGVRASPGQGTQSENSCTHFPGNLPNMLRDLRDAFSRVKTFFQ
MKDQLDNLLLKESLLEDFKGYLGCQALSEMIQFYLEEVMPQAENQDPDIKAHVNSLGE
NLKTLRLRLRRCHRFLPCENKSKAVEQVKNAFNKLQEKGIYKAMSEFDIFINYIEAYMT
MKIRN
SEQ ID NO: 54
hTGFß-1, signal peptide underlined
MPPSGLRLLLLLLPLLWLLVLTPGRPAAGLSTCKTIDMELVKRKRIEAIRGQILSKLRLA
SPPSQGEVPPGPLPEAVLALYNSTRDRVAGESAEPEPEPEADYYAKEVTRVLMVETH
NEIYDKFKQSTHSIYMFFNTSELREAVPEPVLLSRAELRLLRLKLKVEQHVELYQKYSN
NSWRYLSNRLLAPSDSPEWLSFDVTGVVRQWLSRGGEIEGFRLSAHCSCDSRDNTL
QVDINGFTTGRRGDLATIHGMNRPFLLLMATPLERAQHLQSSRHRRALDTNYCFSST
EKNCCVRQLYIDFRKDLGWKWIHEPKGYHANFCLGPCPYIWSLDTQYSKVLALYNQH
NPGASAAPCCVPQALEPLPIVYYVGRKPKVEQLSNMIVRSCKCS
SEQ ID NO: 55
hIL-2, signal peptide underlined
MYRMQLLSCIALSLALVTNSAPTSSSTKKTQLQLEHLLLDLQMILNGINNYKNPKLTRM
LTFKFYMPKKATELKHLQCLEEELKPLEEVLNLAQSKNFHLRPRDLISNINVIVLELKGS
ETTFMCEYADETATIVEFLNRWITFCQSIISTLT
SEQ ID NO: 56
hGM-CSF, signal peptide underlined
MWLQSLLLLGTVACSISAPARSPSPSTQPWEHVNAIQEARRLLNLSRDTAAEMNETV
EVISEMFDLQEPTCLQTRLELYKQGLRGSLTKLKGPLTMMASHYKQHCPPTPETSCA
TQIITFESFKENLKDFLLVIPFDCWEPVQE
SEQ ID NO: 57
hIFN-y, signal peptide underlined
MKYTSYILAFQLCIVLGSLGCYCQDPYVKEAENLKKYFNAGHSDVADNGTLFLGILKN
WKEESDRKIMQSQIVSFYFKLFKNFKDDQSIQKSVETIKEDMNVKFFNSNKKKRDDFE
KLTNYSVTDLNVQRKAIHELIQVMAELSPAAKTGKRKRSQMLFRGRRASQ
SEQ ID NO: 58
hTGFß-2
MHYCVLSAFLILHLVTVALSLSTCSTLDMDQFMRKRIEAIRGQILSKLKLTSPPEDYPEP
EEVPPEVISIYNSTRDLLQEKASRRAAACERERSDEEYYAKEVYKIDMPPFFPSETVCP
VVTTPSGSVGSLCSRQSQVLCGYLDAIPPTFYRPYFRIVRFDVSAMEKNASNLVKAEF
RVFRLQNPKARVPEQRIELYQILKSKDLTSPTQRYIDSKVVKTRAEGEWLSFDVTDAV
HEWLHHKDRNLGFKISLHCPCCTFVPSNNYIIPNKSEELEARFAGIDGTSTYTSGDQKT
IKSTRKKNSGKTPHLLLMLLPSYRLESQQTNRRKKRALDAAYCFRNVQDNCCLRPLYI
DFKRDLGWKWIHEPKGYNANFCAGACPYLWSSDTQHSRVLSLYNTINPEASASPCC
VSQDLEPLTILYYIGKTPKIEQLSNMIVKSCKCS
SEQ ID NO: 59
hTGFß-3
MKMHLQRALVVLALLNFATVSLSLSTCTTLDFGHIKKKRVEAIRGQILSKLRLTSPPEPT
VMTHVPYQVLALYNSTRELLEEMHGEREEGCTQENTESEYYAKEIHKFDMIQGLAEH
NELAVCPKGITSKVFRFNVSSVEKNRTNLFRAEFRVLRVPNPSSKRNEQRIELFQILRP
DEHIAKQRYIGGKNLPTRGTAEWLSFDVTDTVREWLLRRESNLGLEISIHCPCHTFQP
NGDILENIHEVMEIKFKGVDNEDDHGRGDLGRLKKQKDHHNPHLILMMIPPHRLDNPG
QGGQRKKRALDTNYCFRNLEENCCVRPLYIDFRQDLGWKWVHEPKGYYANFCSGP
CPYLRSADTTHSTVLGLYNTLNPEASASPCCVPQDLEPLTILYYVGRTPKVEQLSNMV
VKSCKCS
SEQ ID NO: 60
hTGFß-1
ATGCCGCCCTCCGGGCTGCGGCTGCTGCCGCTGCTGCTACCGCTGCTGTGGCTA
CTGGTGCTGACGCCTGGCCGGCCGGCCGCGGGACTATCCACCTGCAAGACTATC
GACATGGAGCTGGTGAAGCGGAAGCGCATCGAGGCCATCCGCGGCCAGATCCT
GTCCAAGCTGCGGCTCGCCAGCCCCCCGAGCCAGGGGGAGGTGCCGCCCGGCC
CGCTGCCCGAGGCCGTGCTCGCCCTGTACAACAGCACCCGCGACCGGGTGGCC
GGGGAGAGTGCAGAACCGGAGCCCGAGCCTGAGGCCGACTACTACGCCAAGGA
GGTCACCCGCGTGCTAATGGTGGAAACCCACAACGAAATCTATGACAAGTTCAAG
CAGAGTACACACAGCATATATATGTTCTTCAACACATCAGAGCTCCGAGAAGCGG
TACCTGAACCCGTGTTGCTCTCCCGGGCAGAGCTGCGTCTGCTGAGGCTCAAGTT
AAAAGTGGAGCAGCACGTGGAGCTGTACCAGAAATACAGCAACAATTCCTGGCGA
TACCTCAGCAACCGGCTGCTGGCACCCAGCGACTCGCCAGAGTGGTTATCTTTTG
ATGTCACCGGAGTTGTGCGGCAGTGGTTGAGCCGTGGAGGGGAAATTGAGGGCT
TTCGCCTTAGCGCCCACTGCTCCTGTGACAGCAGGGATAACACACTGCAAGTGGA
CATCAACGGGTTCACTACCGGCCGCCGAGGTGACCTGGCCACCATTCATGGCAT
GAACCGGCCTTTCCTGCTTCTCATGGCCACCCCGCTGGAGAGGGCCCAGCATCT
GCAAAGCTCCCGGCACCGCCGAGCCCTGGACACCAACTATTGCTTCAGCTCCAC
GGAGAAGAACTGCTGCGTGCGGCAGCTGTACATTGACTTCCGCAAGGACCTCGG
CTGGAAGTGGATCCACGAGCCCAAGGGCTACCATGCCAACTTCTGCCTCGGGCC
CTGCCCCTACATTTGGAGCCTGGACACGCAGTACAGCAAGGTCCTGGCCCTGTA
CAACCAGCATAACCCGGGCGCCTCGGCGGCGCCGTGCTGCGTGCCGCAGGCGC
TGGAGCCGCTGCCCATCGTGTACTACGTGGGCCGCAAGCCCAAGGTGGAGCAGC
TGTCCAACATGATCGTGCGCTCCTGCAAGTGCAGCTGA
SEQ ID NO: 61
hTGFß-2
ATGCACTACTGTGTGCTGAGCGCTTTTCTGATCCTGCATCTGGTCACGGTCGCGC
TCAGCCTGTCTACCTGCAGCACACTCGATATGGACCAGTTCATGCGCAAGAGGAT
CGAGGCGATCCGCGGGCAGATCCTGAGCAAGCTGAAGCTCACCAGTCCCCCAGA
AGACTATCCTGAGCCCGAGGAAGTCCCCCCGGAGGTGATTTCCATCTACAACAGC
ACCAGGGACTTGCTCCAGGAGAAGGCGAGCCGGAGGGCGGCCGCCTGCGAGCG
CGAGAGGAGCGACGAAGAGTACTACGCCAAGGAGGTTTACAAAATAGACATGCC
GCCCTTCTTCCCCTCCGAAACTGTCTGCCCAGTTGTTACAACACCCTCTGGCTCA
GTGGGCAGCTTGTGCTCCAGACAGTCCCAGGTGCTCTGTGGGTACCTTGATGCC
ATCCCGCCCACTTTCTACAGACCCTACTTCAGAATTGTTCGATTTGACGTCTCAGC
AATGGAGAAGAATGCTTCCAATTTGGTGAAAGCAGAGTTCAGAGTCTTTCGTTTGC
AGAACCCAAAAGCCAGAGTGCCTGAACAACGGATTGAGCTATATCAGATTCTCAA
GTCCAAAGATTTAACATCTCCAACCCAGCGCTACATCGACAGCAAAGTTGTGAAAA
CAAGAGCAGAAGGCGAATGGCTCTCCTTCGATGTAACTGATGCTGTTCATGAATG
GCTTCACCATAAAGACAGGAACCTGGGATTTAAAATAAGCTTACACTGTCCCTGCT
GCACTTTTGTACCATCTAATAATTACATCATCCCAAATAAAAGTGAAGAACTAGAAG
CAAGATTTGCAGGTATTGATGGCACCTCCACATATACCAGTGGTGATCAGAAAACT
ATAAAGTCCACTAGGAAAAAAAACAGTGGGAAGACCCCACATCTCCTGCTAATGTT
ATTGCCCTCCTACAGACTTGAGTCACAACAGACCAACCGGCGGAAGAAGCGTGCT
TTGGATGCGGCCTATTGCTTTAGAAATGTGCAGGATAATTGCTGCCTACGTCCACT
TTACATTGATTTCAAGAGGGATCTAGGGTGGAAATGGATACACGAACCCAAAGGG
TACAATGCCAACTTCTGTGCTGGAGCATGCCCGTATTTATGGAGTTCAGACACTCA
GCACAGCAGGGTCCTGAGCTTATATAATACCATAAATCCAGAAGCATCTGCTTCTC
CTTGCTGCGTGTCCCAAGATTTAGAACCTCTAACCATTCTCTACTACATTGGCAAA
ACACCCAAGATTGAACAGCTTTCTAATATGATTGTAAAGTCTTGCAAATGCAGCTA
A
SEQ ID NO: 62
hTGFß-3
ATGAAGATGCACTTGCAAAGGGCTCTGGTGGTCCTGGCCCTGCTGAACTTTGCCA
CGGTCAGCCTCTCTCTGTCCACTTGCACCACCTTGGACTTCGGCCACATCAAGAA
GAAGAGGGTGGAAGCCATTAGGGGACAGATCTTGAGCAAGCTCAGGCTCACCAG
CCCCCCTGAGCCAACGGTGATGACCCACGTCCCCTATCAGGTCCTGGCCCTTTAC
AACAGCACCCGGGAGCTGCTGGAGGAGATGCATGGGGAGAGGGAGGAAGGCTG
CACCCAGGAAAACACCGAGTCGGAATACTATGCCAAAGAAATCCATAAATTCGAC
ATGATCCAGGGGCTGGCGGAGCACAACGAACTGGCTGTCTGCCCTAAAGGAATT
ACCTCCAAGGTTTTCCGCTTCAATGTGTCCTCAGTGGAGAAAAATAGAACCAACCT
ATTCCGAGCAGAATTCCGGGTCTTGCGGGTGCCCAACCCCAGCTCTAAGCGGAA
TGAGCAGAGGATCGAGCTCTTCCAGATCCTTCGGCCAGATGAGCACATTGCCAAA
CAGCGCTATATCGGTGGCAAGAATCTGCCCACACGGGGCACTGCCGAGTGGCTG
TCCTTTGATGTCACTGACACTGTGCGTGAGTGGCTGTTGAGAAGAGAGTCCAACT
TAGGTCTAGAAATCAGCATTCACTGTCCATGTCACACCTTTCAGCCCAATGGAGAT
ATCCTGGAAAACATTCACGAGGTGATGGAAATCAAATTCAAAGGCGTGGACAATG
AGGATGACCATGGCCGTGGAGATCTGGGGCGCCTCAAGAAGCAGAAGGATCACC
ACAACCCTCATCTAATCCTCATGATGATTCCCCCACACCGGCTCGACAACCCGGG
CCAGGGGGGTCAGAGGAAGAAGCGGGCTTTGGACACCAATTACTGCTTCCGGTG
A
SEQ ID NO: 63
hIL-10
ATGCACAGCTCAGCACTGCTCTGTTGCCTGGTCCTCCTGACTGGGGTGAGGGCC
AGCCCAGGCCAGGGCACCCAGTCTGAGAACAGCTGCACCCACTTCCCAGGCAAC
CTGCCTAACATGCTTCGAGATCTCCGAGATGCCTTCAGCAGAGTGAAGACTTTCTT
TCAAATGAAGGATCAGCTGGACAACTTGTTGTTAAAGGAGTCCTTGCTGGAGGAC
TTTAAGGGTTACCTGGGTTGCCAAGCCTTGTCTGAGATGATCCAGTTTTACCTGGA
GGAGGTGATGCCCCAAGCTGAGAACCAAGACCCAGACATCAAGGCGCATGTGAA
CTCCCTGGGGGAGAACCTGAAGACCCTCAGGCTGAGGCTACGGCGCTGTCATCG
ATTTCTTCCCTGTGAAAACAAGAGCAAGGCCGTGGAGCAGGTGAAGAATGCCTTT
AATAAGCTCCAAGAGAAAGGCATCTACAAAGCCATGAGTGAGTTTGACATCTTCAT
CAACTACATAGAAGCCTACATGACAATGAAGATACGAAACTGA
SEQ ID NO: 64
hSCGB3A2, signal peptide underlined
MKLVTIFLLVTISLCSYSATAFLINKVPLPVDKLAPLPLDNILPFMDPLKLLLKTLGISVEH
LVEGLRKCVNELGPEASEAVKKLLEALSHLV
SEQ ID NO: 65
hSCGB3A2
ATGAAGCTGGTAACTATCTTCCTGCTGGTGACCATCAGCCTTTGTAGTTACTCTGC
TACTGCCTTCCTCATCAACAAAGTGCCCCTTCCTGTTGACAAGTTGGCACCTTTAC
CTCTGGACAACATTCTTCCCTTTATGGATCCATTAAAGCTTCTTCTGAAAACTCTGG
GCATTTCTGTTGAGCACCTTGTGGAGGGGCTAAGGAAGTGTGTAAATGAGCTGGG
ACCAGAGGCTTCTGAAGCTGTGAAGAAACTGCTGGAGGCGCTATCACACTTGGTG
TGA
SEQ ID NO: 66
hVSIG-3 extracellular domain, signal peptide underlined
MTSQRSPLAPLLLLSLHGVAASLEVSESPGSIQVARGQPAVLPCTFTTSAALINLNVIW
MVTPLSNANQPEQVILYQGGQMFDGAPRFHGRVGFTGTMPATNVSIFINNTQLSDTG
TYQCLVNNLPDIGGRNIGVTGLTVLVPPSAPHCQIQGSQDIGSDVILLCSSEEGIPRPT
YLWEKLDNTLKLPPTATQDQVQGTVTIRNISALSSGLYQCVASNAIGTSTCLLDLQVIS
PQPRNIG
SEQ ID NO: 67
hVSIG-3 extracellular domain
ATGACTTCTCAGCGTTCCCCTCTGGCGCCTTTGCTGCTCCTCTCTCTGCACGGTG
TTGCAGCATCCCTGGAAGTGTCAGAGAGCCCTGGGAGTATCCAGGTGGCCCGGG
GTCAGCCAGCAGTCCTGCCCTGCACTTTCACTACCAGCGCTGCCCTCATTAACCT
CAATGTCATTTGGATGGTCACTCCTCTCTCCAATGCCAACCAACCTGAACAGGTCA
TCCTGTATCAGGGTGGACAGATGTTTGATGGTGCCCCCCGGTTCCACGGTAGGG
TAGGATTTACAGGCACCATGCCAGCTACCAATGTCTCTATCTTCATTAATAACACT
CAGTTATCAGACACTGGCACCTACCAGTGCCTGGTCAACAACCTTCCAGACATAG
GGGGCAGGAACATTGGGGTCACCGGTCTCACAGTGTTAGTTCCCCCTTCTGCCC
CACACTGCCAAATCCAAGGATCCCAGGATATTGGCAGCGATGTCATCCTGCTCTG
TAGCTCAGAGGAAGGCATTCCTCGACCAACTTACCTTTGGGAGAAGTTAGACAAT
ACCCTCAAACTACCTCCAACAGCTACTCAGGACCAGGTCCAGGGAACAGTCACCA
TCCGGAACATCAGTGCCCTGTCTTCAGGTTTGTACCAGTGCGTGGCTTCTAATGC
TATTGGAACCAGCACCTGTCTTCTGGATCTCCAGGTTATTTCACCCCAGCCCAGG
AACATTGGA

### EMBODIMENTS

### 1. A Vector Comprising:

(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of a self-antigen, an allergen, an alloantigen or a xenoantigen; and
(b) one or more further nucleic acid sequences encoding one or more immunoinhibitory compounds, wherein the vector allows for the co-expression of the first polypeptide and the one or more immunoinhibitory compounds as separate molecules.
2. The vector according to embodiment 1, wherein the one or more immunoinhibitory compounds induce immune tolerance.
3. The vector according to embodiment 1, wherein the one or more immunoinhibitory compounds increase immune tolerance.
4. The vector according to embodiment 1, wherein the one or more immunoinhibitory compounds maintain immune tolerance.
5. The vector according to embodiment 1, wherein the one or more immunoinhibitory compounds induce immune tolerance and/or increase immune tolerance and/or maintain immune tolerance
6. The vector according to any of embodiments 1 to 5, wherein the one or more immunoinhibitory compounds favor the presentation of the one or more T cell epitopes in the antigenic unit in a tolerance inducing manner, such as promotes and/or supports the presentation of the one or more T cell epitopes in the antigenic unit in a tolerance inducing manner.
7. The vector according to any of embodiments 1 to 6, wherein the one or more immunoinhibitory compounds favor the induction of tolerance maintaining cells, such as promotes and/or supports the induction of tolerance maintaining cells.
8. The vector according to any of embodiments 1 to 7, wherein the one or more immunoinhibitory compounds help maintaining tolerance maintaining cells.
9. The vector according to any of embodiments 1 to 8, wherein the immunoinhibitory compound is an extracellular part, such as the extracellular domain, of an inhibitory checkpoint molecule.
10. The vector according to embodiment 9, wherein the inhibitory checkpoint molecule is selected from the group consisting of CLTA-4, PD-1, BTLA, LAG3, NOX2, SIGLEC7, SIGLEC9 and TIM-3.
11. The vector according to any of embodiments 9 to 10, wherein the inhibitory checkpoint molecule is a human inhibitory checkpoint molecule, preferably a human inhibitory checkpoint molecule selected from the group consisting of hCLTA-4, such as hCTLA-4 with SEQ ID NO: 51, hPD-1, such as hPD-1 with SEQ ID NO: 52, hBTLA, hLAG3, hNOX2, hSIGLEC7, hSIGLEC9 and hTIM-3.
12. The vector according to any of embodiments 1 to 8, wherein the immunoinhibitory compound is a cytokine selected from the group consisting of IL-10, TGF-β1, TGF-β2, TGF-β3, IL-27, IL-2, GM-CSF, FLT3L, IFN-γ, IL-37 and IL-35.
13. The vector according to embodiment 12, wherein the cytokine is a human cytokine selected from the group consisting of hIL-10, such as hIL-10 with SEQ ID NO: 53, hTGF-β1, such as hTGF-β1 with SEQ ID NO: 54, hTGF-32, hTGF-β3, hIL-27, hIL-2, such as hIL-2 with SEQ ID NO: 55, hGM-CSF, such as hGM-CSF with SEQ ID NO: 56, hFLT3L, hIFN-γ, such as hIFN-γ with SEQ ID NO: 57, hIL-37 and hIL-35.
14. The vector according to any of embodiments 1 to 13 comprising multiple further nucleic acid sequences encoding multiple immunoinhibitory compounds, such as 2, 3, 4, 5, 6, 7 or 8 immunoinhibitory compounds, such as 2, 3, 4, 5, 6, 7 or 8 different immunoinhibitory compounds.
15. The vector according to embodiment 14, wherein said multiple immunoinhibitory compounds are different immunoinhibitory compounds which generate or promote a tolerance-inducing environment on different levels.
16. The vector according to any of embodiments 14 to 15, wherein said different immunoinhibitory compounds induce tolerance and increase tolerance and maintain tolerance or induce tolerance and increase tolerance or induce tolerance and maintain tolerance.
17. The vector according to any of the previous embodiments, wherein said vector comprises one or more co-expression elements.
18. The vector according to embodiment 17, wherein said one or more co-expression elements cause the transcription of the first polypeptide and the one or more immunoinhibitory compounds on a single transcript and the independent translation into a separate first polypeptide and separate one or more immunoinhibitory compounds.
19. The vector according to any of embodiments 17 to 18, wherein the one or more co-expression elements are IRES elements or nucleic acid sequences encoding 2A self-cleaving peptides.
20. The vector according to any of embodiments 17 to 18, wherein said vector comprises more than one co-expression element which are IRES elements or nucleic acid sequences encoding 2A self-cleaving peptides or IRES elements and nucleic acid sequences encoding 2A self-cleaving peptides.
21. The vector according to any of embodiments 17 to 20, wherein the 2A self-cleaving peptide is selected from the group consisting of T2A peptide, P2A peptide, E2A peptide and F2A peptide.
22. The vector according to any of embodiments 17 to 21, wherein the 2A self-cleaving peptide is selected from the group consisting of T2A peptide having an amino acid sequence which has 80% to 100% sequence identity to the amino acid sequence with SEQ ID NO: 6, P2A peptide having an amino acid sequence which has 80% to 100% sequence identity to the amino acid sequence with SEQ ID NO: 7, E2A peptide having an amino acid sequence which has 80% to 100% sequence identity to the amino acid sequence with SEQ ID NO: 8 and F2A peptide having an amino acid sequence which has 80% to 100% sequence identity to the amino acid sequence with SEQ ID NO: 9.
23. The vector according to any of embodiments 17 to 22, wherein the 2A self-cleaving peptide is selected from the group consisting of T2A peptide having an amino acid sequence with SEQ ID NO: 6, P2A peptide having an amino acid sequence with SEQ ID NO: 7, E2A peptide having an amino acid sequence with SEQ ID NO: 8 and F2A peptide having an amino acid sequence with SEQ ID NO: 9.
24. The vector according to embodiment 17, wherein said one or more co-expression elements cause the transcription of the first polypeptide and the one or more immunoinhibitory compounds as separate transcripts.
25. The vector according to embodiment 24, wherein said one or more co-expression elements are bidirectional promoters.
26. The vector according to embodiment 24, wherein said one or more co-expression elements are promoters and wherein the vector comprises a separate promoter for each of the nucleic acid sequences encoding the first polypeptide and the one or more immunoinhibitory compounds.
27. The vector according to embodiment 24, wherein said one or more co-expression elements are bidirectional promoters and promoters.
28. The vector according to any of embodiments 17 to 27, wherein said vector comprises one or more co-expression elements selected from the group consisting of IRES elements, nucleic acid sequences encoding 2A self-cleaving peptides, bidirectional promoters and promoters.
29. The vector according to any of embodiments 1 to 28, wherein the antigenic unit comprises one or more T cell epitopes of a self-antigen.
30. The vector according to embodiment 29, wherein the antigenic unit comprises multiple T cell epitopes of a self-antigen.
31. The vector according to embodiment 29, wherein the antigenic unit comprises multiple T cell epitopes of multiple different self-antigens.
32. The vector according to any of embodiments 29 to 31, wherein the self-antigen is involved in multiple sclerosis.
33. The vector according to any of embodiments 29 to 32, wherein the one or more T cell epitopes are selected from the group consisting of MOG, such as MOG (35-55), MBP, scuh as MBP (84-104) and MBP (76-112), PLP, such as PLP (139-151), PLP (131-159) and PLP (178-191), MAG, MOBP, CNPase, S100β and transaldolase.
34. The vector according to any of embodiments 29 to 33, wherein the antigenic unit comprises one or more T cell epitopes selected from the group consisting of MOG (35-55), MOG (27-63), PLP (139-151), PLP (131-159), PLP (178-191), PLP (170-199), MBP (84-104) and MBP (76-112). 35. The vector according to any of embodiments 29 to 31, wherein the self-antigen is involved in type 1 diabetes mellitus. 36. The vector according to embodiment 35, wherein the self-antigen is selected from the group consisting of glutamic acid decarboxylase 65-kilodalton isoform (GAD65), insulin, IA-2, ZnT8, IGRP, ChgA, IAPP, peripherin, tetraspanin-7, GRP78, urocortin-3 and insulin gene enhancer protein isl-1.
37. The vector according to any of embodiments 29 to 31, wherein the self-antigen is involved in celiac disease.
38. The vector according to embodiment 37, wherein the self-antigen is selected from the group consisting of α-gliadin, γ-gliadin, such as α-gliadin (76-95), ω-gliadin, low molecular weight glutenin, high molecular weight glutenin, hordein, secalin and avenin b.
39. The vector according to any of embodiments 29 to 31, wherein the self-antigen is involved in rheumatoid arthritis.
40. The vector according to embodiment 39, wherein the self-antigen is selected from the group consisting of collagen, heat shock protein 60 (HSP60), Band 3, small nuclear ribonucleoprotein D1 (SmD1), acetylcholine receptor (AChR) and myelin protein zero (P0).
41. The vector according to any of embodiments 29 to 31, wherein the self-antigen is involved in a disease selected from the group consisting of multiple sclerosis, type 1 diabetes mellitus, celiac disease, rheumatoid arthritis, chronic inflammatory demyelinating polyradiculoneuropathy, Hashimoto's thyroiditis, Pemphigus foliaceus, Pemphigus vulgaris, thyroid eye disease, Grave's disease, primary biliary cirrhosis, myasthenia gravis, insulin-resistant diabetes, hemolytic anemia and psoriasis and/or wherein the antigenic unit comprises one or more T cell epitopes of one or more self-antigens selected from the group consisting of neurofascin 155, thyroid peroxidase, thyroglobulin, desmosome-associated glycoprotein, desmoglein 3 calcium binding protein (calsequestrin), thyroid stimulating hormone receptor, antimitochondrial antibodies (AMAs), antinuclear antibodies (ANA), Rim-like/membrane (RL/M), multiple nuclear dot (MND), acetylcholine receptor, insulin receptor, erythrocytes cathelicidin (LL-37), disintegrin-like and metalloprotease domain containing thrombospondin type 1 motif-like 5 (ADAMTSL5), phospholipase A2 group IVD (PLA2G4D), heterogeneous nuclear ribonucleoprotein A1 (hnRNP-A1), keratin 17, citrullinated proteins, homocitrullinated proteins and the Fc portion of IgG.
42. The vector according to any of embodiments 1 to 28, wherein the antigenic unit comprises one or more T cell epitopes of an allergen.
43. The vector according to embodiment 42, wherein the antigenic unit comprises multiple T cell epitopes of an allergen.
44. The vector according to embodiment 42, wherein the antigenic unit comprises multiple T cell epitopes of multiple different allergens.
45. The vector according to any of embodiments 42 to 44, wherein the allergen is a food allergen.
46. The vector according to any of embodiments 42 to 45, wherein the antigenic unit comprises one or more T cell epitopes of one or more allergens selected from the group consisting of shellfish allergen, cow's milk allergen, egg allergen, fish allergen, fruit allergen, wheat allergen, peanut allergen, tree nut allergen, soy allergen, seed allergen, buckwheat allergen, celery allergen, garlic allergen, gluten allergen, oat allergen, legumes allergen, maize allergen, milk allergen, mustard allergen, nuts allergen, poultry allergen, meat allergen, rice allergen and sesame allergen.
47. The vector according to any of embodiments 42 to 45, wherein the antigenic unit comprises one or more T cell epitopes of one or more shellfish allergens selected from the group consisting of tropomyosin, arginine kinase, myosin light chain, sarcoplasmic calcium binding protein, troponin C, Triose-phosphate isomerase and actin.
48. The vector according to embodiment 47, wherein the one or more T cell epitopes are selected from the group consisting of Pan b 1 T cell epitope (251-270), Met e 1 T cell epitope (241-260), Met e 1 T cell epitope (210-230), Met e 1 T cell epitope (136-155), Met e 1 T cell epitope (76-95), Met e 1 T cell epitope (46-65) and Met e 1 T cell epitope (16-35).
49. The vector according to any of embodiments 42 to 44, wherein the antigenic unit comprises one or more T cell epitopes of one or more allergens selected from the group consisting of bee venom allergen, vespid allergen, latex allergen, dust mite allergen, house dust mite allergen, storage dust mite allergen, cockroach allergen, mold allergen, fungal allergen, furry animal allergen, such as dog allergen, cat allergen and horse allergen, pollen allergen, such as grass pollen allergen, tree pollen allergen, weed pollen allergen, insect allergen and drug allergen.
50. The vector according to any of embodiments 42 to 44 and 49, wherein the antigenic unit comprises one or more T cell epitopes of a drug allergen of a drug selected from the group consisting of Factor VIII, insulin and therapeutic monoclonal antibody.
51. The vector according to any of embodiments 1 to 28, wherein the antigenic unit comprises one or more T cell epitopes of an alloantigen.
52. The vector according to embodiment 51, wherein the antigenic unit comprises multiple T cell epitopes of an alloantigen.
53. The vector according to embodiment 51, wherein the antigenic unit comprises multiple T cell epitopes of multiple different alloantigens.
54. The vector according to any of embodiments 1 to 28, wherein the antigenic unit comprises one or more T cell epitopes of a xenoantigen.
55. The vector according to embodiment 54, wherein the antigenic unit comprises multiple T cell epitopes of a xenoantigen.
56. The vector according to embodiment 54, wherein the antigenic unit comprises multiple T cell epitopes of multiple different xenoantigens.
57. The vector according to any of embodiments 1 to 56, wherein the wherein the antigenic unit comprises multiple T cell epitopes which are discrete T cell epitopes.
58. The vector according to any of embodiments 1 to 56, wherein the antigenic unit comprises multiple T cell epitopes which are minimal T cell epitopes comprised in one or more hotspots.
59. The vector according to any of embodiments 1 to 56, wherein the antigenic unit comprises multiple T cell epitopes from the group consisting of discrete T cell epitopes and minimal T cell epitopes comprised in one or more hotspots.
60. The vector according to any of the previous embodiments, wherein the one or more T cell epitopes have a length of from 7 to about 200 amino acids, such as from 7 to 150 amino acids, preferably of from 7 to 100 amino acids, e.g. from 9 to 100 amino acids or from 15 to 100 amino acids or from 9 to 60 amino acids or from 9 to 30 amino acids or from 15 to 60 of from 15 to 30 or from 20 to 75 amino acids or from 25 to 50 amino acids.
61. The vector according to embodiment 60, wherein the one or more T cell epitopes have a length suitable for presentation by MHC (major histocompatibility complex), such as a length of from 7 to 11 amino acids for MHC class I presentation or a length of about 15 amino acids for MHC class II presentation.
62. The vector according to any of the previous embodiments, wherein the antigenic unit comprises up to 3500 amino acids, such as from about 21 to about 2000 amino acids or from about 60 to 3500 amino acids, such as from about 80 or about 100 or about 150 amino acids to about 3000 amino acids, such as from about 200 to about 2500 amino acids, such as from about 300 to about 2000 amino acids or from about 400 to about 1500 amino acids or from about 500 to about 1000 amino acids.
63. The vector according to any of the previous embodiments, wherein the antigenic unit comprises 1 to 10 T cell epitopes such as 1, 2, 3, 4, 5, 6, 7, 8 or 9 or 10 T cell epitopes or 11 to 20 T cell epitopes, such as 11, 12, 13, 14, 15, 16, 17, 18, 19 or 20 T cell epitopes or 21 to 30 T cell epitopes, such as 21, 22, 23, 24, 25, 26, 27, 28, 29 or 30 T cell epitopes or 31 to 40 T cell epitopes, such as 31, 32, 33, 34, 35, 36, 37, 38, 39 or 40 T cell epitopes or 41 to 50 T cell epitopes, such as 41, 42, 43, 44, 45, 46, 47, 48, 49 or 50 T cell epitopes.
64. The vector according to any of the previous embodiments, wherein the antigenic unit comprises multiple discrete T cell epitopes which are separated by T cell epitope linkers.
65. The vector according to any of the previous embodiments, wherein the targeting unit is or comprises a moiety that interacts with surface molecules on the antigen-presenting cell without activating the cell.
66. The vector according to any of the previous embodiments, wherein the targeting unit is or comprises a moiety that interacts with surface molecules on the antigen-presenting cell without inducing maturation of the cell.
67. The vector according to any of embodiments 65 to 66, wherein the surface molecule is selected from the group consisting of TGFβ receptor, such as TGFβR1, TGFβR2, and TGFβR3, IL-10R, such as IL-10RA and IL-10RB, IL-2R, IL-4R, IL-6R, IL-11R, IL-13R, IL-27R, IL-35R, IL-37R, GM-CSFR, FLT3, CCR7, CD11b, CD11c, CD103, CD14, CD36, CD205, CD109, VISTA, MARCO, MHCII, CD83, SIGLEC, Clec10A (MGL), ASGR (ASGR1/ASGR2), CD80, CD86, Clec9A, Clec12A, Clec12B, DCIR2, Langerin, MR, DC-Sign, Treml4, Dectin-1, PDL1, PDL2, HVEM, CD163 and CD141.
68. The vector according to any of embodiments 65 to 66, wherein the surface molecule is a surface molecule present on human antigen-presenting cells and wherein said surface molecule is selected from the group consisting of hTGFβ receptor, such as hTGFβR1, hTGFβR2, and hTGFβR3, hIL-10R, such as hIL-10RA and hIL-10RB, hIL-2R, hIL-4R, hIL-6R, hIL-11R, hIL-13R, hIL-27R, hIL-35R, hIL-37R, hGM-CSFR, hFLT3, hCCR7, hCD11b, hCD11c, hCD103, hCD14, hCD36, hCD205, hCD109, hVISTA, hMARCO, hMHCII, hCD83, hSIGLEC, hClec10A (hMGL), hASGR (hASGR1/hASGR2), hCD80, hCD86, hClec9A, hClec12A, hClec12B, hDCIR2, hLangerin, hMR, hDC-Sign, hTreml4, hDectin-1, hPDL1, hPDL2, hHVEM, hCD163 and hCD141.
69. The vector according to any of embodiments 65 to 68, wherein the moiety is a natural ligand, an antibody or part thereof, such as a scFv, or a synthetic ligand.
70. The vector according to any of embodiments 65 to 68, wherein the moiety is a natural ligand selected from the group consisting of TGFβ, such as TGF-β1, TGF-β2 and TGF-β3, IL-10, IL-2, IL-4, IL-6, IL-11, IL-13, IL-27, IL-35, IL-37, GM-CSF, FLT3L, CCL19, CCL21, ICAM-1 (Intercellular Adhesion Molecule 1 also known as CD54), keratin, VSIG-3, preferably the extracellular domain of VSIG-3, SCGB3A2, CTLA-4, preferably the extracellular domain of CTLA-4, PD-1, preferably the extracellular domain of PD-1 and BTLA, preferably the extracellular domain of BTLA.
71. The vector according to any of embodiments 65 to 68, wherein the moiety is a human natural ligand selected from the group consisting of hTGFβ, hIL-10, hIL-2, such as hIL-2 with SEQ ID NO: 55, hIL-4, hIL-6, hIL-11, hIL-13, hIL-27, hIL-35, hIL-37, hGM-CSF, such as hGM-CSF with SEQ ID NO: 56, hFLT3L, hCCL19, hCCL21, hICAM-1 (Intercellular Adhesion Molecule 1 also known as CD54), hkeratin, hVSIG-3, preferably the extracellular domain of hVSIG-3, hSCGB3A2, hCTLA-4, preferably the extracellular domain of hCTLA-4, such as the extracellular domain of hCTLA4 with SEQ ID NO: 51, hPD-1, preferably the extracellular domain of PD-1, such as the extracellular domain of hPD-1 with SEQ ID NO: 52 and hBTLA, preferably the extracellular domain of hBTLA.
72. The vector according to any of embodiments 65 to 70, wherein the targeting unit is or comprises IL1-10, TGFβ, such as TGFβ-1, TGFβ-2 and TGFβ-3, SCGB3A2 or VSIG-3, preferably the extracellular domain of VSIG-3.
73. The vector according to any of embodiments 65 to 70, wherein the targeting unit is or comprises hIL1-10, hTGFβ, such as hTGFβ-1, hTGFβ-2 and hTGFβ-3, hSCGB3A2 or hVSIG-3, preferably the extracellular domain of hVSIG-3.
74. The vector according to any of the previous embodiments, wherein the multimerization unit is selected from the group consisting of dimerization unit, trimerization unit, such as a collagen-derived trimerization unit, such as a human collagen-derived trimerization domain, such as human collagen derived XVIII trimerization domain or human collagen XV trimerization domain or the C-terminal domain of T4 fibritin and tetramerization unit, such as a domain derived from p53 and wherein said multimerization unit optionally comprises a hinge region, such as hinge exon h1 and hinge exon h4, preferably hinge exon h1 and hinge exon h4 of IgG3.
75. The vector according to embodiment 74, wherein the vector comprises a hinge region which has the ability to form one or more covalent bonds.
76. The vector according to any of embodiments 74 to 75, wherein the hinge region is Ig derived.
77. The vector according to any of embodiments 74 to 76, wherein the multimerization unit is a dimerization unit and said dimerization unit further comprises another domain that facilitates dimerization.
78. The vector according to embodiment 77, wherein the other domain is an immunoglobulin domain, preferably an immunoglobulin constant domain.
79. The vector according to any of embodiments 77 to 78, wherein the other domain is a carboxyterminal C domain derived from IgG, preferably from IgG3.
80. The vector according to any of embodiments 77 to 79, wherein the dimerization unit further comprises a dimerization unit linker, such as glycine-serine rich linker, such as GGGSSGGGSG.
81. The vector according to embodiment 80, wherein the dimerization unit linker connects the hinge region and the other domain that facilitates dimerization.
82. The vector according to any of embodiments 77 to 81, wherein the dimerization unit comprises hinge exon h1 and hinge exon h4, a dimerization unit linker and a CH3 domain of human IgG3.
83. The vector according to embodiment 82, wherein the dimerization unit comprises an amino acid sequence having at least 80% sequence identity to the amino acid sequence SEQ ID NO: 1.
84. The vector according to embodiment 83, wherein the dimerization unit consists of an amino acid sequence having at least 80% sequence identity to the amino acid sequence of SEQ ID NO: 1.
85. The vector according to embodiment 84, wherein the dimerization unit consists of the amino acid sequence of SEQ ID NO: 1.
86. The vector according to any of the previous embodiments, wherein the first nucleic acid sequence encodes a first polypeptide which further comprises a unit liker that connects the antigenic unit to the multimerization unit, and wherein the unit linker is a non-immunogenic linker and/or flexible or rigid linker.
87. The vector according to any of the previous embodiments, wherein the first nucleic acid sequence encode a first polypeptide which further comprises a signal peptide.
88. The vector according to embodiment 87, wherein the signal peptide is the natural leader sequence of the protein which is the targeting unit.
89. The vector according to any of embodiments 87 to 88, wherein the signal peptide is selected from the group of consisting of human Ig VH signal peptide, signal peptide of hTGF-β1, signal peptide of hTGF-β2 signal peptide of hTGF-β3, signal peptide of hIL-10, signal peptide of hIL-2, signal peptide of hIL-4, signal peptide of hIL-6, signal peptide of hIL-11, signal peptide of hIL-13, signal peptide of hIL-27, signal peptide of hIL-35, signal peptide of hIL-37, signal peptide of hGM-CSF, signal peptide of hFLT3L, signal peptide of hCCL19, signal peptide of hCCL21, signal peptide of hICAM-1, signal peptide of hkeratin, signal peptide of hVSIG-3, signal peptide of hSCGB3A2, signal peptide of hCTLA-4, signal peptide of hPD-1 and signal peptide of hBTLA.
90. The vector according to any of the previous embodiments, wherein the one or more further nucleic acid sequences encode one or more immunoinhibitory compounds which further comprise a signal peptide.
91. The vector according to embodiment 90, wherein the signal peptide is the natural leader sequence of the immunoinhibitory compound.
92. The vector according to any of embodiments 90 to 91, wherein the signal peptide is selected from the group consisting of signal peptide selected from the group consisting of signal peptide of hCLTA-4, signal peptide of hPD-1, signal peptide of hBTLA, signal peptide of hLAG3, signal peptide of hNOX2, signal peptide of hSIGLEC7, signal peptide of hSIGLEC9, signal peptide of hTIM-3, signal peptide of hIL-10, signal peptide of hTGF-β1, signal peptide of hTGF-β2, signal peptide of hTGF-β3, signal peptide of hIL-27, signal peptide of hIL-2, signal peptide of hGM-CSF signal peptide of hFLT3L, signal peptide of hIFN-γ and signal peptide of hIL-37 and signal peptide of hIL-35.
93. The vector according to any of the previous embodiments, wherein the vector is a viral vector, such as an RNA viral vector or DNA viral vector or a plasmid, such as an RNA plasmid or DNA plasmid.
94. The vector according to any of the previous embodiments, wherein the vector is a DNA viral vector or a DNA plasmid, preferably a DNA plasmid.
95. A method of producing a vector as defined in any of embodiments 1 to 94, the method comprising:
a) transfecting cells in vitro with the vector;
b) culturing said cells;
c) optionally, lysing the cells to release the vector from the cells; and
d) collecting and optionally purifying the vector.
96. A host cell comprising a vector as defined in any of embodiments 1 to 94, such as a host cell selected from the group consisting of prokaryote cells, yeast cells, insect cells, higher eukaryotic cells such as cells from animals or humans.
97. A vector as defined in any of embodiments 1 to 94 for use as a medicament.
98. A pharmaceutical composition comprising the vector as defined in any of embodiments 1 to 94 and a pharmaceutically acceptable carrier or diluent.
99. The pharmaceutical composition according to embodiment 98, wherein the pharmaceutically acceptable carrier or diluent is selected from the group consisting of saline, buffered saline, such as PBS, dextrose, water, glycerol, ethanol, isotonic aqueous buffers and Tyrode's buffer and combinations thereof.
100. The pharmaceutical composition according to any of embodiments 98 to 99, wherein the composition further comprises a transfection agent.
101. The composition according to any of embodiments 98 to 100, wherein the composition further comprises a pharmaceutically acceptable amphiphilic block co-polymer comprising blocks of poly(ethylene oxide) and polypropylene oxide), such as further comprises a pharmaceutically acceptable amphiphilic block co-polymer comprising blocks of poly(ethylene oxide) and polypropylene oxide) in an amount of from 0.2% w/v to 20% w/v.
102. The composition according to any of embodiments 98 to 101, wherein the composition further comprises an adjuvant, such as an adjuvant selected from the group consisting of dexamethasone, B subunits of enterotoxin cholera toxin (CTB), TLR2 ligands, helminth-derived excretory/secretory (ES) products, rapamycin vitamin D3 analogues and aryl hydrocarbon receptor ligands.
103. The pharmaceutical composition according to any of embodiments 98 to 102, wherein the composition comprises said vector, e.g. said DNA plasmid, in a range of from 0.1 to 10 mg.
104. A method of treating a subject having an immune disease from the group consisting of autoimmune disease, allergic disease and graft rejection or being in need of prevention thereof, the method comprising administering to the subject a vector as defined in any of embodiments 1 to 94 or a pharmaceutical composition as defined in any of embodiments 98 to 103.
105. The method according to embodiment 104, wherein the vector or the pharmaceutical composition is administered in a therapeutically or prophylactically effective amount.
106. The method according to any of embodiments 104 to 105, wherein the vector or the pharmaceutical composition is administered by intradermal, intramuscular, or subcutaneous injection, or by mucosal or epithelial application, such as intranasal or oral.
107. A method of treating a subject having an autoimmune disease or being in need of prevention thereof, the method comprising administering to the subject a vector comprising a vector as defined in any of embodiments 1 to 41 or a pharmaceutical composition as defined in any of embodiments 98 to 103 comprising such vector.
108. The method according to embodiment 107, wherein the vector or the pharmaceutical composition is administered in a therapeutically effective amount.
109. The method according to any of embodiments 107 to 108, wherein the vector or the pharmaceutical composition is administered by intradermal, intramuscular, or subcutaneous injection, or by mucosal or epithelial application, such as intranasal or oral.
110. A method of treating a subject having an allergic disease or being in need of prevention thereof, the method comprising administering to the subject a vector comprising a vector as defined in any of embodiments 1 to 28 or 42 to 50 or a pharmaceutical composition as defined in any of embodiments 98 to 103 comprising such vector.
111. The method according to embodiment 110, wherein the vector or the pharmaceutical composition is administered in a therapeutically effective amount.
112. The method according to any of embodiments 110 to 111, wherein the vector or the pharmaceutical composition is administered by intradermal, intramuscular, or subcutaneous injection, or by mucosal or epithelial application, such as intranasal or oral.
113. A method of treating a subject having a graft rejection or being in need of prevention thereof, the method comprising administering to the subject a vector comprising a vector as defined in any of embodiments 1 to 28 or 51 to 56 or a pharmaceutical composition as defined in any of embodiments 98 to 103 comprising such vector.
114. The method according to embodiment 113, wherein the vector or the pharmaceutical composition is administered in a therapeutically effective amount.
115. The method according to any of embodiments 113 to 114, wherein the vector or the pharmaceutical composition is administered by intradermal, intramuscular, or subcutaneous injection, or by mucosal or epithelial application, such as intranasal or oral.

1. A vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit, wherein the antigenic unit comprises one or more T cell epitopes of a self-antigen, an allergen, an alloantigen or a xenoantigen; and
(b) one or more further nucleic acid sequences encoding one or more immunoinhibitory compounds,
wherein the vector allows for the co-expression of the first polypeptide and the one or more immunoinhibitory compounds as separate molecules.

2. The vector according to claim 1, wherein the one or more immunoinhibitory compounds induce immune tolerance and/or increase immune tolerance and/or maintain immune tolerance.

3. The vector according to any of claims 1 to 2, wherein the one or more immunoinhibitory compounds are an extracellular part, such as the extracellular domain, of an inhibitory checkpoint molecule.

4. The vector according to claim 3, wherein the inhibitory checkpoint molecule is selected from the group consisting of CLTA-4, PD-1, BTLA, LAG3, NOX2, SIGLEC7, SIGLEC9 and TIM-3, preferably a human inhibitory checkpoint molecule selected from the group consisting of hCLTA-4, hPD-1, hBTLA, hLAG3, hNOX2, hSIGLEC7, hSIGLEC9 and hTIM-3.

5. The vector according to any of claims 1 to 2, wherein the immunoinhibitory compound is a cytokine selected from the group consisting of IL-10, TGF-β1, TGF-β2, TGF-β3, IL-27, IL-2, GM-CSF, FLT3L, IFN-γ, IL-37 and IL-35, preferably a human cytokine selected from the group consisting of hIL-10, hTGF-β1, hTGF-β2, hTGF-β3, hIL-27, hIL-2, hGM-CSF, hFLT3L, hIFN-γ, hIL-37 and hIL-35.

6. The vector according to any of the previous claims, wherein the vector comprises multiple further nucleic acid sequences encoding more than one immunoinhibitory compound, such as 2, 3, 4, 5, 6, 7 or 8 immunoinhibitory compounds, such as 2, 3, 4, 5, 6, 7 or 8 different immunoinhibitory compounds.

7. The vector according to any of the previous claims, wherein said vector comprises one or more co-expression elements.

8. The vector according to claim 7, wherein said one or more co-expression elements cause the transcription of the first polypeptide and the one or more immunoinhibitory compounds on a single transcript and the independent translation into a separate first polypeptide and separate one or more immunoinhibitory compounds.

9. The vector according to any of claims 7 to 8, wherein the one or more co-expression elements are IRES elements or nucleic acid sequences encoding 2A self-cleaving peptides.

10. The vector according to claim 9, wherein said one or more co-expression elements cause the transcription of the first polypeptide and the one or more immunoinhibitory compounds as separate transcripts.

11. The vector according to claim 10, wherein said one or more co-expression elements are a) bidirectional promoters or are b) promoters, wherein the vector comprises a separate promoter for each of the nucleic acid sequences encoding the first polypeptide and the one or more immunoinhibitory compounds.

12. The vector according to any of the previous claims, wherein the antigenic unit comprises one or more T cell epitopes of a self-antigen.

13. The vector according to claim 12, wherein the antigenic unit comprises multiple T cell epitopes of one or more self-antigens.

14. The vector according to claim 13, wherein the one or more self-antigens are selected from the group consisting of self-antigens involved in multiple sclerosis, self-antigens involved in type 1 diabetes mellitus, self-antigens involved in celiac disease, self-antigens involved in rheumatoid arthritis, self-antigens involved in chronic inflammatory demyelinating polyradiculoneuropathy, self-antigens involved in Hashimoto's thyroiditis, self-antigens involved in Pemphigus foliaceus, self-antigens involved in Pemphigus vulgaris, self-antigens involved in thyroid eye disease, self-antigens involved in Grave's disease, self-antigens involved in primary biliary cirrhosis, self-antigens involved in myasthenia gravis, self-antigens involved in insulin-resistant diabetes, self-antigens involved in hemolytic anemia and self-antigens involved in psoriasis

15. The vector according to any of claims 1 to 12, wherein the antigenic unit comprises one or more T cell epitopes of an allergen.

16. The vector according to claim 15, wherein the antigenic unit comprises multiple T cell epitopes of one or more allergens.

17. The vector according to claim 16, wherein the one or more allergens are selected from the group consisting of shellfish allergen, cow's milk allergen, egg allergen, fish allergen, fruit allergen, wheat allergen, peanut allergen, tree nut allergen, soy allergen, seed allergen, buckwheat allergen, celery allergen, garlic allergen, gluten allergen, oat allergen, legumes allergen, maize allergen, milk allergen, mustard allergen, nuts allergen, poultry allergen, meat allergen, rice allergen, sesame allergen, bee venom allergen, vespid allergen, latex allergen, dust mite allergen, insect allergen, mold allergen, fungal allergen, furry animal allergen, pollen allergen and drug allergen.

18. The vector according to any of claims 15 to 17, wherein the antigenic unit comprises one or more T cell epitopes of a drug allergen of a drug selected from the group consisting of Factor VIII, insulin and therapeutic monoclonal antibody.

19. The vector according to any of claims 1 to 12, wherein the antigenic unit comprises one or more T cell epitopes of an alloantigen or xenoantigen.

20. The vector according to claim 12, wherein the antigenic unit comprises multiple T cell epitopes of one or more alloantigens or multiple T cell epitopes of one or more xenoantigens.

21. The vector according to any of the previous claims, wherein the antigenic unit comprises multiple discrete T cell epitopes which are separated by T cell epitope linkers.

22. The vector according to any of the previous claims, wherein the antigenic unit comprises multiple T cell epitopes which are minimal T cell epitopes comprised in one or more hotspots.

23. The vector according to any of the previous claims, wherein the targeting unit is or comprises a moiety that interacts with surface molecules on the antigen-presenting cell without activating the cell.

24. The vector according to any of the previous claims, wherein the targeting unit is or comprises a moiety that interacts with surface molecules on the antigen-presenting cell without inducing maturation of the cell.

25. The vector according to any of claims 23 to 24, wherein the surface molecule is selected from the group consisting of TGFβ receptor, such as TGFβR1, TGFβR2, and TGFβR3, IL-10R, such as IL-10RA and IL-10RB, IL-2R, IL-4R, IL-6R, IL-11R, IL-13R, IL-27R, IL-35R, IL-37R, GM-CSFR, FLT3, CCR7, CD11b, CD11c, CD103, CD14, CD36, CD205, CD109, VISTA, MARCO, MHCII, CD83, SIGLEC, Clec10A (MGL), ASGR (ASGR1/ASGR2), CD80, CD86, Clec9A, Clec12A, Clec12B, DCIR2, Langerin, MR, DC-Sign, Treml4, Dectin-1, PDL1, PDL2, HVEM, CD163 and CD141.

26. The vector according to claim 25, wherein the surface molecule is a surface molecule present on human antigen-presenting cells and wherein said surface molecule is selected from the group consisting of hTGFβ receptor, such as hTGFβR1, hTGFβR2, and hTGFβR3, hIL-10R, such as hIL-10RA and hIL-10RB, hIL-2R, hIL-4R, hIL-6R, hIL-11R, hIL-13R, hIL-27R, hIL-35R, hIL-37R, hGM-CSFR, hFLT3, hCCR7, hCD11b, hCD11c, hCD103, hCD14, hCD36, hCD205, hCD109, hVISTA, hMARCO, hMHCII, hCD83, hSIGLEC, hClec10A (hMGL), hASGR (hASGR1/hASGR2), hCD80, hCD86, hClec9A, hClec12A, hClec12B, hDCIR2, hLangerin, hMR, hDC-Sign, hTreml4, hDectin-1, hPDL1, hPDL2, hHVEM, hCD163 and hCD141.

27. The vector according to any of claims 23 to 26, wherein the moiety is a natural ligand, an antibody or part thereof, such as a scFv, or a synthetic ligand.

28. The vector according to claim 27, wherein the moiety is a natural ligand selected from the group consisting of TGFβ, such as TGF-β1, TGF-β2 and TGF-β3, IL-10, IL-2, IL-4, IL-6, IL-11, IL-13, IL-27, IL-35, IL-37, GM-CSF, FLT3L, CCL19, CCL21, ICAM-1, keratin, VSIG-3, preferably the extracellular domain of VSIG-3, SCGB3A2, CTLA-4, preferably the extracellular domain of CTLA-4, PD-1, preferably the extracellular domain of PD-1 and BTLA, preferably the extracellular domain of BTLA.

29. The vector according to claim 28, wherein the moiety is a human natural ligand selected from the group consisting of hTGFβ, hIL-10, hIL-2, hIL-4, hIL-6, hIL-11, hIL-13, hIL-27, hIL-35, hIL-37, hGM-CSF, hFLT3L, hCCL19, hCCL21, hICAM-1, hkeratin, hVSIG-3, preferably the extracellular domain of hVSIG-3, hSCGB3A2, hCTLA-4, preferably the extracellular domain of hCTLA-4, hPD-1, preferably the extracellular domain of PD-1 and hBTLA, preferably the extracellular domain of hBTLA.

30. The vector according to any of claims 23 to 29, wherein the targeting unit is or comprises hIL1-10, hTGFβ, such as hTGFβ-1, hTGFβ-2 and hTGFβ-3, hSCGB3A2 or hVSIG-3, preferably the extracellular domain of hVSIG-3.

31. The vector according to any of the previous claims, wherein the multimerization unit is selected from the group consisting of dimerization unit, trimerization unit, such as a collagen-derived trimerization unit, such as a human collagen-derived trimerization domain, such as human collagen derived XVIII trimerization domain or human collagen XV trimerization domain or the C-terminal domain of T4 fibritin and tetramerization unit, such as a domain derived from p53 and wherein said multimerization unit optionally comprises a hinge region, such as hinge exon h1 and hinge exon h4.

32. The vector according to claim 31, wherein the vector comprises a hinge region which has the ability to form one or more covalent bonds and is preferably Ig derived.

33. The vector according to any of claims 31 to 32, wherein the multimerization unit is a dimerization unit and said dimerization unit further comprises another domain that facilitates dimerization, preferably wherein the other domain is an immunoglobulin domain, more preferably an immunoglobulin constant domain.

34. The vector according to claim 33, wherein the other domain is a carboxyterminal C domain derived from IgG, preferably from IgG3.

35. The vector according to any of claims 33 to 34, wherein the dimerization unit further comprises a dimerization unit linker, such as glycine-serine rich linker, such as GGGSSGGGSG and preferably wherein the dimerization unit linker connects the hinge region and the other domain that facilitates dimerization.

36. The vector according to any of claims 33 to 35, wherein the dimerization unit comprises hinge exon h1 and hinge exon h4, a dimerization unit linker and a CH3 domain of human IgG3.

37. The vector according to any of the previous claims, wherein the first nucleic acid sequence encodes a first polypeptide which further comprises a unit liker that connects the antigenic unit to the multimerization unit, and wherein the unit linker is a non-immunogenic linker and/or flexible or rigid linker.

38. The vector according to any of the previous claims, wherein the first nucleic acid sequence encodes a first polypeptide which further comprises a signal peptide and preferably wherein also the one or more further nucleic acid sequences further encode a signal peptide.

39. The vector according to any of the previous claims, wherein the vector is a viral vector, such as an RNA viral vector or DNA viral vector or a plasmid, such as an RNA plasmid or DNA plasmid.

40. A method of producing a vector as defined in any of the previous claims, the method comprising:
a) transfecting cells in vitro with the vector;
b) culturing said cells;
c) optionally, lysing the cells to release the vector from the cells; and
d) collecting and optionally purifying the vector.

41. A host cell comprising a vector as defined in any of claims 1 to 39, such as a host cell selected from the group consisting of prokaryote cells, yeast cells, insect cells, higher eukaryotic cells such as cells from animals or humans.

42. A vector as defined in any of claims 1 to 39 for use as a medicament.

43. A pharmaceutical composition comprising the vector as defined in any of claims 1 to 39 and a pharmaceutically acceptable carrier or diluent.

44. The pharmaceutical composition according to claim 43, wherein the composition further comprises a transfection agent.

45. The pharmaceutical composition according to any of claims 43 to 44, wherein the composition further comprises an adjuvant, such as an adjuvant selected from the group consisting of dexamethasone, B subunits of enterotoxin cholera toxin (CTB), TLR2 ligands, helminth-derived excretory/secretory (ES) products, rapamycin vitamin D3 analogues and aryl hydrocarbon receptor ligands.

46. The pharmaceutical composition according to any of claims 43 to 45, wherein the composition comprises said vector, e.g. said DNA plasmid, in a range of from 0.1 to 10 mg.

47. A method of treating a subject having an immune disease from the group consisting of autoimmune disease, allergic disease and graft rejection or being in need of prevention thereof, the method comprising administering to the subject a vector as defined in any of claims 1 to 39 or a pharmaceutical composition as defined in any of claims 43 to 46.

48. A method of treating a subject having an autoimmune disease or being in need of prevention thereof, the method comprising administering to the subject a vector as defined in any of claims 1 to 14 and 21 to 39 or a pharmaceutical composition as defined in any of claims 43 to 46 comprising such vector.

49. A method for treating a subject having an allergic disease or being in need of prevention thereof, the method comprising administering to the subject a vector as defined in any of claims 1 to 11, 15 to 18 and 21 to 39 or a pharmaceutical composition as defined in any of claims 43 to 46 comprising such vector.

50. A method for treating a subject having a graft rejection or being in need of prevention thereof, the method comprising administering to the subject a vector as defined in any of claims 1 to 11 and 19 to 39 or a pharmaceutical composition as defined in any of claims 43 to 46, comprising such vector.

51. The method according to any of claims 47 to 50, wherein the vector or the pharmaceutical composition is administered in a therapeutically or prophylactically effective amount, such as administered by intradermal, intramuscular, or subcutaneous injection, or by mucosal or epithelial application, such as intranasal or oral.
